(19) United States
(12) Patent Application Publication
(10) Pub. No.: US 2025/0114287 A1
(43) Pub. Date: Apr. 10, 2025

(54) COLOR COATINGS WITH DOWNSTREAM FEATURES

(71) Applicant: HFC Prestige Service Germany GmbH, Darmstadt (DE)

(72) Inventors: Mathias Kurt Herrlein, Kronberg (DE); Graham Neil Mckelvey, Hesse (DE); Matija Crne, Wiesbaden (DE); Simon Paul Godfrey, Oberursel (DE); Corinne Violette Mohr, Lorsch (DE); Ingo Weber, Basel (DE); Swapna Pinakattu, Reinheim (DE); Patrick Alexander Kiefer, Darmstadt (DE); Petra Barbara Braun, Münster (DE); Andrej Gross, Darmstadt (DE); Felix Herkner, Eppstein (DE); Axel Meyer, Frankfurt am Main (DE); Carl Uwe Oswald Ludwig Schmidt, Reinheim (DE); Michael A. Brook, Ancaster (CA); Timothy Robert Clark, Obermichelbach (DE); Jüergen Karl Anton Schatz, Adelsdorf (DE); Claus Schreiner, Osterholz-Scharmbeck (DE)

(21) Appl. No.: 18/925,903

(22) Filed: Oct. 24, 2024

Related U.S. Application Data
(63) Continuation of application No. 17/362,397, filed on Jun. 29, 2021.
(60) Provisional application No. 63/046,110, filed on Jun. 30, 2020.

(51) Int. Cl. A61Q 5/10; A61K 8/893; A61K 8/898

(52) U.S. Cl. CPC A61K 8/585; A45D 19/005; A45D 19/0066; A61K 8/89; A61Q 5/06; A61Q 5/065; A61Q 5/10; A61Q 5/12; A61K 2800/43; A61K 2800/4324; A61K 2800/884; A61K 2800/95

(57) ABSTRACT

Aspects of the invention are directed to characteristics and parameters of color coatings on keratin fibers. The color coatings are produced from compositions of several kinds of polymers that enable the color coatings to exhibit adherence, remanence, wear fastness and resistance to environmental attack.

### BACKGROUND

The recent decade has seen significant effort directed toward development of a hair coloring technology that can be characterized as permanent but avoids bleach and penetration of dye precursors into the keratin fiber cortex. This technology in the main involves coating the surfaces of keratin fibers (e.g., scalp hair) with a polymer film containing pigment particles. Numerous difficulties have plagued this development, however. Water soluble polymer films are easily removed by shampoo. Water insoluble films seem to have better remanence but they also typically survive only two or three shampoo washings. Film inflexibility, weakness and thickness lead to flaking, fiber breakage. Film application through use of dispersions or solutions of components in liquid media often lead to incomplete distribution of pigment particles and noticeably artificial coloration due to the different qualities of the roots, mid-lengths and tips of keratin fibers. Methods for treatment of the keratin fibers with the dispersions or solutions are problematic because of dripping, staining, coat flow, clumping and lumping of gels, component separation and difficulties with color matching.

The kinds of polymers range from preformed organic polymers and silicone polymers optionally containing functional groups to reactive-polymer compounds that react in situ to form polymer chains and cross links. Mixtures and layers of polymers are typical in this respect. Suitability and compatibility of these polymers with each other and with hair and scalp are problematic. Failure to establish compatible inter-surface connections can lead to flaking and easy shampoo removal. Strong inter-surface connections on the other hand can lead to extreme difficulty in attempts to remove and/or replace the colored film with another colored film. Rapid setting and lack of flowability of the compositions for application lead to patchy coverage instead of contiguous film formation. Still other difficulties involve regulatory requirements and the need to avoid tissue damaging and/or environmental attack by sunlight, UV, wind, rain and airborne chemicals, degrading chemicals in water and in hair care and hair dye compositions, as well as sweat and sebum.

### SUMMARY OF THE INVENTION

These and other difficulties are addressed by aspects of the present invention. The present invention meets these objectives through the design of the compositions for application, design of the coating on keratin fibers and/or keratinous surfaces such as but not limited to human hair adjustment of starting materials and control of application methods. These multiple design features enable easy application of the compositions to be dressed onto keratin fibers. These multiple design features enable achievement of contiguous film formation under processing parameters that enable unhurried dressing but rapid film formation when desired. These multiple design features enable robust remanence, long wear-fastness, uniform color distribution and/or varied color distribution as well as establishing triggers for color coating removal. These multiple design features enable avoidance of “downstream problems” that can otherwise be associated with the method for treatment, care of the colored hair and maintenance of the colored hair as it is assailed by environmental factors including but not limited to UV rays, sebum, shampoo, brushing, rinsing, rain, wind, coverings by scarves and hats, rubbing and drying with towels and hair dryers, hair conditioners, styling hair sprays, hot iron curling and other environmental and hair care factors. At the same time these multiple design features according to the invention provide tactile, visual, sound and olfactory sensations at least similar to untreated hair. According to the invention, the design features provide colored hair having tactile properties such as light, fluffy, free flowing, non-sticky and non-matted and other physical interactions of multiple colored hair strands that are similar to the interactions of natural, non-colored hair of humans.

These and other design features of aspects of the present invention include but not limited to color coatings on keratin fibers/keratinous surfaces, color compositions and their components, parameters associated with treatment of keratin fibers/keratinous surfaces with the color compositions and parameters associated with formation of color coatings on keratin fibers/keratinous surfaces produced by treatment with the color compositions. These aspects additionally include qualities of the color coatings that deliver the above-described characteristics for colored keratin fibers/keratinous surfaces.

Embodiments of the color coatings are directed to polymeric films with pigment particles on keratin fibers/keratinous surfaces (hereinafter keratin fibers), preferably human scalp hair. The polymeric films may comprise one, two, three or four polymeric layers overlaid on the keratin fibers. The color coatings, also known as color polymeric films on keratin fibers, display a set of physical and chemical properties that characterize the coatings irrespective of the kind or kinds of polymers and pigment particles forming the color coatings. These characteristics may be established by the color coating properties including but not limited to remanence, wash-fastness, resistance to environmental attack, elastomeric flexibility to enable free movement of the coated keratin fibers, tensile strength to resist flaking and breakage, and color mimicking of appropriate shades for roots, mid-length and tips of keratin fibers. These characteristics preferably may be demonstrated by tests of the color coatings in hair swatches prepared from natural, unbleached natural white human hair and bleached natural white human hair.

Embodiments of the color compositions of present invention are also directed to a group of preformed polymer and reactive-polymer compositions containing pigment particles that may be dressed onto the surfaces of keratin fibers (e.g., human scalp hair) to form colored coatings on these surfaces. The techniques for dressing are accomplished at rates and timing that enable ready segmental coating of roots, mid-lengths and tips of keratin fibers and avoid and/or minimize dripping, clumping, lumping, balling and/or avoid flow and spread issues of the process for applying the compositions to the fiber surfaces. Embodiments of the present invention are further directed to pre-treatment compositions, optional topcoat compositions and optional post care compositions for use with the color compositions according to the invention. The pre-treatment compositions may be dressed onto the keratin fiber surfaces before dressing with the color compositions. The optional topcoat compositions may be dressed onto the keratin fibers coated with film layers formed from the pre-treatment compositions and color compositions. The optional topcoat compositions may be dressed onto the coated keratin fibers to provide additional brilliance, shine, resistance to environmental attack and hair arrangement control. The post-treatment care composition provide smoothing and free flowing qualities to the keratin fibers as well as acting as sacrificial material to ward away environmental attack. The pre-treatment compositions as well as the color compositions may contain mixtures and blends of pigment microparticles with dispersant and optional components for facilitating pigment microparticle combination with one or more of the pre-treatment and color compositions. The pre-treatment compositions, color compositions and topcoat compositions also may optionally include any one or more of a number of additives herein described as a group characterized as the additive composition. The additive composition is not a separate composition for addition to keratin fibers. Instead, one or more of the constituents of the additive composition may be combined with any one or more of the pretreatment composition, the color composition and/or the topcoat composition. The additives provide certain beneficial qualities to any one or more of these compositions. Such qualities include but are not limited to UV and sunlight resistance, resistance to triplet formation, sebum resistance, hair style setting and the like. According to the invention, the dressing of the keratin fibers with the pre-treatment and color compositions as well as with the optional topcoat compositions produces multiple layer color coatings on keratin fibers that display significant remanence, maximum color wash-fastness, shine, shine retentions, smoothness, softness, silkiness, hair feel, resistance to removal by sebum, UV, rubbing, shampoo, hair dryer blowing, wind, flaking, cracking while at the same time enabling trigger techniques for removal not associated with any of these assailing factors.

Embodiments of color compositions according to the invention may comprise preformed color compositions of one or more preformed organic, silicone and/or organosilicone polymers, oligomers and/or small molecules. The small molecules may have single molecule weights while the oligomers and polymers may range from low to mid-range to high-range weight average molecular weights (hereinafter Mw or Mw's) of at least about 100 Da up to MDa ranges. The small molecules, oligomers and polymers may include hydrophilic and lipophilic non-reactive functional groups. The preformed color compositions also comprise pigment particles combined with dispersant and an organic or organic-aqueous medium. The preformed compositions contain groups that enable hydrogen bonding and dipolar interactions. The preformed compositions may also contain lipophilic groups and/or chains that establish entanglement and/or van der Waals interactions. The combination of hydrogen bonding and entanglement parameters provides design parameters for the preformed color compositions.

Embodiments of the preformed color compositions may comprise more than one individual preformed composition. Typically, multiple preformed color compositions are sequentially applied to provide colored coatings of multiple layers. At least one layer may contain pigment particles and pigment particles may be present in more than one and/or in all layers. The individual preformed color compositions forming the layers exhibit mutual compatibility and the layer adjacent to the keratin fiber surfaces preferably will exhibit compatibility with the molecular constituents of the keratin surfaces.

Embodiments of color compositions according to the invention may comprise reactive-polymer color compositions. In addition to dispersed pigment particles, the reactive-polymer color compositions may comprise at least two reactive-polymer components having complementary reactive groups that covalently combine in situ to form longer polymer chains and cross-linked individual chains. The reactive-polymer components may comprise organic reactive-polymers, silicone reactive-polymers, organosilicone reactive-polymers and/or condensable silanes of 1 to 20 or more silicons bearing hydroxyl and/or alkoxy groups. The reactive-polymers may be polymers, oligomers and/or small molecules. As small molecules, the reactive polymers may be unitary, dimer, trimer, tetramer and similar units such as monomeric units that combine to form extended oligomeric and/or polymeric chains. The reactive polymers may have small molecule and unitary monomeric weights and/or as multiple monomeric units and/or oligomer and polymer low range Mw's such as from about 100 Da and when combined in situ to form longer chain cross linked polymers, the in situ formed polymers may have Mw's of at least 5 KDa up to a high MDa range for the extended chains to an almost infinite Mw for network cross linked in situ formed polymers. The cross linked in situ formed polymers may have at least one internal chain and/or one chain terminal cross link per at least 100 monomeric residues, preferably at least 50 monomeric residues, more preferably at least about 10 monomeric residues, most preferably at least about 1 to 3 monomeric residues. Exemplary cross links may extend the reactive polymers/oligomers/small molecule reactants multiple times to provide polymeric chains of at least 5 KDa and may internally cross link such extended polymeric chains by at least one and preferably at least 2, 3 or 4 internal links so as to form network and/or star configurations of the crosslinked polymeric product.

The complementary reactive groups are capable of forming covalent bonds to join reactive-polymer molecules to extend their chains and to cross-link to form nets, stars, branches and all forms of inter-linked molecules. The complementary reactive groups are suitable for covalent bonding in a biological environment. In particular, these groups combine without injuring adjacent tissue or causing damage to keratin fibers and are regarded as environmentally and medically acceptable by regulatory authorities. The heat, rate of reaction, side effects and reaction by-products are controlled so as to be amenable with biological tissues. Embodiments of the complementary reactive groups include but are not limited to: a) hydroxysilyl and alkoxysilyl; b) amine and/or mercaptan and α, β unsaturated ketoxy, acyl and acyloxy groups such as but not limited to (meth)acryloxy and crotonyloxy; c) carbodiimide and carboxyl or amine; d) amine or hydroxyl and isocyanate or a non-isocyanate urethane-forming-material such as a poly cyclic carbonate; e) hydridosilyl and vinyl silyl; and, f) activated reactive groups for amide and ester formation.

Embodiments of the pre-treatment, and color compositions (including the preformed and the reactive-polymer color compositions) are formulated in media, with optional additional components, and at concentrations that enable facile dressing onto the keratin fiber surfaces and effective film formation by drying and/or curing. The pre-treatment and color compositions are formulated to be liquids with appropriate surface free energy, surface tension and contact angle relative to the surfaces to which they are dressed to enable complete spreading but with viscosities to minimize or avoid run-off and/or dripping. The drying and curing rates of the pre-treatment and color compositions following their dressing onto keratin surfaces are appropriate for promotion of efficient unhurried treatment and applications. The dried and cured films formed from the pre-treatment and color compositions display at least some of the following properties elastomeric, entanglement, Mw's, hydrogen bonding, polar/dipolar and lipophilic interactions and cross-linking densities that promote adhesion, remanence, resistance toward sebum, wind, brushing, rubbing and unacceptable degradation. The optional topcoat and post-treatment formulations add to the promotion of shine, silkiness, smoothness, fluffiness, and non-sticky character associated with the dried and cured pre-treatment and color compositions formed as coating layers on the keratin fibers and may further drive the color remanence.

Certain embodiments of the pre-treatment and color compositions of the invention may be arranged to provide custom color combinations based upon the desired color and character and condition of the keratin fibers (hair) to be dressed. The pre-treatment and color compositions are pre-formulated without pigment particles in accord this aspect. Alternative embodiments provide a plethora of pre-treatment and color compositions with dispersed pigment microparticles balanced to enable selections of complete preformulated compositions for providing colored coatings of keratin fibers. The custom embodiments for forming colored coatings on keratin fibers are enabled by spectroscopic and microscopic examination of the keratin fibers and by keratin fiber area location on the scalp as well as by the historical information of the keratin fibers to be dressed. According to this embodiment, the information is inputted into a computer loaded with one or more software programs to analyze the information, input the desired colorations for roots, mid lengths and tips of keratin fibers located at several regions of the scalp and determine the combinations of microparticle pigments to combine to achieve the desired results. The microparticle combinations are preferably made according to a complex mathematical formula known as the Sendyureva-Godfrey expressions. The total resulting color expression is given by the follow equation

$${R}_{Total} ( λ ) = {R}_{f i l m} ( λ ) + \frac{{T}_{f i l m}^{2} ( λ ) {R}_{b a s e} ( λ )}{1 - {R}_{f i l m} ( λ ) {R}_{b a s e} ( λ )}$$

Where R_{Total} represents the total amount of light reflected from the colored film on the keratin fibers, R_{film} represents the light reflected from the surface of the colored film, T_{film} represent the amount of light that is transmitted through the film, and R_{base} represents the light represented from the base below the film, in this case the keratin fiber surface. The functions are all wavelength dependent as shown by the term (λ). Those skilled in the art will understand that the function R_{Total}(λ) can be used to calculated the observed color under different light sources, for example to give the color in terms of the L*a*b (CIELAB, Commission Internationale de l'éclairage) coordinates. While R_{base}(λ) can be experimentally determined using methods well known by those skilled in the art, the terms R_{film} and T_{film} can be modelled themselves using the follow equations.

$${R}_{f i l m} ( λ ) = \frac{1}{( \frac{{S}_{T O T} + {K}_{T O T}}{{S}_{T O T}} ) + {( {( \frac{{S}_{T O T} + {K}_{T O T}}{{S}_{T O T}} )}^{2} - 1 )}^{1 / 2} ( coth ( {{DS}_{T O T} ( {( \frac{{S}_{T O T} + {K}_{T O T}}{{S}_{T O T}} )}^{2} - 1 )}^{1 / 2} ) )}$$

$${T}_{f i l m} ( λ ) = \frac{1}{( \frac{{S}_{T O T} + {K}_{T O T}}{{S}_{T O T}} ) ( sinh ( {{DS}_{T O T} ( {( \frac{{S}_{T O T} + {K}_{T O T}}{{S}_{T O T}} )}^{2} - 1 )}^{1 / 2} ) ) + ( \frac{{S}_{T O T} + {K}_{T O T}}{{S}_{T O T}} ) ( cosh ( {{DS}_{T O T} ( {( \frac{{S}_{T O T} + {K}_{T O T}}{{S}_{T O T}} )}^{2} - 1 )}^{1 / 2} ) )}$$

wherein S_{TOT} and K_{TOT} are the total S and K terms for the film, the scattering and absorbing components and D is the depth of the film on the keratinous surface.
With the following terms derived from

$$\begin{matrix}{S}_{T O T} = ( 1 - {\sum }_{i = 0}^{i = n} {F}_{P i} ) {S}_{f i l m} + {\sum }_{i = 0}^{i = n} {F}_{P i} {S}_{P i} \\ {K}_{T O T} = ( 1 - {\sum }_{i = 0}^{i = n} {F}_{P i} ) {K}_{f i l m} + {\sum }_{i = 0}^{i = n} {F}_{P i} {K}_{P i}\end{matrix}$$

Where S_{film} and K_{film} are the scattering and absorbing properties of the film itself without pigments and the term i refers to the individual pigments used within the film, and S_{Pi} and K_{Pi} refer to the individual pigments scattering and absorbing terms, and F_{Pi} refers to the fraction of each pigment within the film. Net when no pigments are added the terms collapse to give just the films scattering and absorbing properties. The individual terms for the film and pigments can be experimentally determined by those skilled in the art. This series of equations are used to create the Sendyureva-Godfrey expression for predicting the color of a given film. Using regular optimization routines, the levels of pigment to add can be calculated to give a desired color result with a given thickness of film on the hair. These expression are further modified when metal flakes are used due their properties not being described as regular scattering particles.

This formula takes into account the individual absorbing and scattering properties of each type of microparticle, together with the material around the microparticles to predict the optical properties of said coating. This can be used to optimize the combination of microparticles and their levels to give the desired color result, and also to understand the available colors for a give combination of starting microparticles. The computer output will provide combinations of components involving mixtures of differing hues and shades of microparticles as well as dispersant variations and concentrations of microparticles in the colored compositions. The computer software output may also be electronically connected with an automated mixing system with vessels separately containing the pre-treatment composition, the color composition minus microparticles and a plethora of different hues and shades of microparticles in a microparticle composition also containing at least dispersant. The automated mixing system is designed to carry out mixing instructions from the computer software output and provide custom portions of pre-treatment compositions and color compositions for dressing separately individual areas of keratin fibers on the scalp and separately dressing roots, mid lengths and tips of the fibers of such sections.

Embodiments of the pre-treatment compositions, color compositions and optional topcoat compositions preferably are constructed with viscosity, densities, component concentrations and solvent identities to enable appropriate contact angles of approximately zero degrees and strong inter-surface adhesion/adherance. These parameters enable the process of dressing these compositions onto keratin fibers and keratin fibers to achieve essentially complete spreading along the desired surfaces or portions of surfaces of keratin fibers. These parameters also minimize and/or substantially avoid dripping, scattering and flowing off of the keratin fibers, of the composition being applied.

Embodiments of the pre-treatment compositions and color compositions preferably are formulated with preformed polymers and/or reactive-polymers that will display a combination of: a) adherence to underlying and overlying films and/or substances such as keratin fiber surfaces; and b) a balance of elastomeric, entanglement and film integrity properties enabling the formed film to stretch, elongate and recover without losing adherence to underlying and overlying films and/or substances such as keratin fiber surfaces while maintaining continuous film integrity so as to avoid flaking, breaking and/or shedding. The adherence, elastomeric and entanglement properties of the films formed from the color compositions are functions of the Mw, molecular weight distribution, hydrogen, dipolar, lipophilic and van der Waals interactions, cross linking density, degree of polymer branching and density of non-reactive functional groups per polymer molecule or number of molecules including but not limited to urethane, urea, amide, ester, amine, mercaptan (thiol), hydroxyl and in some embodiments, carboxylic acid. The adherence, elastomer and entanglement properties of the films are as well functions of the chemical make-up of the polymers themselves including but not limited to: a) organic chain polymers exemplified by carbon-carbon chains formed from olefins, carbon-carbon chain segments connected by polymeric forming groups including but not limited to ester, urethane, urea, amide, ether oxygen exemplified by polyethylene glycol segments; b) silicone chain polymers exemplified by polysilicones, MDTQ silicone segments both of which may contain organic side chains of the foregoing exemplified descriptions; and, c) organosilicones exemplified by polysilicone and MDTQ silicone segments combined with organic chains as pendant and in chain segments. The organic, silicone and organosilicone polymers may exhibit random distribution of monomeric residues within the molecular chains, block distribution of monomeric residues and/or differing monomer segments within the molecular chains, star distributions of differing monomer segments, as well as other known monomeric and/or monomeric segment distributions typically and commonly present in organic, silicone and organosilicon polymers.

Embodiments of the pre-treatment and/or color compositions may include any one or more of the constituents of the additive composition including but not limited to plasticizers. dispersants, synthetic and/or natural clays, synthetic and/or natural microfibers and function agents designed to contribute to the elasticity, remanence, maximum color wash-fastness, shine, shine retentions, smoothness, softness, silkiness, hair feel as well as to contribute to resistance to removal and or coating damage by sebum, UV, rubbing, shampoo, hair dryer blowing, wind, flaking, cracking of the film coatings on keratin fibers. Plasticizers for organic, silicone and organosilicon polymers may be added to the pre-treatment and/or color compositions to increase elasticity, film integrity and interlayer compatibility. Dispersants including emulsifiers, surfactants and suspension agents may be added to the pre-treatment and/or color compositions to positively affect uniform, substantially permanent dispersions of dis-similar substances and solid particles therein. Clays and microfibers may be added to the pretreatment and/or color compositions to strengthen film integrity add smoothness to the tactile sensation of the dried/cured film coatings and enable elongation and stretching without film breakage or flaking.

Cellulosic derivatives and/or substances that are incompatible with the polymers of the pre-treatment and color compositions may be added in small concentrations to form domains of the cellulose derivatives and/or incompatible substances within the polymer films. These domains display complete insolubility and resistance to water rinsing and shampoo treatments but are highly soluble in certain organic solvents such as ethanol or isopropanol so that use of a shampoo treatment also containing a reasonable and topically acceptably concentration of such organic solvents will dissolve the domains and enable breakup and removal of the polymer films. Wax micro-particulates having melt temperatures at least 25° C. higher than body temperature may also be combined with the pre-treatment and/or color compositions and/or topcoat compositions and will be kept in substantially uniform dispersions when emulsifiers and surfactants are also included. Use of a hot air hair dryer will enable melting of the wax micro-particulates and will enable breakup and removal of the polymer films.

Embodiments of the pre-treatment and color compositions may be formulated to provide polymer concentrations in media, identity of media and degree and density of reactive-polymer complementary reactive groups to enable efficient, un-hurried dressing of the compositions onto areas and sections of keratin fibers. The polymer concentrations, identity of media, degree and density of complementary reactive groups are low enough to maintain a slow dry/cure rate for forming the films yet high enough to avoid and/or minimize dripping, sloughing and coverage of erroneous sections and/or areas of scalp keratin fibers and to prevent the need of waiting an unacceptable period of time before the hair can be washed. The concentrations, identities, degree and density for the pre-treatment and color compositions also enable removal of the compositions before drying and/or curing in the event that mistakes are made or changes are to be instituted.

Embodiments of the optional topcoat may be dressed as a coating on the keratin fibers already coated with films formed from the pre-treatment and color compositions according to the foregoing aspects of the invention. The topcoat composition may comprise a colorless combination of polymeric olefinic ester in aqueous organic solvent similar to hair styling sprays as well as any one or more of the constituents of the additives composition described above. In addition to the usual ingredients of the topcoat composition, the topcoat composition may also comprise one of the first and second components of the reactive polymer composition of the color composition. In this alternative, the first or second component present in the topcoat composition will have a reactive functional group that is complementary to one of reactive complementary pairs of the reactive polymer composition. The first or second component in the topcoat composition will combine with its complementary reactive functional group of the reactive polymer composition already dressed onto the keratin fibers. Preferably, the first or second component of the reactive polymer composition that is complementary to the first or second component of the topcoat will be in at least slight excess in the reactive polymer composition. The excess concentration will enable covalent bonding of the topcoat to the color coat through this complementary reactive pair. The covalent bonding will provide covalent bond adherence between the topcoat and the colored coat dried and cured on the keratin fibers. Unless the topcoat composition contains a concentration of first or second component of the reactive polymer composition, the topcoat composition may be applied to the integrated, dried/cured film of colored coatings on the keratin fibers. If the topcoat composition also contains a concentration of a first or second component, the topcoat may be applied over the color composition already present on the keratin fibers but not yet fully dried and cured. Hot air drying of the topcoat composition, and curing when appropriate, provides a hair setting, environmentally resistant styling option of a highly water, shampoo, sebum, UV, sunlight and wind resistant film overcoating the colored coatings on the keratin fibers. The topcoat film may lessen the fluffiness and looseness of the keratin fibers and can function to maintain a certain hair styling during exposure to wind, rain and similar environmental factors. The topcoat may be removed by vigorous brushing with a stiff bristle brush.

Embodiments of the care treatment compositions may comprise lubricants and sacrificial liquids of sufficient density to remain as a post-treatment coating on keratin fibers already coated with films produced from the pre-treatment and color compositions. The lubricants enhance shine, fluffiness, tactile free-flowing character of so coated keratin fibers.

Preferred embodiments of the pre-treatment compositions comprise an “aza” polymer (multivalence nitrogen in monomeric chain unit of organic polymer) with amine side chains such as but not limited to linear and/or branched polyethyleneimine, and polysilicone with amine side chains preferably bound to alternating silyl groups of the polysilicone chain. The Mw of the pre-treatment composition polymers may range from 1 KDa to 1 MDa or higher. Preferred color compositions directed to the preformed polymer compositions comprise preformed polymers having in-chain coupling groups including urethane, urea, amide and/or ester. The preferred preformed polymers may also comprise similar pendant groups. The preformed polymers may comprise any chain configuration including but not limited to linear, branched, network, star and any combination thereof. The Mw of the preferred preformed polymers may range from 5 KDa to 1 MDa. The functional group equivalent weight (FGEW) of in-chain coupling groups of the preferred preformed polymers may range from 250 Da to 5 KDa. The equivalent Mw for hydrogen bonding and dipolar interactions of the preferred preformed polymers may range from 250 Da to 10 KDa. The Mw for entanglement of the preferred preformed polymer preferably resulting from linear and branched chain lipophilic interaction may range from 4 KDa to 50 KDa. The combination of equivalent Mw's for hydrogen bonding and dipolar interactions of the preferred pre-formed polymer may range from 250 Da to 50 KDa.

Preferred color compositions comprise reactive-polymer color compositions of organic and/or silicone chains having complementary reactive groups such as but not limited to the complementary groups of amine and a p unsaturated acyl groups, the complementary reactive groups of carboxylic acid and carbodiimide, the complementary reactive groups of hydroxyl/amine and isocyanate derivatives, and the complementary reactive groups of methoxy and/or ethoxy silyl groups. The reactive-polymer molecules may comprise olefinic oligomers substituted only by the complementary reactive groups or may comprise urethane, urea, ester and/or amide oligomers substituted by complementary reactive groups. The Mw of the reactive-polymers range from 120 Da to 500 KDa with complementary reactive group densities ranging from at least 2 per molecule, preferably at least 3 per molecule and more preferably at least 1 per one, two, three or four monomeric units. The Mw of the polymer products resulting from reaction of the reactive-polymers may be at least 5 KDa to almost infinite Mw with cross link densities providing at least one cross link per 5 molecules and preferably at least two to five cross links per each molecule. The equivalent distribution Mw of in-chain functional groups of the polymer products resulting from the reaction coupling of may range from 120 Da to 50 KDa. The equivalent Mw for hydrogen bonding and dipolar interactions of the polymer products resulting from reaction of the preferred reactive-polymers may range from at least 100 Da. The equivalent Mw for entanglement of linear and branched chain lipophilic interactions of the polymer products resulting from preferred reactive-polymers may range from at least 1 KDa. The combination of equivalent Mw's for hydrogen bonding, dipolar interactions and entanglements of the preferred polymer products resulting from preferred reactive-polymers may range from at least 100 Da.

One or more of the preferred pre-treatment, preferred preformed polymer and reactive-polymer compositions may comprise one or more pigment microparticles with dispersant. The concentration of pigment particles in the pre-treatment and/or preformed and/or reactive-polymer compositions may range from 0.1 wt percent to 10 wt percent relative to the total weight of the composition. The concentration of dispersant is sufficient to support a substantially uniform dispersion of the pigment in the designated composition for at least 4 hours and preferably at least 10 hours and indefinitely preferably with intermittent mild agitation such as by gently shaking.

Embodiments for dressing the preferred pre-treatment, preformed and reactive-polymer compositions and optional topcoat onto keratin fibers may be accomplished by spreading the pre-treatment composition on the keratin fibers to essentially to completely form a pre-treatment coating of the keratin fibers. The media of the pre-treatment composition is at least partially evaporated by drying with forced air and/or heated forced air. The preformed polymer composition and/or the reactive-polymer composition may be dressed separately on sections of keratin fibers and on areas of keratin fibers on the scalp. Applied preformed polymer composition may be set by removal of medium through application of forced air and/or heated forced air. Applied reactive-polymer composition may be dried and cured through application of forced air and/or heated forced air and/or as appropriate, irradiation with electromagnetic radiation of appropriate selected wavelength for the complementary reactive functional group pair present. The optional topcoat may be dressed onto the keratin fibers already dressed with pretreatment composition and color composition (preformed and reactive polymer compositions). As discussed above, when the topcoat composition comprises typical constituents, it may be dressed onto the fully dried and cured pretreatment and color compositions. When the topcoat composition also contains one of the first and second components of the reactive polymer composition, the topcoat composition may be dressed onto the keratin fibers having partially dried and cured pretreatment and color compositions.

### BRIEF DESCRIPTION OF FIGURES

FIG. 1 illustrates a color gamut plot.

FIG. 2 illustrates a color combination plot.

FIG. 3 illustrates a color combination plot.

FIG. 4 illustrates a color combination plot.

FIG. 5 illustrates a color combination plot.

FIG. 6 illustrates a color combination plot.

FIG. 7 illustrates the pigments according to Scheme 1.

### DEFINITIONS

Unless defined otherwise, all technical and scientific terms used herein have the same meaning as commonly understood by a person of ordinary skill in the art.

As used in the specification and the appended claims, the singular forms “a,” “an” and “the” include plural referents unless the context clearly dictates otherwise

The term “may” in the context of this application means “is permitted to” or “is able to” and is a synonym for the term “can.” The term “may” as used herein does not mean possibility or chance.

The term and/or in the context of this application means one or the other or both. For example, an aqueous solution of A and/or B means an aqueous solution of A alone, an aqueous solution of B alone and an aqueous solution of a combination of A and B.

The molecular weight of a polymer or oligomer used according to the invention may be measured by a weight average molecular weight, and the distribution of molecules of different molecular weights of a polymer or oligomer used according to the invention is determined by its polydispersity index. Molecular weight is expressed as daltons (Da), kiloDaltons (KDa) and megaDaltons, which is million daltons or (MDa). The acronym Mw stands for weight average molecular weight, Mₙ is the number average molecular weight of a given polymer. Polydispersity is a unit-less number and indicates the breadth of the distribution of the polymer molecular weights and is defined as the MW/Mn.

The term “about” is understood to mean±10 percent of the recited number, numbers or range of numbers.

The term “about 0 wt %” is understood to mean that no substance, compound or material to which zero (0) refers is present, up to a negligible but detectable amount is present, assuming that the detectability can be determined on a parts per million basis.

In addition, where features or aspects of the invention are described in terms of Markush groups, those skilled in the art will recognize that the invention is also thereby described in terms of any individual member or subgroup of members of the Markush group. For example, if X is described as selected from the group consisting of methyl, ethyl or propyl, claims for X being methyl and claims for X being methyl and ethyl are fully described. Moreover, where features or aspects of the invention are described in terms of Markush groups, those skilled in the art will recognize that the invention is also thereby described in terms of any combination of individual members or subgroups of members of Markush groups. Thus, for example, if X is described as selected from the group consisting of bromine, chlorine, and iodine, and Y is described as selected from the group consisting of methyl, ethyl, and propyl, claims for X being bromine and Y being methyl are fully described.

If a value of a variable that is necessarily an integer, e.g., the number of carbon atoms in an alkyl group or the number of substituents on a ring, is described as a range, e.g., 0-4, what is meant is that the value can be any integer between 0 and 4 inclusive, i.e., 0, 1, 2, 3, or 4. Similarly, values expressed in a range format should be interpreted in a flexible manner to include not only the numerical values explicitly recited as the limits of the range, but also to include all the individual numerical values or sub-ranges encompassed within that range as if each numerical value and sub-range were explicitly recited. For example, a range of “about 0.1% to about 5%” or “about 0.1% to 5%” should be interpreted to include not just about 0.1% to about 5%, but also the individual values (e.g., 1%, 2%, 3%, and 4%) and the sub-ranges (e.g., 0.1% to 0.5%, 1.1% to 2.2%, 3.3% to 4.4%) within the indicated range.

Hair and hair strands mean natural keratinous surfaces and the term keratinous surfaces means natural keratin fibers which are hair and hair strands. Hair, hair strands and keratin material and keratinous surfaces are used interchangeably in this document and therefore are synonyms. Natural keratinous surfaces include those from mammals and/or on mammals including human, primate, ruminant, camelid, equine, rodent and neovison including but not limited to cow, sheep, deer, goat, buffalo, lama, alpaca, camel, guanaco, vicuna, horse, antelope, moose, elk, rat, mouse, beaver, rabbit, mink, monkey, ape and similar species. Natural keratin material may include hair.

As used herein, the terms “covalent, coordinate, electrostatic, ionic, dipolar and entanglement or entwining interactions” mean a chemical relationship between two atoms or two groups of atoms. The interaction includes a covalent bond between the atoms such as the covalent bond between the two carbons of ethane. The interaction includes a coordinate bond between two or more atoms such as the coordinate bond between oxygen and sulfur of the sulfate anion (SO₄⁻²) or a complex of zinc and EDTA. The interaction includes an electrostatic or ionic interaction between two charged atoms or particles such as the interaction between sodium and chloride of salt or between ammonium and acetate of ammonium acetate. Dipolar interaction includes hydrogen bonding such as the interaction between water and the hydroxyl of methyl alcohol. The interaction includes entanglement or entwining which is lipophilic interaction or mechanical/physical twisting together such as is present in the molecules of polyethylene.

Adherence as used herein generally refers to an arrangement in which a substance formed of a polymer, oligomer or small molecule exhibits a connective aspect with another material such as another polymer, oligomer, small molecule, keratin protein, through such forces as covalent bonding, hydrogen bonding, dipolar interaction, molecular entanglement, mechanical interaction as may be exhibited on a molecular level by a molecular chain wrapping around irregular terrain features of a surface. Adherence in this context may be but not necessarily shown by the inability of the adhered material to be removed from the substance without exertion of any force.

Entanglement as used herein generally refers to an arrangement in which a chain crosses an arbitrary plane 3 times. The chain is then entangled. If the chain is shorter and crossed only two times, it can be pulled in the middle and both ends will release without being bound. With three crossings, if the chain is at one point, it will trap another polymer chain at a different place.

As used herein, the term “transfer resistance” generally refers to the quality exhibited by compositions that are not readily removed by contact with another material, such as, for example, an item of clothing or the skin. Transfer resistance can be evaluated by any method known in the art for evaluating such transfer. For example, transfer resistance of a composition can be evaluated by the amount of product transferred from a wearer to any other substrate after the expiration of a certain amount of time following application of the composition to the hair. The amount of composition transferred to the substrate can then be evaluated and compared. For example, a composition can be transfer resistant if a majority of the product is left on the wearer's hair. Preferably little or no composition is transferred to the substrate from the hair.

As used herein, the term “minimally alters the keratin material or fibers, upon application” generally means that after removal of the composition coating on the keratin material such as hair, the keratin material are returned to a substantially unaltered state. The state of the keratin material such as hair can be assessed for example using ATR FT-IR for oxidative damage as described later or through tensile testing methods known to those skilled in the art for assessing fiber strength for example using equipment such as those designed and sold by Dia-Stron™.

As used herein, the term “curing” means converting the multicomponent composition to a solid coating through the application of means designed to remove or otherwise separate the medium from the other constituents of the multicomponent composition and to cause the covalent bonding of the complementary reactive pairs so as to leave a solid coating.

As used herein, the term “drying” means converting a composition of a preformed polymer and/or a non-reactive polymer in medium to a solid film or coating of the preformed polymer and/or non-reactive polymer by evaporating or otherwise removing the medium. The film produced preferably is an elastomer, exhibits tensile strength and is a substantially homogeneous solid mixture of components.

“Aliphatic substituent, group or component” refers to any organic group that is non-aromatic. Included are acyclic and cyclic organic compounds composed of carbon, hydrogen and optionally of oxygen, nitrogen, sulfur and other heteroatoms. This term encompasses all of the following organic groups except the following defined aromatic and heteroaromatic groups. Examples of such groups include but are not limited to alkyl, alkenyl, alkynyl, corresponding groups with heteroatoms, cyclic analogs, heterocyclic analogs, branched, dendritic, star or fullerene-like and linear versions and such groups optionally substituted with functional groups, as these groups and others meeting this definition of “aliphatic” are defined below.

“Aromatic substituent, group or component” refers to any and all aromatic groups including but not limited to aryl, aralkyl, heteroalkylaryl, heteroalkylheteroaryl and heteroaryl groups. The term “aromatic” is general in that it encompasses all compounds containing aryl groups optionally substituted with functional groups (all carbon aromatic groups) and all compounds containing heteroaryl groups optionally substituted with functional groups (carbon-heteroatom aromatic groups), as these groups and others meeting this definition of “aromatic” are defined below.

As used herein, the term “optionally” means that the corresponding substituent or thing may or may not be present. It includes both possibilities.

“Alkyl” refers to a straight or branched, dendritic, star or fullerene-like or cyclic hydrocarbon chain group consisting solely of carbon and hydrogen atoms, unless otherwise specifically described as having additional heteroatoms or heterogroups. The alkyl group contains no unsaturation, having from one to twenty four carbon atoms (e.g., C₁-C₂₄ alkyl). Whenever it appears herein, a numerical range such as for example but not limited to “1 to 24” refers to each integer in the given range; e.g., “1 to 24 carbon atoms” means that the alkyl group may consist of 1 carbon atom, 2 carbon atoms, 3 carbon atoms, etc., up to and including 24 carbon atoms, although the present definition also covers the occurrence of the term “alkyl” where no numerical range is designated. In some embodiments, it is a C₁-C₄ alkyl group. In other instances it is a C₁-C₆ alkyl group and in still other instances it is a C₁-C₂₄ alkyl group. Typical alkyl groups include, but are in no way limited to, methyl, ethyl, propyl, isopropyl, n-butyl, iso-butyl, sec-butyl isobutyl, tertiary butyl, pentyl, isopentyl, neopentyl, hexyl, septyl, octyl, nonyl, decyl, and the like. The alkyl is attached to the rest of the molecule by a single bond, for example, methyl (Me), ethyl (Et), n-propyl, 1-methylethyl (iso-propyl), n-butyl, n-pentyl, 1,1-dimethylethyl (t-butyl), 3-methylhexyl, 2-methylhexyl, and the like.

“Alkylenyl” refers to a straight or branched, dendritic, star or fullerene-like divalent hydrocarbon chain consisting solely of carbon and hydrogen atoms, unless otherwise specifically described as having additional heteroatoms or heterogroups. The alkylenyl group contains no unsaturation has a valence bond at either end of the chain and has a numerical range of carbon atoms of 1 to 24, which numerical range includes each integen in the range. An example of a divalent hydrocarbon chain designated as an alkylenyl group is —CH₂—CH₂—CH₂—CH₂—

in which the dashes (—) indicate valence bonds to other atoms not shown which is butylenyl.

“Cycloalkyl” is a subcategory of “alkyl” and refers to a monocyclic or polycyclic group that contains only carbon and hydrogen, and may be saturated, or partially unsaturated. Cycloalkyl groups include groups having from 3 to 24 ring atoms (i.e., C₃-C₂₄ cycloalkyl). Whenever it appears herein, a numerical range such as but not limited to “3 to 24” refers to each integer in the given range; e.g., “3 to 24 carbon atoms” means that the cycloalkyl group may consist of 3 carbon atoms, etc., up to and including 24 carbon atoms. In some embodiments, it is a C₃-C₈ cycloalkyl group. In some embodiments, it is a C₃-C₅ cycloalkyl group. Illustrative examples of cycloalkyl groups include but are not limited to the following moieties: cyclopropyl, cyclobutyl, cyclopentyl, cyclopentenyl, cyclohexyl, cyclohexenyl, cycloseptyl, cyclooctyl, cyclononyl, cyclodecyl, norbornyl, and the like.

“Alkoxy” refers to the group —O-alkyl, including from 1 to 24 carbon atoms of a straight, branched, dendritic, star or fullerene-like, cyclic configuration and combinations thereof attached to the parent structure through an oxygen. Examples include methoxy, ethoxy, propoxy, isopropoxy, cyclopropyloxy, cyclohexyloxy and the like. “Lower alkoxy” refers to alkoxy groups containing one to six carbons. In some embodiments, C₁-C₄ alkyl is an alkyl group which encompasses both straight and branched, dendritic, star or fullerene-like chain alkyls of from 1 to 4 carbon atoms.

“Amino” or “amine” refers to an —N(R^{a})₂ group, where each R^{a} is independently hydrogen or linear, branched, dendritic, star or fullerene-like or cyclic alkyl of 1 to 6 carbons. When an —N(R^{a})₂ group has two R^{a} groups other than hydrogen, they can be combined with the nitrogen atom to form a 4-, 5-, 6-, or 7-membered ring.

“Aryl” refers to a conjugated pi ring or multiple rings with six to twenty two ring atoms. The aryl group has at least one ring having a conjugated pi electron system which is carbocyclic (e.g., phenyl, fluorenyl, naphthyl and anthracenyl). Included are partially saturated aryl rings such as tetrahydro naphthyl.

“Heteroalkyl” “heteroalkenyl” and “heteroalkynyl” include optionally substituted alkyl, alkenyl and alkynyl groups and which have one or more skeletal chain atoms selected from an atom other than carbon, e.g., oxygen, nitrogen, sulfur, phosphorus or combinations thereof. A numerical range may be given, e.g. C₁-C₂₄ heteroalkyl which refers to the chain length in total, which in this example may be as long as 24 atoms long. For example, a —CH₂OCH₂CH₃ group is referred to as a “C₄” heteroalkyl, which includes the heteroatom center in the atom chain length description. Connection to the rest of the molecule may be through either a heteroatom or a carbon in the heteroalkyl chain.

“Heteroaryl” or heteroaromatic refers to a 5, 6 or 10-membered aromatic group (e.g., C₅-C₁₃ heteroaryl) that includes one or more ring heteroatoms selected from nitrogen, oxygen and sulfur, and which may be a monocyclic, bicyclic, tricyclic or tetracyclic ring system or a conjugated ring system such as cyclopentadienyl optionally with a bridging atom providing conjugation such as pyrrole or ferrocenyl. Whenever it appears herein, a numerical range refers to each integer in the given range. An N-containing “heteroaromatic” or “heteroaryl” moiety refers to an aromatic group in which at least one of the skeletal atoms of the ring is a nitrogen atom. The polycyclic heteroaryl group may be monocyclic or non-monocyclic. The heteroatom(s) in the heteroaryl group is optionally oxidized. One or more nitrogen atoms, if present, are optionally quaternized. The heteroaryl is attached to the rest of the molecule through any atom of the ring(s). Examples of heteroaryls include, but are not limited to pyrrolyl, furanyl, thiophenyl, imidazolyl, pyranyl, pyridinyl, pyrimidinyl, benzimidazole, benzothiophenyl, quinolinyl, quinazolinyl, and similar heteroaryl compounds of 6 to 12 carbons and 1, 2 or 3 heteroatoms including any combination of nitrogen, oxygen and sulfur.

“Heterocyclic” refers to any monocyclic or polycyclic moiety comprising at least one heteroatom selected from nitrogen, oxygen and sulfur. As used herein, heterocyclyl moieties can be aromatic or nonaromatic. The moieties heteroaryl and heterocyclyl alkyl are members of the heterocyclic group.

The terms “In situ linking” and “in situ linkable” and “Cross linkable” mean the potential at a future time to form covalent bonds, coordinate linkages, ionic linkages, electrostatic linkages, polar couplings, hydrogen bonds and polymer entanglement to provide interactions and/or connections between molecules. The terms “in situ linked” and “cross linked” mean that in the present state, covalent bonds, coordinate linkages, ionic linkages, electrostatic linkages, polar couplings, hydrogen bonds and entanglement arrangements have already occurred.

“in situ” is a Latin phase meaning in its original place. In the context of this invention, it means an activity such a cross linking that takes place on the hair.

The average reactive functional group equivalent weight as used herein means for a reactive functional group of a complementary pair, the ratio of the weight average molecular weight of the polymer, oligomer or small molecule containing the reactive functional group to the average number of occurrences of that reactive functional group in the polymer, oligomer or small molecule. If the Mw of a polymer is 1 KDa and the average number of occurrences of the reactive functional group in the polymer is 2, the Mw for the reactive functional group equivalent weight is (1 KDa)/2 or 500 Da.

Coefficient of thermal expansion refers to the fractional increase in length of a species per Celsius increase in temperature at a constant pressure with a starting temperature of 25° C.

Computer-assisted determination refers to the use of a computer to assist in the choosing or determination of a hair care product that will provide good performance on the given persons hair.

Zeta potential relating to pigment microparticles means the electrokinetic potential of extremely small particles suspended in colloidal dispersions. It is caused by the net electrical charge at the particle interface with the suspending fluid. It is an indicator of the stability of a colloidal dispersion. The magnitude indicates the degree of electrostatic repulsion between adjacent similar charged particles in a dispersion. At zero or minimal + or − potential, rapid coagulation can occur. At a + or − zeta potential above about 40, good colloidal stability is maintained. Zeta potential can be measured using approaches known to those skilled in the art. For example a Zetasizer Nano Z from Malvern Panalytical Ltd, Malvern U.K. may be used to assess the zeta potential of the components.

Microfibril length as used herein general refers to a distribution of lengths for any given microfibril, and the fiber length refers to the average fiber length, assessed over a minimum of 10 fibers chosen randomly from a sample of the microfibrils. The length refers to the end to end distance along the major axis of the material and is not a measure of the cross sectional width.

Hansen Solubility Parameters constitute a technique for characterizing solubility, dispersion, diffusion, chromatography and related topics for a particular material. The material such as a solvent or solute can be characterized by three parameters δD for Dispersion (van der Waals), δP for Polarity (related to dipole moment) and δH for hydrogen bonding. See “Hansen Solubility Parameters—A User's Handbook”, CRC Press, Boca Raton, 2007, ISBN-10: 0849372488.

Hydrogen bonding refers to a weak bond between two molecules resulting from an electrostatic attraction between a proton in one molecule and an electronegative atom in the other. Ionic bonding refers to a type of chemical bonding that involves the electrostatic attraction between oppositely charged ions.

Young's modulus, or the Young modulus, is a mechanical property that measures the stiffness (e.g., stretchness) of a solid material. It defines the relationship between stress (force per unit area) and strain (proportional deformation) in a material in the linear elasticity regime of a uniaxial deformation, e.g., stretch till break point. In other words, the ability of a material to withstand changes in length when under lengthwise tension or compression.

The term ultimate compression refers to the amount of compression a given material can experience under a specific test method before failure occurs and the material breaks.

A nanoemulsion is a liquid/liquid, liquid/solid, liquid/gas or gas/gas composition in which there is at least one discontinuous phase dispersed in a continuous phase and in which the average particle or micellar diameter of the discontinuous phase is in the range 10 nm-300 nm.

Sebum, is an oily, waxy substance produced by the sebaceous glands of the human body. It coats, moisturizes, and protects skin and hair. Sebum is primarily composed of triglycerides (≈41%), wax esters (≈26%), squalene (≈12%), and free fatty acids (≈16%).

The term “surface energy” quantifies the disruption of intermolecular bonds that occurs when a surface is created. The surface energy may be defined as the excess energy at the surface of a material compared to the bulk, or it is the work required to build an area of a particular surface. Perhaps the most widely used definition of surface energy, historically, is that of Zisman (“Relation of Equilibrium Contact Angle to Liquid and Solid Constitution”, W. A. Zisman, ACS Advances in Chemistry Series #43, 1961, pp. 1-51.). Zisman defines the surface energy of a solid to be equal to the surface tension of the highest surface tension liquid (real or imaginary) that will completely wet the solid, with a contact angle of 0°. This comes from the widely observed tendency of contact angle to decrease as liquid surface tension decreases on the same solid sample. A Zisman plot is created for a test surface using a series of different probe liquids with known surface tensions, with the known surface tensions plotted on the x axis, and the cosine of the resulting contact angle with the test surface plotted on the y axis. The highest surface tension when the cosine of the contact angle reaches 1 is determined to be the surface energy of the test substrate. The Owens/Wendt theory (Owens, D. K.; Wendt, R. C.; Jour. of Applied Polymer Science, 13, 1741, (1969)) is a further development to measure the surface energy of a test substrate. It considers the surface energy being comprised of two components—a dispersive component and a polar component. The dispersive component accounts for van der Waals and other non-site specific interactions that a surface is capable of having with applied liquids. The polar component theoretically accounts for dipole-dipole, dipole-induced dipole, hydrogen bonding, and other interactions which a surface is capable of having with applied liquids. Owens and Wendt developed a two parameter model for describing surface interactions, as opposed to the one parameter model of Zisman. The units of surface energy are mNm⁻¹.

The term “textile” as used herein has its ordinary and customary meaning and includes cloth, fabric or other material made out of natural plant fibers, synthetic fibers, metal fibers, carbon fibers, animal fibers such as may be derived from feathers, sinew, ligament, muscle and/or bone. The fibers are combined by weaving, felting, gluing, tacking, spinning, extruding, blowmelting or other-wise formed into at least a somewhat coherent mass typically considered to be cloth, fabric, sponge rubber, foam, woven or nonwoven material. Rugs, bedsheets, clothing, coats, hats, underwear, socks, seat covers, seat cushions, pillows, and similar materials are textiles. Included also is paper made of plant or synthetic material such as typing paper, writing paper, foil, parchment papers, wax paper, aluminum foil and similar flat, thin materials.

### DETAILED DESCRIPTION

The present invention is directed to downstream properties of color coatings on keratin fibers that contribute to, enhance and promote qualities of the coatings so that the coated keratin fibers demonstrate a performance similar to the performance of young, uncoated, vibrant, attractive keratin fibers preferably of the scalp. To accomplish this aspect, the color coatings exhibit qualities including but not limited to flex, stretch and bend, integrity of continuous film under flex, stretch and bend conditions, wash-fastness, remanence, color coordination and resistance to degradation and damage caused at least by, but not limited to, UV radiation, sebum, sweat, rain, wind and/or environmental attack.

The development and preservation of these qualities may be accomplished by one or more combinations of polymer films formed from one or more of preformed color compositions, reactive-polymer color compositions, pre-treatment compositions and post-treatment compositions. These color coatings on keratin fibers exhibit properties including but not limited to one or more characterization ranges for elastomeric character, hardness and tensile strength, surface adherence, coating or film thickness or depth. These color coatings on keratin fibers also exhibit surface qualities including but not limited to non-stickiness, minimal bulk, free hair strand movement, tactile sensation similar to untreated, unbleached human hair, and aural sensation known as the squeak sensation. The color compositions when dressed onto keratin fibers exhibit a combination of entanglement, hydrogen bonding, dipolar interaction and Van der Waals interactions, contact angle, surface tension and surface free energy that enable formation of the color coatings on the keratin fibers that exhibit these properties. The color coatings and the color compositions forming them also preferably include triggers for coating removal.

According to embodiments of the invention, the color coatings on keratin fibers comprise characteristics including their elastomer quality as the Young's modulus, tensile strength, film integrity during flex, remanence against aqueous detergent wash, sebum resistance and resistance to flaking and cracking as indicated by the bend to crack parameter and cross-sectional density and dimension. Irrespective of the kind of polymer and/or polymers comprising the color coating, these parameters measure the flexibility, strength and endurance of the color coating. These parameters may be determined for the color coatings on a swatch of natural, unbleached natural white human hair or bleached natural white hair and for some determinations the color coatings may be formed as a releasable film on a substrate so that the standalone film may be examined. Accordingly, the elastomeric quotient of the color coating as a standalone film may be expressed as Young's modulus in MPa and may be from 1.5 to 5000 MPa, preferably 3 to 2000 MPa, more preferably 5 to 250 MPa wherein a higher Young's modulus indicates a stiffer material. The tensile strength of the color coating as a standalone film may be from 0.5 to 300 MPa which measures the strength or tension required to break or snap a coating film by application of a pull force to the opposite ends of the film. The film integrity may be measured by number of flexes to 60% of its ultimate elongation of a standalone film of color coating. The film integrity before breakage may be at least 50 flexes, preferably at least 150 flexes, more preferably at least to 300 flexes. An adhesion integrity of the colored coating may be determined upon a color coating applied and affixed to the natural white hair swatch and/or bleached natural white hair swatch. The measurement is determined by flexing the color coated swatch to at least 600 for at least 150 repeats over a period of at least 10 minutes. Suitable adhesion integrity is determined by visual and weight examination of debris. Visual debris from the flexed color coated swatch may be no more than barely visible debris. The weight of the debris should be no more than about 2 to 4 weight percent of the total weight of the color coated swatch before flexing. The abrasion of the film may also be determined using a Crockmeter (Crockmeter Typ CRO-B-P, Werner Mathis AG). A coated hair tress is secured within the device at both ends, and using a standard white cotton cloth (Werner Mathis AG testing cloth (50×50 mm) according to norm ISO 105-F09 or NBR 8432) on the moving portion with normal force of 9 N the cloth is moved backwards and forwards each cycle. This can be assess after 30 cycles, 50 cycles or 150 cycles. The color on the cloth and or the tress is then assess colormetrically using a Minolta CM2600d. The color of cloth is preferably less than a de2000 of 40, more preferably less than 20, even more preferably less than 10, most preferably less than 5 of the starting cloth color. The color of tress is preferably less than a de2000 of 10, more preferably less than 7, even more preferably less than 5, most preferably less than 2 of the starting coated hair tress.

The remanence and sebum resistance of the color coating are determinations of the ability of the colored coating to adhere to the keratin fibers under adverse conditions, more specifically. avoidance of dislodgement under conditions of shampooing, brushing, washing and rinsing and contact by sweat and sebum. These are measured by the number of detergent washes and sebum formulation brushing required to cause a decrease in color intensity of the color coating and/or a decrease in cross sectional dimension of the coating. The minimum remanence of a color coating according to the invention dressed on a natural blond hair swatch coating may be determined by washing the color coated swatch at least 5 more preferably 10 times, even more preferably 15 times most preferably 30 times using a shampoo (Wella Professionals INVIGO Color Brilliance Shampoo for Coarse Hair). 0.1 g of shampoo is applied to 1 g of pre-wetted hair, lathered for 30 seconds and then rinsed in water at 37+−2° C. at 4 L min for 30 seconds in wash cycle follow by drying the hair. According to the invention, the minimum remanence determined by this procedure may be preservation of at least 80 percent of the initial swatch color intensity, measured dE2000 versus the uncolored hair tress and/or at least preservation of at least 80 percent of the cross-sectional dimension of the coating. The sebum resistance of the color coating may be measured also on such natural white hair swatch and/or bleached natural white hair swatch coated with the color coating according to the invention. 5 sebum cycles are performed in the testing. Each cycle comprises the following steps. 0.1 g of synthetic sebum is applied to the hair tress, and distributed with gloved hands through the hair swatch. The tress is then left in a 40° C. oven for 30 minutes. The tress is then washed with one shampoo wash as described above. This process is repeated for a total of 5 treatments and the color measured. The minimum sebum resistance of a color coating according to the invention may be preservation of at least 80 percent of the initial swatch color intensity, measured by dE2000 versus the uncolored hair tress and/or at least preservation of at least 80 percent of the cross-sectional dimension of the coating.

According to the invention, the environmental challenge by UV radiation, wind, and heat attack and brushing may also be determined by the colored coating swatch method. UV testing is performed by treating the swatch for 12 h of irradiation per strand side, i.e. 24 hours across both sides using irradiation power: 50 W/m2 in an Xenochrome 300 with a Supraxfilter to simulate sunlight. The samples are 8 cm from the lamp (and turn around the lamp). The starting temperature in the chamber is 35° C. and the temperature of the chamber during testing does not exceed 67° C. when an auto shut down would occur. Relative humidity was between 40% and 80%. The wavelength of the light irradiation on the tress is between 300-800 nm For each challenge, the colored coating swatch is subjected to the chosen attack for at least 20 exposures. Wind and heat attack may be simulated by a hot air hair dryer for 60 minutes . . . . Exposure of the colored coating swatch to repeated brushing is performed with a stiff bristle brush at a rate of ten to fifteen strokes per minute over a 10 to 15 minute period. For all environmental challenges, the minimum resistance of a color coating according to the invention may be preservation of at least 80 percent of the initial swatch color intensity, measured by dE2000 versus the uncolored hair tress and/or at least preservation of at least 80 percent of the cross-sectional dimension of the coating

According to the invention, the surface qualities of the color coatings on keratin fibers may be determined by color coating swatch method. These surface qualities include but are not limited to non-stickiness, minimal bulk, free hair strand movement, tactile and aural sensations. Non-stickiness, minimal bulk free hair strand movement and tactile and visual sensations of the color coated swatch may be determined by comparison of the tactile and visual sensations of a standard untreated hair swatch and the candidate hair swatch with colored coatings. The tactile and visual sensations produced by feeling with finger rubs and visual inspection of standard and candidate at rest and standard humidity, temperature and environmental factors. The tactile and visual sensations that may be produced by the candidate may be within at least 80 to 90 percent of the sensations produced by the standard while making allowance for the differing color and increased weight of the candidate. The standard and candidate are then placed in a wind chamber and subjected to environmental challenge by exposure to increasing degrees of humidity, temperature and air flow over a period of 1 hour. At 10 minute intervals, the environmental challenge is ceased and the tactile and visual sensations produced by the standard and candidate are compared. The tactile and visual sensations that may be produced by the candidate throughout the environmental challenge may remain within at least 80 to 90 percent of the sensations produced by the standard while making allowance for the differing color and increased weight of the candidate. The free hair strand movement may be assessed by observing the performance of the standard and candidate in the wind chamber during increasing humidity, temperature and air flow. The candidate may exhibit hair strand movement within at least 75 to 80 percent of that exhibited by the standard. The hair strand movement may be rated by raise angle of the strands relative to a vertical at rest position and by the number of strands moving independently relative to the number of strands visually moving together due to inter-strand attachment.

Another aspect of the invention is directed to the technique and features for dressing the one or more color compositions onto keratin fibers including but not limited to hair on the scalp of humans. The one or more color compositions including but not limited to the pre-treatment composition, the preformed polymer composition, the reactive-polymer composition and the post-treatment composition are compositions demonstrating flowability before dressing onto the keratin fibers. Flowability is accomplished through mixture of the liquid media component with the solid and/or gel, and/or liquid components of the one or more color compositions that remain after removal of the liquid media. The flowability is measured by the viscosity, contact angle, surface tension and surface free energy of the one or more color compositions before and during dressing onto the keratin fibers. Additional features for dressing of the one or more color compositions onto keratin fibers include the rate at which the liquid media may be removed after dressing such as but not limited to evaporation, the rate of contiguous film formation of the one or more preformed polymers, the pre-treatment polymers and the coupled, cross linked polymer products formed from the reactive-polymers. An additional feature for dressing of the reactive-polymers is that timing and rate for coupling of the complementary reactive groups of the reactive-polymer pairs.

The flowability and rates of film formation and coupling are adjusted by concentrations, temperatures, liquid media removal parameters such as but not limited to evaporation rate so that dressing the one or more color compositions onto keratin fibers may be accomplished in an unhurried manner. The viscosity of the one or more color compositions may range from about 1 to about 50 mPa s (millipascal-seconds) so that the one or more color compositions have a viscosity like whole milk (low viscosity) to olive oil (high viscosity). Preferably, the one or more color compositions include an agent that can confer thixotropy. Preferably the one or more color compositions are viscous like honey at rest but become significantly less viscous upon agitation. This preferable thixotropy enables the one or more color compositions to be applied to keratin fibers in readily flowable condition but once applied, do not run off.

The contact angle and surface free energy of the one or more color compositions cooperate with viscosity to enable appropriate dressing of the one or more color compositions onto keratin fibers. The contact angle and surface free energy are at least in part determinative of the ability of the one or more color compositions to form contiguous one or more color compositions on the keratin fibers during dressing and before removal of the liquid media and/or before curing of the reactive-polymers. The contact angle of the one or more color compositions at this stage of dressing may be at most 200 and preferably as close to 0° as possible on the hair surface. The surface free energy of the one or more color compositions at this stage of dressing may be 24 to 50 mN m⁻¹, in other words, similar to ethanol and lower than water. The low surface free energy enables rapid and contiguous spreadability of one or more color compositions at this stage of dressing. A low contact angle is the result also indicating high spreadability. The low surface free energy may be accomplished by inclusion of surfactant in the one or more color compositions if the spreadability of the one or more color compositions does not provide contiguous coverage. Control of the thixotropy so that reversion from a thin free flowing fluid to a viscous material occurs over a period of at least 5 minutes. Also, the thixotropic character enables the re-distribution of the one or more color compositions by stroking with a very fine bristle or finger brush or similar applicator. The stroking engages the thixotropic character of the one or more color compositions enabling the composition or compositions to flow on the keratin fibers after initial dressing.

### Formation of Color Coating

Embodiments of the color compositions according to the invention comprise the pre-treatment composition, the preformed polymer composition, the reactive-polymer composition, the composition properties, the dispersant composition, the additives composition, the post-treatment topcoat, the care composition and the removal composition. One or more or all of the pre-treatment, preformed polymer, reactive-polymer composition and the topcoat composition may contain pigment particles. Any of the compositions containing pigment will also contain a dispersant composition. While one of the color composition embodiments may be dressed alone onto keratin fibers, it is preferred to dress the keratin fibers first with the pre-treatment composition followed by either or both of the preformed polymer composition and the reactive-polymer composition. The pre-treatment composition incorporates a base compound comprising an organic, silicone and/or organosilicone polymer or a silane monomer, dimer, trimer, tetramer, pentamer or hexamer containing amine groups as pendant groups and/or as groups in the polymer chain or small molecule. The base compound of the pre-treatment composition develops non-covalent bonding to the keratin fibers. The base compound of pre-treatment composition also provides a coating layer promoting adherence of the preformed polymer and/or reactive-polymer compositions onto the keratin fibers. The adherence is at least in part responsible for the tensile strength, film to fiber connection and remanence of the color coating on keratin fibers.

The non-covalent bonding or adherence of the base compound of the pre-treatment composition to the keratin fibers occurs through ionic, electrostatic and dipolar interactions between the its amine groups and the carboxyl and carboxamide groups of the keratin proteins of the fibers. Adherence is also the result of molecular intertwining between the amine polymer chains and the irregular surfaces of the keratin fibers. The non-covalent bonding is preferably accomplished by an Mw of the base compound as a polymer of from 5 KDa to 1 MDa. The base compound as an amine polymer preferably includes an in-chain amine group or a pendant amine group for every or almost every monomeric residue of the polymer. Exemplary amine group distributions are shown but not limited to that shown by polyethyleneimine (PEI) and poly (methyl, (3-aminopropyl)methyl) silicone. Branching and cross-linking of the amine polymer provides a loose network configuration of the three-dimensional polymer structure. The base compound may alternatively be a silane small molecule as a monomer, dimer, trimer, tetramer, pentamer or hexamer having a distribution of amine groups and hydroxy and/or alkoxy groups. The Si-alkoxy and Si—OH groups enable the small molecule to condense to form longer Si—O—Si chains in situ. The presence of amine groups within the silane small molecule provides the adherence of the small molecule and its condensed version to the keratinous surfaces.

The polymer of the preformed polymer composition and polymer product formed by the reactive-polymer composition are organic, silicone and/or organosilicone polymers modified by in chain and pendant groups that provide hydrogen bonding, dipolar interaction and chain entanglement as well as by cross linking groups that establish branching, network, star and combinations thereof for the microstructures of the polymers. The adherence quotient of these polymers is determined by a combination of physical and chemical factors of the polymers including weight average molecular weight (Mw), dispersity, extent of cross-linking, entwining or entanglement of polymer chains, extent of chain branching and equivalent Mw's for cross-linking, entanglement and extent of chain branching as well as extent of hydrogen bonding, dipolar interaction and van der Waals forces. Irrespective of the structural identities of individual polymers, the combination of these physical and chemical factors provides the adherence quotient for at least in part attaining elastomer, tensile strength, remanence, wash-fastness and resistance to environmental attack. The Mw of the preformed polymer is in a range of about 5 KDa to about 1 MDa, preferably about 10 KDa to about 500 KDa, more preferably about 20 KDa to about 250 KDa. The Mw of the in situ polymer product resulting from reaction of the complementary groups of the reactive-polymer pairs is at least 5 KDa and may range to almost infinity due to the cross-linked network configuration of the in situ polymer product. The preformed polymer and the polymer product from the reactive-polymer pairs may comprise hydrogen bonding and dipolar interaction groups including urethane, urea, ester, amide, β-(amino or hydroxy)ethylenylacyl, β-amino-α-hydroxypropyl, N-acylurea and any combination thereof distributed within the polymer chains. The distribution of the hydrogen bonding and dipolar interaction groups within the polymer chains may comprise an equivalent Mw per hydrogen bonding group of 250 Da to 5 KDa. The preformed polymer and the in situ polymer product from reactive-polymer pairs may also comprise cross-linking moieties formed from olefinic couples, urethane couples, urea couples, ester couples, amide couples, hydroxysilane and alkoxysilane condensation couples, MDTQ siloxane couples, β-(amino or hydroxy)ethylenylacyl couples resulting from Michael addition, β-amino-α-hydroxypropyl couples resulting from epoxymethylenyl addition, N-acylurea couples resulting from carbodiimide addition and any combination thereof distributed between polymer chains. The cross-linking establishes a network and/or star and/or branched microstructures of the polymers. The cross-link density for the pre-formed polymer and the polymer product from the reactive-polymer pairs may range from at least one cross link per 5 polymer chains to at least 1 cross link per one, two or three monomeric units of the polymer. The Mw per cross link may range from about 5 KDa to about 50 to 100 Da. The preformed polymer and polymer product from reactive-polymer pairs also include entanglements of polymer chains resulting from lipophilic interaction and “floppy” chain interaction. Entanglements increase as branching, networking and star microstructures increase; however, linear unbranched, non-crosslinked polymers also display entanglements. The equivalent Mw for entanglements of the preformed polymer and the polymer product from the reactive-polymer pairs may be in the range of from about 1 KDa to about 10 KDa. The combination of hydrogen bonding, dipolar interaction, cross linking and entanglements provides the adherence quotient of the polymers of the color composition. The combination equivalent Mw resulting from the combination of equivalent Mw's of each of these factors may be in the range of from about 500 Da to about 5 KDa. Because the entanglement factor will always exist to a greater or lesser degree, it is balanced by the combination the other factors including hydrogen bonding, cross linking, dipolar interaction which can be intentionally controlled by introducing a greater or lesser number of these groups into the preformed polymer and the polymer product formed from the reactive-polymer pairs.

The preformed polymer composition and the reactive-polymer composition may also optionally comprise materials and substances that contribute to the elastomeric, tensile strength, flex and bend durability and wear resistance of the color coating on keratin fibers. These materials and substances include but are not limited to plasticizers, dispersants, synthetic and/or natural clays, synthetic and/or natural microfibers and any combination thereof. The plasticizers and dispersants enable polymer molecules and chains to “slip” past each other under stress conditions resulting from stretching, elongation and compression. The “slip” factor contributes at least in part to the elastomeric quality of the color coating on keratin fibers. The plasticizers and dispersants are compatible with the polymers and are well-known materials for such purpose. They are described in detail in a separate section below.

The synthetic and/or natural clays and microfibers contribute at least in part to the tensile strength, wear resistance and remanence of the color coating on keratin fibers. The clays and microfibers are known for their contribution to protective and wear attributes of coatings in the industrial substrate coatings such as appliance and vehicle coatings. The clays and microfibers enhance smoothness of the color coating on keratin fibers and lessen the tendency toward film (coating) breakage or flaking during elongation and stretching. Minor amounts of clays and microfibers provide sufficient enhancement of these features. The natural clays are aluminum phyllosilicates of kaolinite, montmorillonite, smectite, illite and chlorite classes. The natural clays typically are lamellar in form and are intercalated with magnesium, potassium and calcium ions. Natural clays may be modified by exchanging the intercalated ions with organic molecules capable of accepting a charge. Microfibers are ultrafine synthetic fibers formed from polyester and/or polyamide and/or cellulose and/or polyolefin and produced commercially as Ultrasuede, Primaloft, Prolen, Polar fleece and Microfiber. Microfibers are elastic so that when combined in minor amounts with the color composition embodiments of the invention, they contribute to the elasticity of the color coating on keratin fibers.

Embodiments of the topcoat composition are applied post dressing and curing of the pre-treatment and color compositions. The topcoat is similar to hair styling spray and comprises a polymeric olefinic ester in aqueous organic solvent. The topcoat may also comprise a sacrificial material such as an organic or silicone oil. The sacrificial material protects the dried and/or cured color coating on the keratin fibers by preventing or minimizing penetration of environment attack onto and into the color coating.

### Cross Link and Hydrogen Bonding Analysis for Remanence and Wear Resistance

When the reactive polymer pairs with complementary reactive pairs or with self-reactive functional groups of the reactive-polymer composition are combined such as dressed to hair, they will undergo an in situ covalent linking reaction leading to the formation of new covalent bonds. Similarly, when the preformed polymer composition and the polymer product of the reactive-polymer composition are combined with the amine polymer of the pre-treatment composition, the hydrogen bonding exerts strong interpolymer connections although less than the covalent cross linking. According to the multicomponent features of the invention, application to the substrate material and especially the hair of the many variations of the multicomponent composition results in the formation of a solid, flexible coating having a network and/or star three dimensional configuration. Colored pigment particles are embedded in the coating. The new in situ formed bonds and hydrogen bonds can change the rheological characteristics of the coating relative to the rheological characteristics of the separate reactive polymer pairs and preformed polymers with significant in-chain and pendant hydrogen bonding groups. Whilst not wishing to be bound to any particular theory, it believed to be advantageous if the reactive polymer pairs and hydrogen bonding quotient change from having a substantial G″ component, the so called loss modulus, and a negligible G′ component, the so called storage modulus, prior to reaction and combination such as application to the hair, to the reverse situation where there is negligible G″ component and a substantial G′ component. This can also be considered by consider the phase angle φ, where

$$φ = arc tan ( \frac{G \text{"}}{{G}^{′}} )$$

When the complex shear modulus changing from more the 45 degrees to less than 45 degrees. Both the phase angle of the resulting film or coating, and the complex shear modulus can be optimized for performance. Alternatively, it may be possible to quantify the resulting coating properties in terms of Young's Modulus and elongation at breaking.

The following parameters further help to define the properties of the materials that are particularly useful. Whilst not wishing to be bound by any particular theory, the degree of connectedness, i.e. formation of new in situ connections and the separation between these new connections can impact the performance of the resulting composition on substrate material such as hair. With too many connections and too little separation between the connections the resulting interconnected material can be too stiff, leading to poor performance on hair, both tactile perception and permanence through extended hair washing. This negative performance can also be expressed as having a phase angle φ less than 30 degrees, more particularly less than 15 degrees, even more less than 2 degrees. Conversely if there are insufficient connections and the separation between them is too great, the material is too fluid like leading to sticky feel, a potential for transfer of the color to other surfaces, and lower permanence and resistance to washing. This negative performance can also be expressed as having a phase angle φ greater than 60 degrees, more particularly greater than 75 degrees, even more particularly, greater than 88 degrees.

The average length between new in situ covalent and hydrogen bond connections can be described as the average in situ link length for a given polymer and is the average distance between successive reactive monomeric units and hydrogen bonding units (which are OZ of the following mathematical formula) of a molecule:

$$Cross link length = \frac{100}{{\sum }_{n = 1}^{n = n} MPC {( OZ )}_{n}}$$

for a series of n potential OZ groups within a given polymer and the term MPC is defined as Mole Percent, which is equal to the number of modifications of the given species per 100 monomeric groups within the polymer.

The cross linking role for a given species is given by:

$$Cross link role = \frac{{\sum }_{n = 1}^{n = n} {N}_{n} M P {C}_{n} D P}{200}$$

For a series of n potential reactive and hydrogen bonding units (OZ) within a given polymer where N is the number of OZ groups for the given functional group that can form cross links and hydrogen bonds with other functional groups, MPC is the mole percent of the given group within the polymer and DP is the number average degree of polymerization of the polymer. When present, polymers which do not have any OZ functionality have a cross link role=0, they will not form any new polymer connections. When the cross link role=1, the first and second polymers only perform the role of chain extension when used by themselves, although those skilled in the art would understand that such chain extension would not necessarily have to occur through terminal ends of the polymer chain. When the cross link role >1 the first and second polymers can perform network building, the higher this number the greater the impact of the network building. The properties of the resulting film or coating will depend on a complex relationship of the in situ link length and the in situ link role and dilution roles of all of the constituents of the composition including but not limited to the first and second components of the reactive polymers and any preformed polymers used, if any.

With solid structures that resist solvation, hydrogen bonding can provide a significant connective force. For estimation purposes, hydrogen bonding may be estimated to develop a connective force approximately 5 to 10 percent that of covalent bonds. To calculate the factor attributed to hydrogen bonding, the number of hydrogen bonding groups per cross link equivalent Mw is counted and from 5 to 10 percent of this number times the mass factor contributed by the equivalent cross link molecular portion is added to the mass fraction based on cross linking for the following formula.

Where more than one reactive polymer pair and a preformed non-reactive polymer is used the following factors need to be considered. For each polymer added, the reduced fraction of the given polymer needs to be calculated.

$$Reduced Fraction = \frac{\frac{Mass Fraction component}{D P}}{{\int }_{n = 1}^{n = n} \frac{Mass Fraction {component}_{n}}{D {P}_{n}}}$$

Where the mass fraction of the component is the percent of the polymer phase. If present, silicones which do not have an OZ (Formulas I and II) functionality are also included within the calculation to determine the reduced fraction of the total silicone phase. The DP is the number average degree of polymerization, i.e. the number of monomeric units within the polymer. This effectively factors the number of each type of polymer added by the number of individual polymer entities versus just using the weight of the amount of species added. Thus, the effect of a low DP material, e.g. with a DP=10 can produce a larger effect versus the same addition of a higher DP material, e.g. with a DP of 10,000. When added at equal weights, there are 100 times more of the low DP polymer entities versus the high DP polymer chains.

For the mixed system the following terms can be calculated.

$$Average cross link length = {\int }_{n = 1}^{n = n} Cross linked {length}_{n} \times Reduced {Fraction}_{n}$$

For n polymer materials within the formulation.

$$Average cross link role = {\int }_{n = 1}^{n = n} Cross linked {role}_{n} \times Reduced {Fraction}_{n}$$

For n polymer materials within the formulation.

Using these terms for the polymer phase, non-limiting material combinations which are preferred include those where the average cross link length is greater than 10, more preferably greater than 25, even more preferably greater than 50, and where the average cross link role is greater than 1.3, more preferably greater than 1.5, even more preferably greater than 1.6. Preferably the average cross link length is less than 400, more preferably less than 350, even more preferably less than 250, and where the average cross link role is less than 8, more preferably less than 4.5, even more preferably less than 4.

### Preformed Organic Polymers

The organic polymer of the preformed polymer composition may include but is not limited to linear and/or branched configurations of homopolymer, copolymer, terpolymer and/or embodiments of single and multiple monomeric units polymer embodiments. These embodiments comprise small molecules, oligomers and polymers of appropriate monomeric units such as but not limited to unitary, dimer, trimer, tetramer up to twenty monomeric units, one or more olefin monomers, ester units of diacids/diol monomers, ester units of hydroxy acid monomers, ether monomeric units, thioether monomeric units, polyol monomeric units, alkylene oxide monomeric units, alkylene imine monomeric units, urethane monomeric units urea monomeric units, amide units of diacid/diamine monomers, amide units of amino acid monomeric units, amino acid units providing peptides, gelatin or biopolymers; carbohydrate monomeric units providing alginates, cellulosic derivatives, polysaccharides; as well as other repeating residues based on carbon or carbon in combination with other atoms such as oxygen and/or nitrogen, and any combination thereof. Preferably the oligomers and polymers are polyolefins, polyesters, polyethers, polyurethanes or polyamides or any combination thereof. Also preferred are small molecules with one, two, three up to about 20 ester groups, ether groups, urethane groups, amide groups and any combination thereof. More preferably, the oligomers and polymers are polyolefins, polyesters or polyurethanes or any combination thereof. Especially more preferably, the oligomers and polymers are polyolefins or polyesters. Also especially preferred are hydrophobic film forming polymers incorporating acrylic polymers, polyurethanes, polyesters, polyamides, polyureas, and silicone polymers having a water solubility of no more than one percent by weight of the polymer in water at neutral pH and 25° C. Embodiments of preferred preformed polymers include but is not limited to a copolymer of acrylic acid, isobutyl methacrylate, ethyl methacrylate and t-octyl acrylamide and a copolymer of polyurethane and polyacrylate units of 1,6-dihydroxyhexane, 1,6 diisocyanatohexane, hydroxyethyl acrylate, ethyl methacrylate and acrylic acid.

The organic polymer may have non-polar, non-protic pendant moieties such as but not limited to linear, branched or cyclic alkyl groups optionally including oxygen, nitrogen, ester, oxycarbonyl, amide, hydroxyl, thioether, ether, amino, imino, sulfonyl within or along the alkyl groups. These pendant moieties also include aromatic groups, heteroaromatic groups, small to oligomeric repeating carbon units, all with the same optional heteroatoms and heteroatom groups described for the alkyl chains and/or moieties. These pendant moieties may also be oligomeric or polymeric silicone moieties constructed of organosiloxane units.

The preformed organic polymers can be conceptualized as classes, subclasses and categories of organic polymers without the first functional groups. Such preformed organic polymers include the above described oligomers and polymers. These preformed organic polymers include but are not limited to oligomers and polymers of appropriate monomeric units such as but not limited to one or more olefin monomers, ester units of diacids/diol monomers or of hydroxy acid monomers, ether monomeric units, thioether monomeric units, polyol monomeric units, alkylene oxide monomeric units, alkylene imine monomeric units, urethane monomeric units urea monomeric units, amide units of diacid/diamine monomers or of amino acid monomeric units, amino acid units providing peptides, gelatin or biopolymers; carbohydrate monomeric units providing alginates, cellulosic derivatives, cellulose esters, polysaccharides; hydroxylated polyester, acrylate functionalized polyester, polyester polyurethane acrylic copolymer, polyurethane-polyglycol copolymer, polycarbonate diols, styrene-allyl alcohol copolymer, ketone resins; as well as other repeating residues based on carbon or carbon in combination with other atoms such as oxygen and/or nitrogen, and any combination thereof. Additional preformed organic polymers include but are not limited to non-polar olefinic polymers, polar, non-protonic olefinic polymers, vinyl polymers, polyethers, polycondensates, block polymers and any compound with repeating carbon unit residues. Preferably the preformed organic polymers are polyolefins including polyvinyl compounds, polyesters, polyethers, polyurethanes or polyamides or any combination thereof. More preferably, the organic polymers are polyolefins including polyvinyl compounds, polyesters or polyurethanes or any combination thereof. Especially more preferably, the organic polymers are polyolefins, polyvinyl compounds or polyesters.

Preformed organic polymers containing acid groups may be developed from any monomeric unit containing acid groups such as carboxylic acid, sulfonic acid, sufinic acid, phosphoric acid. The acidic units may be combined with non acidic units which are hydrophilic or hydrophobic to provide appropriate preformed organic polymers. Such polymers are described in the following passages.

Preformed polymers may include copolymers of (meth)acrylic acid and of at least one linear, branched or cyclic (cycloaliphatic or aromatic) (meth)acrylic acid ester monomer and/or of at least one linear, branched or cyclic (cycloaliphatic or aromatic) mono- or disubstituted (meth)acrylic acid amide monomer.

Additional disclosure of preformed polymers is provided by PCT application WO2019/192916 published Oct. 10, 2019 wherein the preformed polymers are characterized as precursor polymers. The disclosure of WO2019/192916 is incorporated herein by reference.

### Preformed Silicone and Organosilicone Polymers

The preformed silicone polymer may include but is not limited to polysilicones as well as organo-silicones of the pendant or graft type as shown by Formula I wherein preformed substituents are incorporated within or onto monovalent organic groups, A₁, A₂ and A₃ and an organic group C which does not contain a preformed substituent. The preformed silicone polymer may contain in any order and in any number the Siloxane Unit Designations (SUD) I, II, III, IV V and VI including preformed silicone polymers of SUD's of I and II alone to provide polysilicones, SUD's of I, II and VI to provide polysilicones with C groups and SUD's of any combination of I, II, III, IV, V and V to provide polysilicones substituted by one or more and any combination of methyl groups and organic groups A₁, A₂ and A₃ and C. SUD I terminates the silicone polymer. The dangling valences of SUD's I, II, III, IV, V and VI are the bonds to the next siloxane unit. Multiples of the SUD's bonded together form the silicone polymer. With this arrangement, the preformed siloxane units SUD III, IV and V may appear anywhere within the polymer and may be interspaced with SUD II which contains only methyl substituents and SUD VI which contains aliphatic, aromatic and heteroaromatic groups. Preformed siloxane units SUD III, IV and V may contain the same or different preformed substituents. Each instance of SUD III, IV and V may be interspersed with other SUD units along the silicone chain. Each instance of SUD III, IV and V may be the same or may be different from any other instance of SUD III, IV and V so that preformed siloxane units of multiple specific identities, of a few different identities or of the same identity may appear throughout the silicone chain. The same dispersion and variation of identity applies to each instance of SUD VI. The substituents A₁, A₂ and A₃ are organic groups with preformed substituents. The substituent C is an organic group with a neutral substituent or an aliphatic, aromatic or heteroaromatic group without a polar substituent. The variable Bi is an SiC organic group as defined above, and preferably is methyl, OH, an alkyl or an alkoxy group wherein the alkyl or alkoxy group is a linear alkyl or alkoxy group of 1 to 6 carbons, or a branched or cyclic alkyl or alkoxy group of 3 to 6 carbons.

Together, SUD III, IV and V constitute SiA preformed units. SiA for Formula I expands the kind of unit from only an amine to any preformed substituent as set forth below. Together, SUD I, II and VI constitute SiC non-functional units. SiC for Formula I narrows the kind of unit included because hydrogen bonding groups and dipolar groups are included within the SiA definition for Formula I.

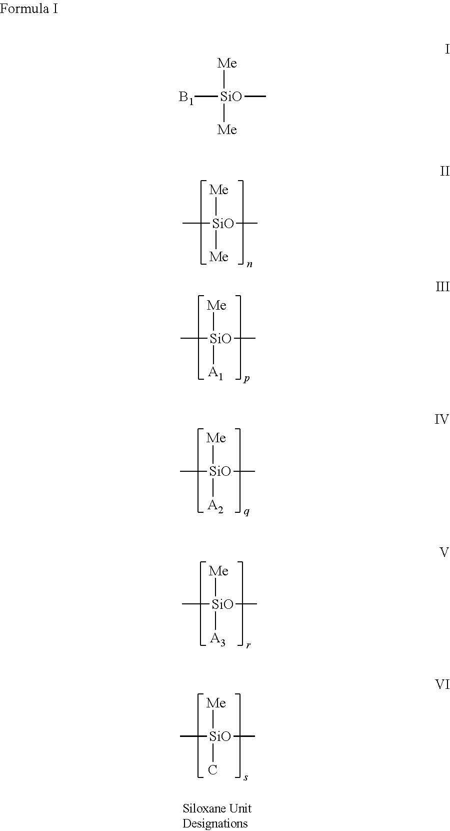

The SUD's of Formula I are present according to certain molar amount ranges. The range n for SUD II is about 50 to 2000. The range p for SUD III is about 0 to 50. The range q for SUD IV is about 0 to 50. The r for SUD V is about 0 to 50, The s for SUD VI is about 0? to 50. At least one of p, q, r and s is greater than 0. The sum of p+q+r is the sum of the number of SUD units III, IV and V. This sum provides the number of SiA units carrying preformed substituents. The sum n+s+2 is the sum of the number of SUD units II, VI and the two termini units SUD I. This sum provides the number of SiC units. Thus, the sum of p+q+r relative to the sum of n+s+2 provides a ratio of siloxane preformed units (SiA) to non-polar siloxane monomeric units (SiC). The ratio of SiA:SiC for Formula I is from about 1:1000 to about 1:3 (moles of SiA to SiC units), preferably 1:1000 to 1:4, more preferably 1:600 to 1:5, most preferably 1:500 to 1:25 or 1:400 to 1:40.

Also included are the organomodified silicones of the block copolymer type as shown in Formula II wherein these preformed substituents are incorporated within or onto organic oligomer moieties. The SUD'S I, II and VI as defined above and as repeated multiple times form the silicone portion of the block copolymer while the Organic Oligomer Unit Designations (OOUD) VII, VIII and IX form the organic polymer block units. The OOUD's are blocks so that OOUD VII, VIII or IX may be present alone one or multiple times in the block copolymer or a mixture of OOUD VII, VIII and IX blocks may be present single or multiple times in the block copolymer. Multiples of the SUD units II and VI may be present as unitary blocks or as mixed units in a block and form the silicone blocks of the copolymer. The copolymer is terminated by SUD I. The Org of OOUD VII, VIII and IX may be large or short oligomeric units of polyolefin, polyester, polyamide, polyurethane, polyol, polyurea and similar organic polymeric groups. The oligomeric units are substituted by organic groups A₁, A₂ and A₃ which carry preformed substituents as defined above. Bi is methyl, OH, an alkyl or an alkoxy group as described above for Formula I.

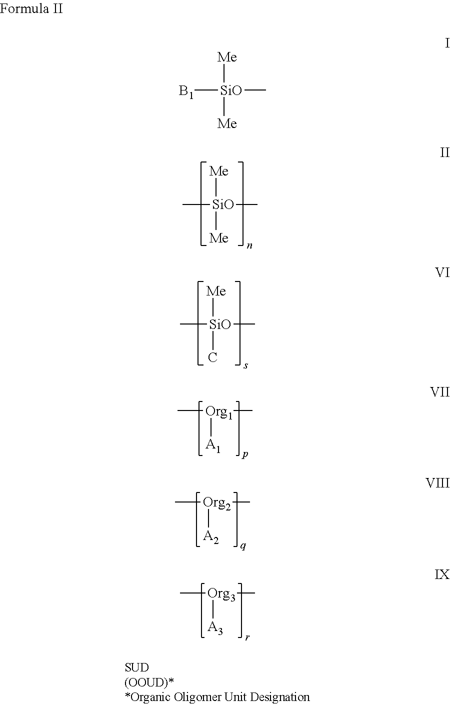

The SUD's and OOUD's divide into two categories. The preformed unit category SiA includes the OOUD's while the non-functional unit category includes the SUD's. The sum of p+q+r provides the total molar number of SiA units while the sum of n+s+2 provides the total molar number of SiC units, wherein the number 2 accounts for the two terminal SUD I units. The total molar number of SiA units is greater than or equal to 1 so that at least one of p, q and r is at least 1. The molar number ranges for each of the SiA OOUD's provides p as about 0 to 50, q as about 0 to 50 and r as about 0 to 50. The molar number ranges for SiC SUDII and OOUD VI provides n as about 50 to 4000 and s as about 0 to 50. The molar ratio of SiA units to SiC units is from about 1:1000 to about 1:10, preferably 1:1000 to 1:25, more preferably 1:600 to 1:50, most preferably 1:400 to 1:75 or 1:300 to 1:200. The preferred Bi group for Formula I and Formula II is methyl, hydroxyl and alkoxy. With Bi as methyl, the terminus of the polysilicone is non-reactive. With Bi as hydroxyl or alkoxy, the terminus of the polysilicone is reactive so that it will possible that they can couple to form longer chains. This coupling will form macropolymers of the polysilicone.

The foregoing preformed pendant or block silicones include siloxanes designated as a D group, Me2Si02/1, i.e, —O—Si(Me)₂-O—. These preformed pendant or block silicones can also incorporate siloxane branching groups and cross linking groups including MeSiO3/2, known as silsesquioxane or T groups, and SiO4/2, known as Q groups by those skilled in the art. The T groups of two silicone chains can combine to form a cross link such as is depicted by Formula III

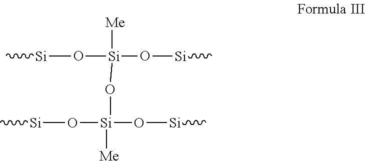

If the T and Q groups are formulated with hydroxyls and mixed with water incident with application to hair, the hydroxyls couple as described above to cross-link. The result will be similar to that described above for the macropolymer formation discussed above.

Organic groups A₁, A₂ and A₃ maybe straight, branched or mono- or polycyclic aliphatic, mono or polyunsaturated alkyl, aryl, heteroalkyl, heteroaliphatic or heteroolefinic moieties or any combination thereof comprising 3 to 150 carbon atoms together with up to 50 heteroatoms and/or heteroatom groups. The heterogroups establish polarity within the preformed silicone polymer so that hydrogen bonding, dipolar interaction and electrostatic interaction may occur within the color coating of the keratin fibers. The heteroatoms and heteroatom groups may include but are not limited to oxygen, nitrogen, sulfur, phosphorus, hydroxyl, carboxamido, sulfonamido, urethane, urea, acylurea and any combination thereof. The organic group C may be the same organic group as mentioned for A₁, A₂ and A₃ except that C will not have a heteroatom and/or heteroatom groups that confer functional polarity on C. Preferably, the organic groups A₁ though A₃ are linear, branched or cyclic aliphatic, heteroaliphatic, aromatic, heteroaromatic moieties or any combination thereof comprising 1 to 26 carbons (3 carbons minimum for branched and cyclic moieties) together with the foregoing heteroatoms and heteroatom groups and any combination thereof. Organic group C preferably is the same but without heteroatoms and heteratom groups. More preferably the organic groups A₁ though A₃ are aliphatic or heteroaliphatic moieties of 1 to 14 carbons with heteroatoms and heteroatom groups and any combination thereof (for branched or cyclic aliphatic and heteroaliphatic moieties, the minimum carbon number is 3). More preferably organic group C is the same but without heteroatoms and heteroatom groups. Especially more preferably, the organic groups A₁ though A₃ are linear alkyl moieties of 1 to 10 carbons or branched or cyclic alkyl moieties of 3 to 10 carbons with heteroatoms and heteroatom groups, and any combination thereof. Especially more preferably, organic group C is the same but without heteroatoms and heteroatom groups. The organic groups, the preferred organic groups and more preferred organic groups designated as A₁ through A₃ may have within the carbon chain, one or more ether groups, one or more thioether groups, one or more secondary or tertiary amino groups, one or more hydroxyl groups, one or more carboxamido groups, one or more sulfonamido groups, one or more urethane groups, one or more urea groups, one or more acylurea groups or any combination thereof.

As organic groups with heteroatoms and/or heteroatom groups, the organic groups A₁ though A₃ may incorporate one or more polar substituents selected from electron withdrawing, or electron donating groups with Hammett sigma para values between −1.0 and +1.5. Hammett sigma para values are discussed in Rompp Chemie Lexikon, Georg Thieme Verlag, Stuttgart, N.Y., 9th Edition, 1995 under “Hammett Gleichung”. The polar substituents can be non-ionic, zwitterionic, cationic or anionic and can include valence substituents completing the valence requirements of the polar substituents. The valence substituents include aliphatic and/or aromatic groups R₁, R₂, R₃, and R₄ as defined below. The polar substituents include S-linked groups such as but not limited to SO₂H, SO₃H, SR₁, SCN, S0₂R₁, S0₃R₁, SSR₁, SOR₁, SO₂NR₁R₂, SN R₁R₂, S(NR₁)R₂, S(0)(NR₁)R₂, SR₁(NR₁), SON R₁R₂. The polar substituents include N-linked groups such as but not limited to NR₁R₂, NR₁R₂R₃+, NC, NR₁OR₂, NR₁SR₂, NCO, NCS, NO2, N═NR₁, N═NOR₁, NR₁CN, N═C═NR₁, NR₁NR₂R₃, NR₁NR₂NR₃NR₄, NR₁N═NR₂. The polar substituents include carbon-linked groups and miscellaneous groups such as but not limited to COOH, COX, CON₃, CONR₁R₂, CONR₁COR₂, C(═NR₁)NR₁R₂, CHO, CHS, CN and NC wherein X is a halogen.

The R₁, R₂, R₃, and R₄ groups of the polar substituents may be each independently selected from hydrogen, straight, branched or mono- or polycyclic aliphatic, mono or polyunsaturated alkyl, aryl, heteroalkyl, heteroaliphatic or heteroolefinic moiety comprising 3 to 150 carbon atoms together with 0-50 heteroatoms, especially O, N, S, P. Preferably, R₁, R₂, R₃, and R₄ are each independently selected from hydrogen straight, branched or cyclic alkyl groups of 1 to 6 carbons (minimum of 3 for branched and cyclic), more preferably independently selected from hydrogen, methyl, ethyl, propyl or isopropyl, especially more preferably independently selected from hydrogen or methyl.

Reactive-Polymers from Preformed Polymers

The foregoing organic, silicone and organosilicone preformed polymers may be seen as precursors for the reactive polymers. Describing the reactive-polymer in this manner provides a realization of the breadth of the kinds of preformed polymers that can function as the reactive-polymer through incorporation of the first functional groups and second functional groups. When transformed into reactive polymers with complementary pairs of first and second functional groups, the organic and silicone polymers, oligomers and/or small molecules will have two kinds of substituents. The first kind may be but is not limited to chain and pendant groups that can hydrogen bond and/or dipolar interact but not enter into covalent reactions with complementarily reactive groups (hereinafter non-reactive groups). The second kind constitutes the first functional groups that are designed to react covalently with complementarily reactive groups (second functional groups).

Hence, the reactive-polymer can be conceptualized as a preformed polymer with non-reactive groups into which has been incorporated the first functional groups to form a reactive polymer first component and second functional groups to form a reactive polymer second component. The first and second components of the reactive polymers constitute reactive polymer pairs such that the first and second functional groups are pairs of complementary reactive groups. The reactive-polymer is constructed so that the first component contains first functional groups but not second functional groups. The reverse applies to the second component. The separation of complementary reactive pairs of first and second functional groups enables storage of the first and second components without special conditions.

The preformed organic and silicone polymers described above may be serve as templates for the reactive-polymers through incorporation of one or more monomeric units bearing first functional groups including but not limited to carboxylic acid groups, sulfonic acid groups, sulfinic acid groups, hydroxyl groups, mercapto groups, olefinoyloxy groups, vinyl groups, amine groups, epoxy groups, hydroxysilyl and/or alkoxysilyl groups and any combination thereof. Incorporation of appropriate monomeric units bearing first functional groups or protected first functional groups may be accomplished through ordinary polymerization techniques. These techniques are described in the scientific literature such as in J. March, “Advanced Organic Chemistry”, 4^{th} Ed. John Wiley & Sons, New York, 1992.

### Reactive Organic and Silicone Polymer Composition

The reactive-polymer composition comprises pairs of reactive-polymers having complementary reactive groups (first and second reactive functional groups) that covalently bond in situ to form the polymer product. The reactive polymer may also have but are not limited to non-reactive groups that may but not necessarily display such functions and hydrogen bonding and dipolar interaction. Embodiments of the polymer product may be chain extended, cross linked and/or a combination of both to form branched, network, star microstructure configurations and combinations thereof. The pairs of reactive-polymers comprise first and second components, each of which may be organic, silicone and/or organosilicone reactive-polymers and/or oligomers. The first component typically is a polymer with first functional groups and may include non-reactive in-chain and pendant groups that enable hydrogen, dipolar and ionic interactions. The second component is also known as an in situ linking material. The second component may be a small molecule, an oligomer or a polymer with second functional groups. Like the first component, the second component may also include non-reactive in-chain and/or pendant groups that enable hydrogen, dipolar and ionic interactions. The first and second functional groups are complementary reactive groups as described above.

Although it is not a limitation of the invention, it is believed that when the reactive-polymer has negligible water solubility or dispersibility, the melding of the three components to form a remanent coating may not be as efficient as can occur with better water solubility or dispersibility of the reactive-polymer. It is believed that increased melding can be accomplished by addition of substituents to the reactive-polymer that will increase the water solubility or dispersibility, preferably up to a limitation indicated by substantial water solubility. The range of solubility may be related to the characteristics of the reactive-polymer to form an opaque or cloudy dispersion in neutral water and the dispersion becomes less opaque or cloudy or becomes hazy when the pH is increased. Such substituents include but are not limited to acid groups, hydroxyl groups, ether groups, amide groups, ester groups, urethane groups, urea groups and functional groups that can exhibit hydrogen bonding.

### Organic Polymer First Component

Embodiments of the reactive-polymer as an organic polymer First Component may comprise a random distribution of the different monomer units along the polymer and/or oligomer backbone and/or branches or may be block copolymers which has blocks of single monomer units or may be a graft copolymer which has one monomer unit forming the polymer backbone and a different monomer unit forming polymeric side chains. The different constructions of polymer provide differing polymer to polymer binding properties and different macromolecular characteristics. The block copolymer can provide regions of hard and soft polymer characteristics. A block copolymer can display crystalline regions and amorphous regions that can enable development of water soluble and water resistant regions. Blocks of differing electronic and lipophilic character can impart an open repulsive character to the polymer so that tightly fit inter-structures are minimized. A graft polymer or segmented polymer is capable of intertwined conformation and compact molecular dimension so as to enable tightly fitted inter-structures.

Embodiments of the organic First Component may comprise a polyolefin, a polyvinyl, a polyester, a hydroxylated polyester, an acrylate functionalized polyester, a polycarbonate, a polyallyl alcohol, a ketone resin, a polyether, a polyimine, a polyurethane, a polyurea, a polyglycol, a polyamide, a polypeptide, poly (2-oxazoline) and its derivatives, a carbohydrate compound, a cellulose, a cellulose derivative such as a cellulose ester or a hydroxylated cellulose or a carboxyl cellulose or a hydroxyl cellulose ester or carboxylic acid, an alginate, a gum, a polysaccharide, an amino acid polymer, a gelatin, an oligopeptide, a polypeptide or a protein, a carbohydrate-amino acid such as a glycosylated peptide or a carbohydrate-purine/pyrimidine base such as a polynucleoside, a biopolymer, a (meth)acrylic copolymer, a crotonic copolymer, a polyurethane-polyglycol copolymer, a polycarbonate diol, a styrene-allyl alcohol copolymer, a polyol, a natural gum, polyvinyl acetate, polyvinylpyrrolidone, polynipam, a polymer based on one or more olefin monomers, a polymer based on ester units of diacids/diol monomers, a polymer based on ester units of hydroxy acid monomers, a polymer based on ether monomeric units, a polymer based on thioether monomeric units, a polymer based on polyol monomeric units, a polymer based on alkylene oxide monomeric units, a polymer based on of alkylene imine monomeric units, a polymer based on urethane monomeric units, a polymer based on urea monomeric units, a polymer based on amide units of diacid/diamine monomers, a polymer based on amide units of amino acid monomeric units or other polymer having repeating residues based on carbon or carbon in combination with other atoms such as oxygen and/or nitrogen, and any combination thereof. Especially preferred are polyolefins, polyvinyls, polyesters, polyurethanes and polyethers. More especially preferred are polyolefins, polyurethanes, polyesters and combinations thereof, typically also substituted by non-reactive groups as chain and/or pendant substituents.

The reactive-polymer may be linear and/or branched and/or star and may incorporate along the polymer backbone, as well as along the branches, pendant moieties such as esters, ethers, urethanes, ureas, oxycarbonyls, amides, aliphatic groups, aromatic groups, linear, branched or cyclic alkyl groups or similar non-reactive groups that are polar but are not complementarily reactive.

The first functional groups of the reactive polymer differ from the nonreactive groups. The first functional groups may be arranged as pendant groups, arranged as terminal groups or may be a combination thereof. The first functional groups cannot be in-chain groups because their function is covalent reaction with the second functional groups described below. As pendant groups, the first functional groups may be distributed along the reactive-polymer backbone, along polymer branches or any combination thereof. The first functional groups may be singly or multiply arranged at a single location of the polymer and in either arrangement may be distributed throughout the backbone and branches. The first functional groups may be polar and/or protic groups designed to react with the second functional groups. The first functional groups include but are not limited to carboxylic acid groups, hydroxyl groups, amine groups, mercapto groups (i.e., thiol, —SH), sulfo acid groups (HO₃S—), sulfino acid groups (HO₂S—), vinyloxycarbonyl, olefinoyloxy including (meth)acrylyloxy or crotonyloxy, alkynyl, Si—OH groups, Si—OR groups, Si—OAc groups, Si—O—N═CHR groups or Si—CH═CH₂ groups, or any combination thereof. The number of first functional groups per molecule of organic polymer is at least two and preferably is at least three and more preferably at least four and most preferably at least five. Not all reactive-polymer molecules may bear the same number of first functional groups.

The first functional groups as pendant and/or terminal groups may be covalently linked to the reactive polymer chain through any manner of linear and/or branched carbon connection arrangements or units. The connection units may covalently bear one or a multiple number of first functional groups. These carbon connection arrangements may be but are not limited to a carbon connection unit comprising a linear, branched or cyclic C1-C24 alkylenyl, oxyalkyenyl, alkylenyloxy or oxyalkylenyloxy unit, a C2-C24 alkanoyl or oxyalkanoyl unit, a C6-C24 aromatic or oxyaromatic unit, a C5-C24 heteroaromatic or oxyheteroaromatic unit having one or two heteroatoms selected from nitrogen, oxygen and sulfur, a (C_{z}—O—C_{z})ₙ polyether unit wherein z is an integer of 1 to 6 and n is an integer of 2 to 6, a (C_{y}—NH—C_{y})ₘ polyimino unit wherein y is an integer of 1 to 6 and m is an integer of 2 to 6. The recitation of “oxy” before or after an organic group means that the organic group such as alkylenyl is connected to the polymer chain through an oxygen. For example, an alkylenyl group is connected to the polymer chain by a carbon-carbon bond while an oxyalkylenyl group is connected to the polymer chain by a carbon-oxygen bond.

Representative embodiments of the organic polymer first component are set forth in WO/2019/211050 published Nov. 7, 2019, the disclosure of which is incorporated herein by reference.

### Silicone, Ormanosilicone First Components

The first functional groups may also be covalently linked to a silicone polymer or an organosilicone polymer or an organic polymer described above as a preformed organic, silicone or organosilicone polymer through linear and/or branched silicon connection units comprising a Si1-Si48 organosiloxane moiety (as R₂Si0₂ monomeric residues) having methyl as the organo group with silicon of the connection unit bonded to the first functional group through an alkylenyl group of one to three carbons or through an oxyalkylenyl group of one to three carbons or through an oxyalkylenyloxy group of one to three carbons and combinations thereof.

When the first functional group is Si—OH, Si—OR, Si—OAc, Si—O—N═CHR or Si—CH═CH₂ group wherein R is C1-C6 alkyl, this group is bonded to the reactive-polymer as an organic polymer through a moiety of Formula X: —(CH₂)ₙ—Oⱼ—Si(R¹)ₐ(R²)₃₋ₐ. This group may be bonded directly as a silyl moiety through a silyloxy group when the reactive-polymer is a polysilicone. The designator n for Formula X is an integer of 0 to 6, the designator j is zero or 1, R¹ is alkoxy of 1 to 3 carbons or OH or OAc or —O—N═CHR or —CH═CH₂, R² is alkyl of 1 to 3 carbons and a is an integer 1, 2 or 3. Formula X is incorporated into the organic polymer through the valence bond connection to a monomeric group compatible with the monomeric groups of the organic polymer. For example, if the organic polymer is a polyolefin, the open valence of the Formula X is bonded to a vinyl group or to an oxygen of an olefinoyloxy group such as (meth)acryloxy. The vinyl or olefin group is polymerized into the olefinic polymer chain as one of the olefinic monomer groups.

Representative embodiments of the silicone and organosilicone polymer first component are set forth in WO/2019/211050 published Nov. 7, 2019, the disclosure of which is incorporated herein by reference.

### Second Component

The second component of the reactive polymer pair, also described herein as in situ linking material, may comprise organic small molecules, organic oligomers, organic polymers, siloxanes, polysiloxanes, polyorganosiloxanes or polysilicones. More specifically, embodiments of the second compound may comprise a linear and/or branched organic or silicone core to which is bonded the second functional groups. The second functional group or groups comprise at least isocyanato, thioisocyanato, linear, branched or cyclic epoxyalkyl, malonic anhydrido, formyl, amino, hydroxyl, mercapto, furanyl, cyclopentadienyl, azide or olefinoyloxy such as (meth)acrylyloxy, crotonyloxy. The organic core may be an organic small molecule including a saturated aliphatic compound or an aromatic compound. The organic core may also be an organic oligomeric compound or an organic polymeric compound designated by the symbol Cpd. The silicone core may be a siloxane, a polysiloxane, a polyorganosiloxane or a polysilicone designated by the symbol Sicpd. The backbone and/or branches of the organic core and the silicone core are bonded to at least two pendant or terminal or pendant and terminal second functional groups. The second functional groups may be distributed throughout the core including the backbone and branches. The second functional groups may be singly or multiply arranged at a single location of the polymer and in either arrangement may be distributed throughout the backbone and branches.

The second functional group in coordination with the chemical and physical properties of the core of the second component provides in situ linkability with the first component having first functional groups including a hydroxyl, carboxyl, amine, mercapto, vinyl, olefinoyloxy, azide or Si—OH/SiOR/SiOAc/SiONCHR/Si-vinyl or any compatible combination thereof. These in situ linkable features enable the second component to be covalently, ionically, electrostatically, coordination-wise and/or entanglement-wise interactive with the first component.

Classes and embodiments of the organic, silicone and organosilicone polymer second component are set forth in WO/2019/211050 filed Nov. 7, 2019, the disclosure of which is incorporated herein by reference.

### Reactive Pairs of First and Second Functional Groups

The reactive-polymer composition presents first and second reactive polymer components with first and second functional groups disposed within the same class (autoreactive) and also presents first and second reactive polymer components with separated first and second functional groups (so as to facilitate bonding of first and second materials via complementary functional groups) that may be arranged so as to constitute reactive pairs. As discussed above, not all members of a functional group class are compatible with each other. For example, the presence of isocyanate and amine or hydroxyl on the same second component would lead to undesired self-reaction of the second component. Other differing members of a functional group class are compatible with each other, such as carboxyl, amine, hydroxyl and mercapto. Although these members of the first functional group have the potential for forming amide, ester and thioester groups, the activation energy needed to cause such as conversion is too high to enable reaction at typical and customary environmental conditions. A catalyst or activation agent is customary in such circumstances. For these reasons, selection of differing members of the first functional group to include within a first component reactive-polymer and selection of differing members of the second functional group to include within the second component reactive-polymer will account for the ability or lack of ability of the differing members to undergo undesired combination under ordinary conditions. The choice of first and second functional groups for the first and second components follows the principle of complimentary reactive pairs. These pairs are:
(i) isocyanate or thioisocyanate and hydroxyl, amine or mercapto or any combination of hydroxyl, amine and mercapto;
(ii) carboxyl and hydroxyl, amine or mercapto or any combination of hydroxyl, amine and mercapto in combination with catalyst;
(iii) epoxy and hydroxyl, amine or mercapto or any combination of hydroxyl, amine and mercapto;
(iv) cycloalkylepoxy and hydroxyl, amine or mercapto or any combination of hydroxyl, amine and mercapto;
(v) olefinoyloxy and hydroxyl, amine or mercapto or any combination of hydroxyl, amine and mercapto (the Michael addition);
(vi) melonic anhydride and hydroxyl, amine or mercapto or any combination of hydroxyl, amine and mercapto;
(vi) formyl (—CHO) and amine or mercaptan or any combination of amine and mercapto;
(vii) azido and alkynyl;
(ix) vinyl and mercapto or amine and any combination of amine and mercapto;
(x) mercapto and mercapto;
(xi) carbodiimide and carboxyl or amine (the polyacylurea formation);
(xii) oligo or polycyclic carbonate and amine (polyurethane formation by a non-isocyanate route)
(xii) any combination of Si—OH, Si—OR, Si—OAc, Si—O—N═CHR (the alkoxysilyl condensation);
(xiii) Si—H and Si—CH═CH₂.

Preferable reactive pairs of the preceding paragraph include the isocyanate/thioisocyanate and its counterparts, entry (i) above; epoxy and its counterparts, entries (iii and iv) above; olefinoyloxy and its counterparts, entry (v) above; vinyl and mercapto and its counterparts, entry (ix) above; mercapto and mercapto, entry (x) above; and silanol or alkoxysilane and its counterparts, entry (xi) above. More preferred reactive pairs include the isocyanate/thioisocyanate pairs, the epoxy pairs, the mercapto pairs and the silanol/alkoxysilane pairs. Especially more preferred reactive pairs include the isocyanate pairs, the silanol/alkoxysilane pairs and the epoxy pairs. Most preferred reactive pairs include the isocyanate pairs.

Exemplary further disclosure of the reactive pairs of first and second functional groups, preferences and examples are provided by the disclosure of PCT application WO/2019/211050 filed Nov. 7, 2019, the disclosure of which is incorporated herein by reference.

The highly preferred urethane functional group prepared from the preferred isocyanate reactive pair may alternatively and preferably be prepared from a small molecule, oligomer or polymer containing in-chain or pendant groups of 5 or 6 member cyclic carbonate rings. This first complementary reactive group is combined with an alkoxysilylalkylamine. The amine group opens the carbonate ring to form a urethane group and the displace hydroxyalkyl group from the carbonate ring reacts with the alkoxysilyl group of another alkoxysilylalkyl amine in a condensation reaction to form an alkoxysilylalkyl moiety bound to the free amine nitrogen. The cycle is repeated with additional cyclic carbonate and amine compounds to form a poly siloxy organic polymer having siloxy and urethane in chain groups. Alternatively these cyclic carbonates described can also react with an organic non silicone based polyamine e.g. a polyetheyleneimine to create repeating polymeric urea bonds within a polymer network.

### Michael Addition

A subcategory of the reactive-polymer comprising first and second components is the Michael Addition reactive-polymer pair. The Michael Addition first and second components may each comprise linear, branched, cyclic, cage, dendritic, star or fullerene-like reactive-polymer of organic, silicone or organosilicone oligomer and/or polymer construction with the first and second functional groups being complementary Michael Addition groups. The first and second compounds may also be grafted or block copolymers of organic and silicone segments. Preferred configurations of the first and second components are the linear and branched arrangements of the backbones, chains and frames of the small molecules, monomer group, oligomer and polymer. Especially preferred configurations of the first and second compounds are the linear arrangements. The organic and silicone compounds may be small molecules such as saturated organic groups or siloxane groups, aromatic organic groups, monomeric groups, oligomers or polymers. The first organic or silicone or organosilicone component carries as a first complementary functional group an α, β unsaturated acyl group such as for example olefinoyloxy group. The first component carries at least 2 functional groups which may be attached through optional connecting units to the organic or silicone chain, backbone, side chain or small molecule frame. The second organic or silicone or organosilicone component carries as the second complementary functional group an amine and/or a nitrogen with the amine being a pendant amine group and the nitrogen being a nitrogen as part of the frame, chain, backbone and/or side chain. The second compound carries at least 2 functional groups which may be attached through optional linking groups to the organic or silicone chain, backbone, side chain or small molecule frame and/or may be contained within the frame, chain, backbone and/or side chain.

These two complementary functional groups combine through a nucleophilic addition of the amine/nitrogen to the olefinic bond of the α, β unsaturated carboxyl group to form a nitrogen-carbon bond which is as the “Michael Addition.” While the nitrogen and/or amine group is the preferred nucleophile in this regard, thiol or mercaptan groups and carboxylate groups may also be employed for the same purpose. For these versions of nucleophiles, the second component may be modified to present sulfur nucleophiles or carboxylate nucleophiles instead of or in addition to nitrogen nucleophiles. Additionally, the first component with the pendant α, β unsaturated carboxyl ester group may also contain carboxylic acid or protected carboxylic acid groups. Upon neutralization with a base such as weak carbonate or hydroxide or upon deprotection to produce carboxylate anions, the carboxylate may function as a nucleophile and add to the pendant α, β unsaturated carboxyl ester groups. In this embodiment, the first component may be enabled to be self-reactive to provide cross linking in addition to or in replacement of the combination with the second component. In a similar fashion, a pendant thiol/mercaptan group which can function as a nucleophile as an —SH group can alternatively be treated with a base such as hydroxide to produce a thiol anion which will function as a stronger nucleophile if desired.

The linear, branched, dendritic, star, cage, cyclic or fullerene-like organic compound or linear, branched, cage, cyclic or dendritic silicone compound (preferably linear) comprises a small molecule, a monomeric group, an oligomer or polymer having at least two and at least preferably three complementary functional groups. The functional groups are complementary in that they react together in situ with minimal activation energy to provide cross linked first and second compounds. When the pre-treatment composition as an amine polymer is also present (hereinafter third component), it may also enter into the nucleophilic addition to provide additional cross-linking and also interacts through electrostatic and lipophilic activity. The complementary functional groups covalently bond together in situ to meld together the first, second and third components to form a remanent coating. The coating also includes ionic, electrostatic, entanglement and/or coordination interactions between the molecules of these components and also between the components and the surfaces of the substrate material, preferably keratin material.

The reactive functional groups are distributed throughout the frame of the small molecules and along and within the backbones and branch chains of the monomeric group, the oligomer and the polymer of the first and second compounds. The reactive functional groups comprise olefinoyloxy groups and amine/nitrogen groups. The complementary functional groups can react together under ordinary environmental conditions and/or can react together with the help of a fourth component such as catalyst or substance that will lower the reaction activation energy needed for the reactive bonding of the functional group pair. Treatment with heat such as by a hot air blower, hot iron or other heat producing device may facilitate the reaction of the first and second functional groups.

### The First and Second Functional Groups, Formulas I and II

With appropriate selection of the functional groups on the compounds, cross linking, net and/or star formation of the coating can be achieved. The complementary reactive pair functional groups can be designated as Formula I and Formula II. These Formulas include double valence connector units R¹ and R^{4a} that respectively join the olefinoyl/olefinoyloxy and amine/nitrogen groups to the remainder of the first and second compounds. These reactive functional units for the first and second compounds respectively have Formulas I and II:

—R¹—O—CO—CR²═CHR³ Formula I:

—R^{4a}—N(R⁴)₂ Formula II:

As mentioned above, the first and second compounds respectively have at least two and preferably at least three functional units per molecule.

For Formula I, R¹ is a double valence linear or branched alkyl group of 1 to 12 carbons optionally with one or more heteroatoms in or on the alkyl chain, a cycloalkyl-alkyl or cycloalkyl group of 3 to 12 carbons optionally with one or more heteroatoms in the cycloalkyl ring and/or alkyl chain, an arylalkyl group of 6 to 20 carbons optionally with one or more heteroatoms in the alkyl chain, an aryl group of 6 to 20 carbons, or a heteroaryl group of 6 to 20 carbons containing one or more heteroatoms. The heteroatoms may be oxygen, nitrogen and/or sulfur. R¹ may also be substituted by hydroxyl, especially and preferably in a position to form a pseudo seven member hydrogen bonding ring with the carbonyl of the olefinoyloxy group.

R² and R³ may be hydrogen or an alkyl group of 1 to 4 carbons. Preferably when one or both of these groups is alkyl, the alkyl is methyl. When R² is hydrogen and R³ is hydrogen, the olefinoyloxy group is an acryloxy group. When R² is methyl and R³ is hydrogen, the olefinoyloxy group is a methacryloxy group. When R² is hydrogen and R³ is methyl, the olefinoyloxy group is a crotonyloxy group. When R² is methyl and R³ is methyl, the olefinoyloxy group is a 2-methyl-2-butenoic acid or tiglic acid.

The group —R^{4a} may comprise a single or double valence group and typically is a double valence group. As a double valence group, R^{4a} binds Formula II to the remainder of the second component such that Formula II comprises a pendant or in-chain amine group. In this context, the group —R^{4a} may comprise a linear or branched alkyl radical of 1 to 12 carbons optionally substituted by a primary amine group and optionally containing with one or more heteroatoms in the linear or branched alkyl chain, a cycloalkyl-alkyl or cycloalkyl of 3 to 12 carbons optionally substituted by a primary amine group and optionally containing one or more heteroatoms in the cycloalkyl ring and/or alkyl chain, an arylalkyl of 6 to 20 carbons optionally substituted by a primary amine group and optionally containing one or more heteroatoms in the alkyl chain, an aryl group of 6 to 20 carbons optionally substituted by a primary amine group, or a heteroaryl group of 6 to 20 carbons containing one or more heteroatoms and optionally substituted by a primary amine group.

In its single valence embodiment, —R^{4a} functions as a nitrogen substituent such as a methyl or an ethylamine group bonded to the nitrogen. The free valence (−) in these instances for —R^{4a} can bind it to nitrogen and to the remainder of the second compound as a double free valance or it can bind R^{4a} only to nitrogen as a single free valence. The one or more heteroatoms independently of R^{4a} may be nitrogen, oxygen, sulfur or a combination thereof.

Each R⁴ independently is hydrogen, alkyl of 1 to 6 carbons, —R^{4a} or a free valence bond provided that at least one R⁴ is hydrogen. When one of the R⁴ groups is a free valence, the nitrogen is part of a small molecule frame or is a nitrogen in the backbone, chain or side chain of a monomeric group, an oligomer or a polymer. Combined with —R^{4a} as a single valence group described above, examples of R⁴ provide Formula II as linear and branched polyethylene imine, polypropylene imine as well as di, tri, tetra, penta ethylenylamine. Another example is triethyleneimine which has the formula CH₃CH₂—NH—CH₂CH₂—NH—CH₂—CH₂—NH₂. As Formula II, this example has —NH— in the chain with ethyl being the selection of R^{4a}, ethylenyl with two free valences being a first selection of R⁴ and ethylenyl amine being a second selection of R⁴.

A more detailed disclosure of Formulas I and II and exemplary embodiments of the Michael Addition first and second components is provided by the disclosure of PCT application WO2020/008073 published Jan. 9, 2020, the full disclosure of which is incorporated herein by reference.

### Carbodiimide (CDI) Reactive Polymer Pairs

Another subcategory of the reactive polymer comprising first and second components is directed to carbodiimide linking with acid, amine or thiol groups (hereinafter the CDI reactive polymer pair). The first component of the CDI reactive polymer pair comprises an CDI link polymer. The CDI link polymer comprises homopolymers, copolymers, terpolymers, multiple monomeric unit polymer embodiments and/or small molecules having at least two pendant CDI link groups. The CDI link groups may comprise acid groups including carboxylic acid groups, sulfonic acid groups and phosphoric acid groups. The CDI link groups may also comprise amine groups or thiol groups. The amine and/or thiol groups may be present alone in the CDI link compound or may be present in combination with the acid groups; however, the preferable arrangement is a CDI link polymer with amine or thiol groups alone when a compound with amine or thiol groups is to be employed. Overall, carboxylic acid groups are preferred for all versions of the CDI link polymer including organic polymers, silicone polymers, organosilicone polymers and small molecules. Most preferably the CDI link polymer is a carboxylic acid polymer with optional reactive termini. As an organic, silicone or organosilicone polymer, the CDI link polymer and especially the acid bearing polymer may be configured to have a linear, branched, cyclic, dendritic, star, graft, block, or fullerene structural arrangement or any combination thereof. The polymer may be an organic polymer, a silicone polymer or an organosilicone polymer of a block configuration. Further a polycarbodiimide when added in excess to the reactive groups and when water is present will form a polyurea which may further improve the toughness of the film and adhesion to hair.

### CDI Link Organic Polymers

Embodiments of the CDI link polymer comprise embodiments of one or more organic polymers bearing two or more CDI groups, preferably acid groups and more preferably carboxylic acid groups

The CDI groups may be arranged on the organic polymer as pendant groups, arranged as terminal groups or may be a combination thereof. The CDI groups may be distributed along the organic polymer backbone, along polymer branches or any combination thereof. The CDI groups may be singly or multiply arranged at a single location of the polymer and in either arrangement may be distributed throughout the backbone and branches. The number of CDI groups per molecule of organic polymer and especially the number of carboxylic acid groups per molecule of organic polymer is least two and preferably is at least three and more preferably at least four and most preferably at least five. Not all organic polymer molecules may bear the same number of CDI groups. For the foregoing arrangements and preferences, the preferred CDI group is carboxylic acid.

Embodiments of the CDI link organic polymer are set forth in detail in PCT application wo 2020/114647 filed Oct. 1, 2019, the disclosure of which is incorporated herein by reference.

### CDI Link Small Molecules

The CDI link small molecules include aliphatic and/or aromatic and/or heteroaromatic polycarboxylic acids, polysulfonic acids, polyphosphoric acids, polyamines and/or polythiols having 4 to 100 carbon atoms with optional nitrogen, oxygen and/or sulfur atoms and at least two carboxylic acid groups sulfonic acid groups, phosphoric acid groups, amine groups or thiol groups. Preferably, these small molecules include alkane polycarboxylic acids of 2 to 30 carbons, alkene polycarboxylic acids of 4 to 30 carbons and at least one unsaturation, aromatic polycarboxylic acids and heteroaromatic polycarboxylic acids. Exemplary embodiments of CDI link small molecules are set for the in detail in PCT application serial no PCT/EP2019/076647 filed Oct. 1, 2019, the disclosure of which is incorporated herein by reference.

### CDI Link Silicone Polymers

Embodiments of the CDI link compound concerning the CDI link silicone is comprised of a MDTQ silicone polymer having at least two pendant organic groups bearing carboxylic acid groups, sulfonic acid groups, phosphoric acid groups, amine groups or thiol groups, preferably carboxylic acid groups. The silicone polymer includes a non-functional (non-acid bearing) portion and an acid bearing portion. An acid bearing silicone polymer including the non-functional portion and the carboxylic acid portion is one having carboxylic acid functional groups (hereinafter an acid bearing silicone polymer). For the acid bearing silicone polymer, the molar ratio of M type and/or D type siloxane monomeric units providing the at least two pendant organic carboxylic acid groups (hereinafter SiA units) to M type and/D type siloxane monomeric units having silicon bonded to a substituent (R) selected from groups such as but not limited to alkyl (C1 to C6), amidoalkyl (C1 to C6), alkylamide (C1 to C6), sulfonamidoalkyl (C1 to C6) and alkylsulfonamide (C1 to C6) and any combination thereof.

A detailed disclosure and exemplary embodiments of CDI link silicone polymers are set for the in detail in PCT application serial no PCT/EP2019/076647 filed Oct. 1, 2019, the disclosure of which is incorporated herein by reference.

### Optional Reactive Termini for the First Component

The organic and/or silicone polymer of the first component may optionally have reactive termini designed to self-combine or to combine with the fourth component. The reactive termini option for the first component includes groups other than the CDI link groups that combine with carbodiimide. In this optional form, the reactive termini for the first component are capable of self-reacting to chain extend the organic and/or silicone polymer molecules or may react with a co-reactant (the fourth component) to couple the organic and/or silicone polymer molecules together, thus also chain extending these polymer molecules. The reactive termini include hydroxysilyl groups, alkoxysilyl groups, silyl hydride and silyl-vinyl groups, Michael addition groups, epoxy and nucleophilic groups such as amines, thiols and hydroxyls, and Schiff base formation groups such as carbonyl and amine groups. A detailed disclosure and exemplary embodiments of optional reactive termini for the first CDI component are set forth in detail in PCT application serial no WO 2020/114647 filed Oct. 1, 2019, the disclosure of which is incorporated herein by reference.

### Second Component: Polycarbodiimide

The second component of the CDI link polymer pair comprises a polycarbodiimide. The polycarbodiimide has at least two carbodiimide groups, preferable from about two to about one thousand carbodiimide groups per molecule, more preferably from about two to about five hundred carbodiimide groups per molecule and most preferably up to about 200 carbodiimide groups per molecule. The carbodiimide groups of the polymer may be arranged as a linear chain or as pendant groups attached to an organic polymer chain. The polycarbodiimide is capable of reaction with all forms of the CDI groups mentioned above and preferably, the polycarbodiimide may be combined with the acid bearing compound (acid bearing organic or silicone or organosilicone polymer or small molecule) having carboxylic acid groups. In the subsequent passages, the preferred carboxylic acid-polycarbodiimide combination is discussed with the understanding that the other CDI link groups may also function in this capacity. With this preference in mind, the combination of the acid bearing compound of the first component and the polycarbodiimide provides a polymeric network connected by N-acyl urea bonds.

More specifically, the polycarbodiimide embodiments of the second component have Formula V. These embodiments may be maintained in neet condition or in compatible, inert solvent as a separate second component of the multicomponent composition until the multicomponent composition is to be applied to keratin material.

Z-(L-N═C═N—)ₚ—Z Formula V

In Formula V, Z is a terminal group of the polycarbodiimide and may be reactive or inert and preferably is reactive. L is an organic linker group derived from the formation of Formula V by catalytic conversion of diisocyanates of the formula OCN-L-NCO. The designator p is an integer of at least 2 so that the polycarbodiimide of Formula V contains at least 2 carbodiimide groups. The organic linker group L may be an aromatic, alkaromatic or saturated linear, branched or cyclic aliphatic divalent radical of 2 to 30 carbons optionally with one or more non-pendant heteroatoms including nitrogen, oxygen, sulfur or any combination thereof in the aliphatic chain or the aromatic group, such as but not limited to groups such as polyurethane or polyether as in polyethylene glycol. In its reactive form Z may react with itself to chain extend polycarbodiimide molecules or Z may react with a co-reactant (the fourth component) to couple polycarbodiimide molecules together, thus also chain extending the polycarbodiimide molecules.

Embodiments of Z include radicals of the formula Y—X wherein Y is an organic moiety and X is a reactive terminus or an inert terminus. As a reactive terminus, X may be configured as a reactive silyl group or as a reactive organic group. As an inert terminus, X may be configured as a linear, branched or cyclic saturated aliphatic group of 1 to 50 carbons or an aromatic group of 6 to 18 carbons, the aliphatic and aromatic groups optionally including from one to 10 heteroatoms selected from nitrogen, oxygen, sulfur or any combination thereof and the aliphatic or aromatic group may be partially or fully fluorinated.

A detailed description and exemplary embodiments of Y-X are set forth in detail in PCT application serial no PCT/EP2019/076647 filed Oct. 1, 2019, the disclosure of which is incorporated herein by reference. A detailed description, preferred embodiments and tablular characterizations of polycarbodiimides as the second component are set forth in detail in PCT application WO2020/114647 filed Oct. 1, 2019, the disclosure of which is incorporated herein by reference.

### Pre-Treatment Composition—the Amine Polymer

The significant remanence, wear-fastness and resistance to environmental attack of the color coating on keratin fibers according to aspects of the invention may be developed through interaction between any one or more of the color compositions and the pre-treatment composition. The interaction is the complexation and cooperation of interlayer hydrogen bonding, dipolar interaction, molecular intertwining and for the reactive polymer compositions, the covalent crosslinking between and among the amine polymer layer laid down on the keratin fibers and the overlaid polymeric films of one or more of the preformed polymer and the reactive-polymers. This aspect according to the invention is believed to produce at least in large part qualities and characteristics of the color coating on the keratin fibers. For this reason, incorporation of the pre-treatment composition into the dressing of keratin fibers according to the invention is a key and preferred aspect of the invention.

The embodiments of the pre-treatment composition combine with embodiments of the preformed polymer composition and/or the embodiments of the combination of the first and second components of the reactive-polymer composition to meld together (e.g., blend, combine, unite together as one) these components into a colored coating on keratin fibers that displays significant remanence. Embodiments of the substantive feature of the pre-treatment composition are a polymer with incorporation of amine groups into and onto an organic or silicone core or chain. Embodiments of the amine polymer as an organic core with amine groups may be one or more amine polymer(s). The amine polymer(s) may comprise one or more amino functional group(s) per polymer chain, wherein the amino functional group(s) are selected from the group consisting of primary, secondary and/or tertiary amino functional groups and mixtures thereof, preferably from the group consisting of secondary and tertiary amino functional groups and mixtures thereof.

Embodiments of the amine polymer may be selected from the group consisting of polyethyleneimine, polyallylamine hydrochloride, polydiallyldimethylammonium chloride, polyvinylamine, aminopolysaccharide, aminosilicones, copolymers thereof and mixtures thereof. The polymer(s) may preferably be selected from the group consisting of polyethyleneimine, aminosilicone, polydiallyldimethylammonium chloride, copolymers thereof and mixtures thereof. Additional examples include triethoxysilylamine, 1,1,1-triethoxy, 2,2-dimethyl-2-aminodisiloxane and aminoalkylmonoethoxydimethylsilane, aminoalkyldiethoxymethylsilane and aminoalkyltriethoxysilane with 1 to 6 carbons in the alkyl group, preferably 2 or 3 carbons.

A detailed description and exemplary embodiments of the amine polymer component of the pre-treatment composition are set forth in detail in PCT application WO 2020.114647 filed Oct. 1, 2019, the disclosure of which is incorporated herein by reference.

### Medium

The medium of the multicomponent composition embodiments of the invention may be an organic compound or silicone compound that is capable of being intimately mixed or preferably forming a solution with up to 20 percent by weight water. The preferred medium comprises embodiments of alcoholic solvents such as an alkyl alcohol of 1 to 6 carbons with no intentionally added water or with intentionally added water up to about 10 weight percent, preferably up to about 5 weight percent, more preferably up to about 2 or 3 weight percent relative to the total weight of the medium. It is recognized that alcohol solvents absorb water from the atmosphere so that an alcoholic solvent with no intentionally added water may contain a slight amount of water. Alternatively, the medium may be a volatile silicone liquid preferably with no water. Exemplary volatile silicones may be represented by cyclomethicone, cyclopentasiloxane, cyclohexasiloxane, trisiloxane, dimethicone, octamethyltrisiloxane, decamethyltetrasiloxane, dodecamethylpentasiloxane and similar siloxanes of up to 40 or 50 siloxy groups. The volatile silicone liquid may be combined with liquid hydrocarbons and/or non-hydroxylic, polar organic solvents, such as methyl ethyl ketone and similar solvents.

Alternatively, for the preformed composition, the pretreatment composition and certain of the reactive polymer compositions, the medium may be an aqueous medium optionally containing a minor concentration of miscible organic solvent or may be a two phase aqueous medium with water as the continuous phase and a water immiscible silicone or liquid organic compound as the discontinuous phase. The continuous phase may carry water soluble constituents while the discontinuous phase may carry constituents such as one of the first and second components of the reactive polymer composition that would react with water. The discontinuous, non-aqueous phase will tend to isolate such compounds from degradation by water. Preferably, in situations when water is to be part of a medium but one or more of the color composition constituents are sensitive to water, the color compositions are maintained in a non-aqueous environment until they are ready for dressing on keratin fibers. At this stage, a two phase medium may be prepared as appropriate.

The polarity and protonic character of the medium are important for control of the several reactions that occur when the components of the multicomponent composition are combined. These reactions include the carboxylic acid-carbodiimide reaction and the reactive termini reaction such as the alkoxysilyl condensation to form silyloxysilyl linkages. Preferably, the medium for the first and second components is an alkyl alcohol with no intentionally added water. Preferably the medium for the third component is water so that when the three components are applied to the treatable material to provide an inchoate coating, the alcoholic medium combined with water is present.

As shown by the experimental examples, it has been discovered that the identity of the medium plays a role in obtaining a substantially complete, uniform, appropriately sized coating of the applied multicomponent composition on the treatable material, especially keratin material and specifically hair. The examples show that when the medium for application is water, the multicomponent composition reaction rate is increased to an extent that development of a substantially uniform, appropriately sized coating is difficult to obtain. The examples also show that when the medium for application is a non-polar, aprotic, lipophilic solvent such as dodecane, the multicomponent composition reaction rate is so slow that development of the combined product occurs over a period of multiple hours if not days. The experimental examples provide an understanding that a balance of hydrophilic and lipophilic character and a small degree of protonic character provide an appropriate reaction rate. This balance is achieved through use of a linear monoalcohol of 1 to 6 carbons, preferably methanol, ethanol or propanol, more preferably ethanol as the medium. These alcohol solvents are not naturally anhydrous in that they absorb water from the atmosphere. Consequently, they may serve as the multicomponent medium without intentional addition of water. Nevertheless, a minor amount of water up to a maximum of about 10% preferably up to about 5%, more preferably up to about 2% by weight relative to the total weight of the medium, may be added to manage the hydrophilic/lipophilic character and ionic/protonic balance of the medium. The water addition is desirable when the polycarbodiimide and optionally the first component have reactive termini that are self-reacting, such as alkoxy silyl and/or hydroxysilyl groups.

According to at least one embodiment of the present disclosure, the organic solvent is chosen from ethanol, isopropanol, butanol or pentanol.

The medium with or without one or more volatile organic solvent may be present in the composition according to the present disclosure in an amount ranging from about 0.1% to about 99% by weight, such as from about 1% to about 98% by weight, for example ranging from 50% to 95% by weight relative to the total weight of the composition.

### Viscosity, Composition Concentrations

The viscosity of the composition functions to hold the composition with pigment microparticles in place on the treatable material while the in situ linked coating is formed. The viscosity substantially avoids free translational flow of the composition. Free translation flow would cause the composition to rapidly run and drip off the surfaces of the hair strands. Nevertheless, the viscosity is not so high that it will not undergo self-leveling to substantially uniformly coat the treatable material. Appropriate viscosity of the composition is the result of the interaction of the organic polymer, the in situ material, the base compound, their concentrations, the pigment microparticles, and as appropriate, an optional viscosity control agent, an optional suspending agent and an optional thickening agent. Generally, the viscosity of the composition may range from about 0.001 to about 2000 Pa s. Viscosity measurements are carried out on a controlled stress rheometer e.g. Using an AR2000 type manufactured by TA Instruments, or equivalent instrument. A 6 cm flat acrylic cross hatched parallel plate geometry (TA item 518600.901) and a stainless steel cross hatched base plate (TA item 570011.001) are used. The rheometer is prepared for flow measurements as per standard manufacturer procedure. The parallel plate geometry gap is set to 1000 microns. The flow procedure is programmed to the rheometer with the following conditions: continuous stress ramp 0.1-300 Pa over 2 minutes at 25° C., including 250 measurement points in linear mode. The product is loaded into the geometry as per standard procedure and the measurement commences at 5 min after the mixture preparation. Shear stress value at 10 sec⁻¹ shear rate is obtained from the shear stress vs. shear rate curve, and the corresponding viscosity is calculated by dividing the obtained shear stress by 10.

The concentration of the CDI link compound and preferably the acid bearing compound in the composition may range from about 0.1% to about 90%, preferably about 1% to about 40%, more preferably about 2% to about 30%, most preferably about 2% to about 15% of CDI link compound by weight relative to the total weight of the composition. Specific concentrations of acid bearing compound include about 0.1%, about 0.5%, about 1%, about 2%, about 4%, about 6%, about 8%, about 10%, about 12%, about 14%, about 16%, about 18%, about 20%, about 22% about 24%, about 26%, about 28% or about 30% by weight relative to the total weight of the composition. The concentration of the in situ linking material (the polycarbodiimide) may range from about 0.1% to about 50% and is approximately about half of the concentration by weight of the acid bearing compound. Preferably the preferred ranges and specific concentrations of the in situ linking material are approximately half of those for the acid bearing compound. These concentration ranges preferably apply when the acid bearing compound is other than an acid bearing small molecule. The concentration range of the third component is given above in the third component section. The concentration range of the fourth compound, when present, will be determined by the concentration of the in situ linking material and may be approximately from about half of, to about the same as the molar concentration of the in situ linking material. The determination of the concentration for embodiments of the Acid bearing compound and in situ linking material and third component will depend in part upon the resulting viscosity, the saturation point of the acid bearing compound and in situ linking material and base compound in the medium. As discussed above, the viscosity is managed so that the composition will not run off the surfaces of strands of hair yet will level and flow to substantially coat those surfaces. Development of appropriate viscosity in part by management of the concentration of the acid bearing compound and in situ linking material can be experimentally determined by routine methods such as formulation of several samples of differing concentrations of polymer in the composition, coating those samples on a hair tress and observing the flow, spread and leveling of the composition on the hair strands.

The multicomponent composition can be applied simultaneously, sequentially or in pre-combined form to a treatable material such as a hair tress using the coloring procedure described herein afterwards. The top of the hair strand, where it is glued together is clamped in a stand such that the hair is aligned vertically downwards. After a 5 minute dwell time it is observed if any and how much product has dripped from the hair tress. The results obtained from the several samples can be plotted against flow time and leveling time to determine an appropriate concentration or range of concentrations of the organic polymer in the composition. A preferred concentration of the combination of CDI link compound and in situ linking material in the composition ranges from about 1% to about 60%, more preferably about 2% to about 40% and most preferably about 3% to about 30% by weight relative to the total weight of the composition. The weight amounts of CDI link compound and in situ linking material may be determined by calculation of amounts needed to substantially or essentially, or completely react the carboxylic acid groups with the carbodiimide groups, also taking into account the optional interaction between the carboxylic acid groups and the amine groups of the third component. The calculation of amounts will depend upon the molecular weights or weight average molecular weights of the CDI link compound, the in situ linking material and the third component and their relative numbers of functional/reactive groups.

The extent of in situ linking between the first, second and third functional groups may be controlled by manipulation of ratios, amounts present and concentrations as well as by physical means as described above so that the mechanical and chemical properties of the coating as described herein are preserved. In connection with hair, these properties include ability to adhere to hair strands, ability to maintain flexibility and free flowing character of the hair, ability to provide remanence, avoidance of stickiness and avoidance of clumping.

The glass transition temperatures of the organic polymer and the in situ linking material and the base compound in part contribute to the flexibility, strength, hardness and similar qualities of the coating on the treatable material surfaces. The glass transition temperature of these embodiments are given with each section described above. This glass transition temperature or T_{g} determines the solid-solid transition of the polymer from a hard glassy material to a soft rubbery material. If the T_{g} of the polymer is too high, the coating on the treatable material will be stiff and inflexible. This is an undesirable result. The coating should be soft, flexible and unnoticeable to touch and sight yet should not flake, break-up or otherwise release from the keratin fiber, and especially from human hair, when stroked by a hand or brushed with a brush. The Tg of a polymer can be measured using ASTM D7426-08 (2008).

### Plasticizer

If the glass transition temperature of the multicomponent composition and or the substantive ingredients of the first, second and/or third components are too high for the desired use yet the other properties of the polymer are appropriate, such as but not limited to color and remanence, one or more plasticizers can be combined with the multicomponent composition embodiments so as to lower the T_{g} of the organic polymer and provide the appropriate feel and visual properties to the coating. The plasticizer can be incorporated directly in the coloring composition or can be applied to the hair before or after the coloring composition. The plasticizer can be chosen from the plasticizers usually used in the field of application. Appropriate selection includes choice of a plasticizer that does not interfere with or compete with the carbodiimide-CDI link group reaction or with the reactive termini combination.

The plasticizer or plasticizers can have a molecular mass of less than or equal to 5,000 g/mol, such as less than or equal to 2,000 g/mol, for example less than or equal to 1,000 g/mol, such as less than or equal to 900 g/mol. In at least one embodiment, the plasticizer, for example, has a molecular mass of greater than or equal to 40 g/mol.

Thus, the multicomponent composition can also comprise at least one plasticizer. For example, non-limiting mention can be made, alone or as a mixture, of common plasticizers such as: glycols and derivatives thereof, silicones, silicone polyethers, polyesterpolyols; adipic acid esters (such as diisodecyladipate), trimellitic acid esters, sebacic acid esters, azalaeic acid esters; nonlimiting examples of glycol derivatives are diethylene glycol ethyl ether, diethylene glycol methyl ether, diethylene glycol butyl ether or diethylene glycol hexyl ether, ethylene glycol ethyl ether, ethylene glycol butyl ether, or ethylene glycol hexyl ether; polyethylene glycols, polypropylene glycols, polyethylene glycol-polypropylene glycol copolymers, and mixtures thereof, such as high molecular weight polypropylene glycols, for example having a molecular mass ranging from 500 to 15,000, for instance glycol esters; propylene glycol derivatives such as propylene glycol phenyl ether, propylene glycol diacetate, dipropylene glycol ethyl ether, tripropylene glycol methyl ether, diethylene glycol methyl ether, and dipropylene glycol butyl ether. Such compounds are sold by Dow Chemical under the names DOWANOL PPH and DOWANOL DPnB; acid esters, for example esters of carboxylic acids, such as triacids, citrates, phthalates, adipates, carbonates, tartrates, phosphates, and sebacates; esters derived from the reaction of a monocarboxylic acid of formula R₁₁COOH with a diol of formula HOR₁₂OH in which R₁₁ and R₁₂, which can be identical or different, are chosen from a linear, branched or cyclic, saturated, or unsaturated hydrocarbon-based chain containing, for example, from 3 to 15 carbon atoms for example the monoesters resulting from the reaction of isobutyric acid and octanediol such as 2,2,4-trimethyl-1,3-pentanediol, such as the product sold under the reference TEXANOL ESTER ALCOHOL by the company Eastman Chemical; oxyethylenated derivatives, such as oxyethylenated oils, such as plant oils, such as castor oil; mixtures thereof.

Among the esters of tricarboxylic acids mention can be made of the esters of triacids wherein the triacid corresponds to formula

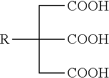

wherein R is a group —H, —OH or —OCOR′ wherein R′ is an alkyl group containing from 1 to 6 carbon atoms. For example, R can be a group —OCOCH₃. The esterifying alcohol for such tricarboxylic acids may be those described above for the monocarboxylic acid esters.

The plasticizer can be present in the composition of the present disclosure in an amount from about 0.01% to 20%.

### Pigment

The color composition embodiments of the present invention make it possible to obtain colored and remnant coatings, without substantially altering the keratin fibers. As used herein, the term “pigment” generally refers to any particle colorant having or containing pigment material that gives hair fibers color including black and white, such as titanium dioxide that give only white to hair fibers. The pigments are substantially water-insoluble. The pigments, to distinguish from dyes presented in molecular from, are also referred to as pigment microparticles or pigment particles. The terms pigment microparticles and pigment particles are synonymous and are used herein interchangeably. The pigments can be organic, inorganic, or a combination of both. The pigments may be in pure form or coated, for example with a polymer or a dispersant.

Selections, multiple kinds and varying forms of the pigment microparticles as described in the following passages can be incorporated in any of the first, second and third components of the multicomponent composition, or can be incorporated in any two of these components or in all three. Preferably, pigment microparticles can be incorporated in either or both of the first and second components. More preferably, pigment particles can be incorporated in the first component.

The at least one pigment that can be used can be chosen from the organic and/or mineral pigments known in the art, such as those described in Kirk-Othmer's Encyclopedia of Chemical Technology and in Ullmann's Encyclopedia of Industrial Chemistry. The pigments comprised in the microparticles comprising at least one pigment will not substantially diffuse or dissolve into keratin fibers. Instead, the pigment comprised in the microparticles comprising at least one pigment will substantially remain separate from but attached to the keratin fibers.

The at least one pigment can be in the form of powder or of pigmentary paste. It can be coated or uncoated. The at least one pigment can be chosen, for example, from mineral pigments, organic pigments, elemental metal and their oxides, and other metal modifications, lakes, pigments with special effects such as nacres or glitter flakes, and mixtures thereof.

### Pigment Shape

The pigment microparticles can have any suitable shape, including substantially spherical. But the pigment microparticles can also be oval, elliptical, tubular, irregular, etc., or even combinations of various shapes. In addition, the pigment microparticles can have two dimensions, length and width/diameter, of similar magnitude. In addition, the pigment microparticles can be micro platelets, i.e. having a thickness that is substantially smaller than the planar dimension. For example, five, ten or even 20 times smaller in thickness than in the planer dimension. In one embodiment with any of the reactive components of the instant invention, the pigments may be surface treated, surface coated or encapsulated.

In a particular aspect, the pigment microparticles can have a shape approximating that of a sphere, in which case the microparticles are referred to as being microspheres. Pigment microparticles which can be described as microspheres are understood as particles having an aspect ratio, defined as a function of the largest diameter, or largest dimension, dmax and the smallest diameter, or smallest dimension, dmin, which can be orthogonal to each other: AR=dmax/dmin which is from about 1:1 to 10:1, preferably from 1:1 to 5:1, more preferably from 1:1 to 4:1, such as from 1:1 to 3:1. More particularly, the expression “spherical-type” means that the pigment microparticles have a shape approximating that of a sphere. In other words, the pigment microparticles can be nearly orbicular in shape and can have a cross-sectional geometry that is essentially circular. Although not excluded, this does not necessarily mean that the pigment microparticles have the shape of a perfect sphere or ball. More likely, the shape of the pigment microparticles can exhibit a certain deviation from a sphere as long as the skilled person considers the shape as being similar to a sphere or as an approximation of a sphere.

In addition, the pigment microparticles can have a rather two-dimensional shape, with the smallest dimension substantially smaller than the two other dimensions, in which case the microparticles are referred to as being 2-dimensional microparticles. For example, the thickness of the microparticles can be significantly less than their length and width. The length and width can be of similar magnitude. Examples includes pigment microparticles having a shape of platelets, i.e. with a thickness that is substantially smaller than the planar dimension. For example, the aspect ratio AR=dmax/dmin, as defined above, of microparticles having a substantially two-dimensional shape, can be from about 10:1 to about 1000:1, preferably from about 10:1 to about 800:1, preferably from about 20:1 to about 800:1, preferably from about 10:1 to about 600:1, preferably from about 20:1 to about 600:1. Typically, the 2D-microparticles have a largest and a smallest dimension in their planer dimension, which both are significantly larger than the smallest dimension of the 2D-microparticles extending perpendicular to the planer dimension.

According to an embodiment, the pigments can include pigment microparticles of different shape. For example, microparticles of different size can be used to provide different reflecting and absorbing properties. Microparticles having different shape can also be formed of different pigment material. Furthermore, microparticles having different shape can also formed of different pigment material to provide different color.

### Pigment Size

The pigments can be present in the composition in undissolved form. Depending on the shape, the pigments can have a D50[vol] particle diameter of from 0.001 micron to 1 micron.

For example, pigments that can be described as being microspheres can have a D50[vol] particle diameter of from 0.01 micron to 1 micron, preferably of from 0.015 micron to 0.75 micron, more preferably of from 0.02 micron to 0.50 micron. The microspheres can also have a D50[vol] particle diameter of from 0.6 micron to 0.9 micron, preferably of from 0.08 micron to 0.9 micron, and more preferably between of from 0.08 micron to 0.9 micron, such as from 0.08 micron to 0.8 micron, or such as of from 0.8 micron to 0.6 micron. According to an embodiment, the microspheres can also have a D50[vol] particle diameter of from 0.1 micron to 1 micron, preferably of from 0.12 micron to 1 micron, and more preferably between of from 0.16 micron to 1 micron, such as of from 0.2 micron to 1 micron, or such as of from 0.08 micron to 0.4 micron. The terms “micron” and “microns” describe the size in micrometers [μm].

In further embodiments, which can be combined with other embodiments described herein, the pigments, which can be described as microspheres, can have a D90[vol] particle diameter of from 0.1 micron to 1 micron, preferably of from 0.2 micron to 1 micron, and more preferably between of from 0.3 micron to 1 micron, such as of from 0.3 micron to 0.9 micron, or such as of from 0.4 micron to 0.8 micron, or such as of from 0.5 micron to 0.9 micron.

In some embodiments described herein, the pigments, which can be described as microspheres, can have a D10[vol] particle diameter of from 0.02 micron to 0.3 micron, preferably of from 0.06 micron to 0.3 micron, more preferably of from 0.08 micron to 0.3 micron, such as of from 0.08 micron to 0.2 micron, or such as of from 0.1 micron to 0.2 micron, or such as 0.12 micron to 0.3 micron.

In embodiments described herein, the D10[vol] particle diameter can be of from 0.02 micron to 0.3 micron and the D90[vol] can be of from 0.3 micron to 1 micron. In further embodiments, the D10[vol] particle diameter can be of from 0.06 micron to 0.2 micron and the D90[vol] can be of from 0.4 micron to 1 micron.

The particle diameter is represented by D10, D50 and/or by D90, which is the median diameter by volume. D10, D50 and D90 is measured with a Malvern Mastersizer 2000, which is a laser diffraction particle sizer and it is measured according to ISO 13320:2009(en) with Hydro 2000G or Hydro 2000S where the dispersant is water or ethanol. Detection range is from 0.01 micron to 2000 micron. D50 is expressed as ×50 in ISO 13320:2009(en).

The term “D10,” as used herein refers, to the 10th percentile number- or volume-based median particle diameter, which is the diameter below which 10% by number or volume of the particle population is found. The term “D50,” as used herein refers, to the 50th percentile number- or volume-based median particle diameter, which is the diameter below which 50% by number or volume of the particle population is found. The term “D90,” as used herein refers, to the 90th percentile number- or volume-based median particle diameter, which is the diameter below which 90% by number or volume of the particle population is found. The number or volume measurement is indicated by [num] for number or [vol] for volume. If not indicated otherwise, the particle size is given as D10[vol], D50[vol], and D90[vol], respectively.

Laser diffraction measures particle size distributions by measuring the angular variation in intensity of light scattered as a laser beam passes through a dispersed particulate sample analyzer and the particle size is reported as a volume equivalent sphere diameter. A discussion of calculating D50 is provided in Barber et al, Pharmaceutical Development and Technology, 3(2), 153-161 (1998), which is incorporated herein by reference. Pigment microparticles having a D50[vol] particle diameter of less than 20 nm may enter the cuticles and are therefore difficult to remove. For scattering purposes, Pigment(s) having a D10[vol] particle diameter of at least 60 nm, or at least 80 nm can be used. Pigment(s) having a D50[vol] particle diameter of more than 1 micron typically do not sufficiently adhere onto hair fibers.

According to an embodiment, the particle size distribution, either relative to the number or volume of the particles, of the pigment microparticles can be at least bi-modal. A bi-modal particle size distribution has two distinct peaks which are spaced relative from, while tri-modal particle size distribution has three distinct peaks. The term “peak” means a local maximum of the distribution curve. The “distance” between two peaks, expressed relative to the particle size, can be at least 0.05 micron, preferably at least 0.1 micron, such as at least 0.2 micron. Providing an at least bi-modal particle size distribution allows to tailor the optical appearance of the colored hair. For example, the scattering properties varies with the particle size so that particles of different size scatter the light into different directions.

The at least bi-modal particle size distribution can be relative to pigment microparticles formed by the same pigment material. In addition to that or alternatively, the at least bi-model particle size distribution can be provided by pigment microparticles of different pigment material.

The size of pigment microparticles which can be described to have a 2-dimensional shape, and which are referred to as 2-dimensional microparticles can be determined by SEM. The size of 2-dimensional microparticles can also be determined by laser diffraction measurements. The particle size determined by laser diffraction is a mean size of the different dimensions of the 2-dimensional particles. The apparent D50[vol] particle diameter of 2-dimensional microparticles, as measured by SEM, can be from 0.5 micron to 50 microns, more preferably from 0.8 micron to 20 microns, more preferably from 1 micron to 15 microns, more preferably from 1.5 micron to 10 microns.

According to an embodiment, pigment particles are referred to as being microspheres can be used light-scattering and/or light absorbing purposes. Those particles, due to their pigment material, impart the hair with a specific color.

According to an embodiment, pigment particles are referred to as being 2-dimensional microparticles can be mainly used for light-reflecting and/or light absorbing purposes. Those particles, due to their pigment material, mainly reflect the light without significantly alter the color of the light.

The pigment microparticles can be light absorbing, but which for wavelengths of visible light provide negligible to low or no scattering. While not wishing to bound by any specific theory, it is believed that such pigments can provide more chromatic colors. Such pigment microparticles can have a D50[vol] value between about 0.001 micron and about 0.15 micron, between about 0.005 micron and about 0.1 micron or between about 0.010 micron and about 0.075 micron.

The pigment microparticles can be predominantly light scattering for wavelengths of visible light and provide low light absorption. While not wishing to bound by any specific theory, it is believed that such pigments can provide the visual effect of lightening the hair. Such pigment microparticles, which can be microspheres, can have a D50[vol] value between about 0.05 micron to about about 1 micron, between 0.08 micron to about 0.9 micron, between about 0.05 micron and about 0.75 micron, between about 0.1 micron and about 0.5 micron or about 0.15 micron and about 0.4 micron. Such materials can have a refractive index above 1.5, above 1.7 or above 2.0.

Pigments made from metal and metal like materials which can conduct electricity, and which can absorb light and re-emit the light out of the metal to give the appearance of strong reflectance. While not wishing to be bound by any specific theory, it is believed that the absorbed light will induce alternating electric currents on the metal surface, and that this currents immediately re-emit light out of the metal. Such pigment microparticles can be platelets, e.g., having a thickness that is substantially smaller than the planar dimension. For example about five, about 10 or even about 400 times smaller in thickness than in the planer. Such platelets can have a planar dimension less than about 30 nm, but with a thickness less than about 10 micron wide. This includes a ratio of 10000 to 30, or 333. Platelets larger in size, such as 50 microns are even available in this thickness of 10 microns, and so the ratios can even go up to 2000.

The pigment microparticles can be a composite formed by two different types of pigment microparticles. Examples include a composite of a 2-dimensional microparticle and at least one micro spherical particle (microsphere), a composite of different micro spherical particles, and a composite of different 2-dimensional particles. Composite particles formed by 2-dimensional microparticles to which micro spherical particles adhere provide an attractive alternative to a pure mixture of 2-dimensional microparticles and micro spherical particles. For example, a metallic 2-dimensional microparticle can carry one or more micro spherical particle such as one or more organic micro spherical particle. The micro spherical particles attached or bonded to the 2-dimensional microparticle can be formed of the same pigment material or can be formed of different pigment material. Composite microparticles formed of 2-dimensional microparticles and micro spherical particles can provide multiple functionality in one particle such as (metallic) reflectance and dielectric scattering, reflectance and absorption.

Pigment microparticles may be materials which are composite comprising a core of pigments made from metal and metal like materials which can conduct electricity, and which can absorb light and re-emit the light out of the metal to give the appearance of strong reflectance. While not wishing to be bound by any specific theory, it is believed that the absorbed light will induce alternating electric currents on the metal surface, and that this currents immediately re-emit light out of the metal. Upon this pigment light absorbing microparticles is immobilized. Such pigment microparticles can be platelets, e.g., having a thickness that is substantially smaller than the planar dimension. For example, five, ten or even 20 times smaller in thickness than in the planer. Such platelets can have a planer dimension less than 15 microns, but with a thickness less than 1 microns, more preferably with a planer dimension less than 12 microns but with a thickness less than 750 nm, even more preferably with a plan dimension less than 10 microns and a thickness less than 0.5 micron. The light absorbing microparticles can have D50 [vol] value between 0.001 micron and 0.15 micron, more preferably between 0.002 micron and 0.1 micron and even more preferable between 0.005 micron and 0.075 micron.

The light absorbing microparticles may also include dyes, pigments, or materials with color centers in the crystal structure, or photonic structures resulting in destructive or constructive interference, diffraction or other structures and materials mentioned in the book “The Physics and Chemistry of Color: the Fifteen Causes of Color”, 2^{nd} Edition by K. I. Nassau (ISBN 978-0-471-39106-7).

The pigment microparticles can be both light scattering and absorbing for wavelengths of visible light. While not wishing to bound by any specific theory, it is believed that such pigments can provide both some visual effect of lightening the hair. Such pigment microparticles can have a D50[num] value between about 50 nm and about 750 nm, between about 100 nm and about 500 nm or between about 150 nm and about 400 nm. Such materials have a refractive index above about 1.5, above about 1.7 or above about 2.0.

According to an embodiment, different pigment microparticles are combined to provide reflective, transmitting and refractive properties of the hair colored with the color composition described herein. A microparticle combination can be a material composite using at least two different pigment materials to form the pigment microparticles. In addition to, or alternating to, the microparticle combination, a mixture of separate pigment microparticles of different type can be used to bring about the desired reflective, transmitting and refractive properties.

The composite pigments, combination of pigments, and mixtures of pigment microparticles eliminate, or at least significantly reduce, hair penetration and scattering by light and thus eliminate the perception of pigment of natural hair color change.

### Pigment Concentration

The color composition for coloring hair fibers according to the present disclosure comprises microparticles comprising at least one pigment. The color composition comprises from about 0.01% to about 40%, about 0.05% to about 35%, about 0.1 to about 25%, or about 0.15% and about 20% pigment(s), by weight of the color composition.

### Pigment Material

The material of the pigment microparticles can be inorganic or organic. Inorganic-organic mixed pigments are also possible.

According to an embodiment, inorganic pigment(s) are used. The advantage of inorganic pigment(s) is their excellent resistance to light, weather, and temperature. The inorganic pigment(s) can be of natural origin, and are, for example, derived from material selected from the group consisting of chalk, ochre, umber, green earth, burnt sienna, and graphite. The pigment(s) can preferably be white pigments, such as, for example, titanium dioxide or zinc oxide. The pigment(s) can also be colored pigments, such as, for example, ultramarine or iron oxide red, luster pigments, metal effect pigments, pearlescent pigments, and fluorescent or phosphorescent pigments. The pigment(s) can be selected from the group consisting of metal oxides, hydroxides and oxide hydrates, mixed phase pigments, sulfur-containing silicates, metal sulfides, complex metal cyanides, metal sulfates, chromates and molybdates, alloys, and the metals themselves. The pigment(s) can be selected from the group consisting of titanium dioxide (CI 77891), black iron oxide (CI 77499), yellow iron oxide (CI 77492), red and brown iron oxide (CI 77491), manganese violet (CI 77742), ultramarine (sodium aluminum sulfosilicates, CI 77007, Pigment Blue 29), chromium oxide hydrate (CI 77289), Prussian blue (ferric ferrocyanide, CI 77510), carmine (cochineal), zinc sulfide, barium sulfate, zinc oxide, siliconised titanium dioxide, siliconised zinc sulfide, siliconised zinc oxide, and mixtures thereof. The pigment(s) can be selected from the group consisting of iron oxide, titanium dioxide, mica, borosilicate, and combinations thereof. The pigment(s) can comprise an iron oxide (Fe₂O₃) pigment. The pigment(s) can comprise a combination of mica and titanium dioxide.

The pigment(s) can be pearlescent and colored pigment(s), and can preferably be based on mica which are coated with a metal oxide or a metal oxychloride, such as titanium dioxide or bismuth oxychloride, and optionally further color-imparting substances, such as iron oxides, Prussian blue, ultramarine, and carmine. The color exhibited by a pigment can be adjusted by varying the layer thickness. Such pigments are sold, for example, under the trade names Rona®, Colorona®, Dichrona®, RonaFlair®, Ronastar®, Xirona® and Timiron® all of which are available from Merck, Darmstadt, Germany. For example, Xirona® is a brand for color travel pigments that display color shifting effects depending on the viewing angle and are based on either natural mica, SiO₂ or calcium aluminum borosilicate flakes, coated with varying layers of TiO₂. Pigment(s) from the line KTZ® from Kobo Products, Inc., 3474 So. Clinton Ave., So. Plainfield, USA, are also useful herein, in particular the Surface Treated KTZ® Pearlescent Pigments from Kobo. Particularly useful are KTZ® FINE WHITE (mica and TiO₂) having a D50 particle diameter of 5 to 25 micron and also KTZ® CELESTIAL LUSTER (mica and TiO2, 10 to 60 micron) as well as KTZ® CLASSIC WHITE (mica and TiO2, 10 to 60 micron). Also useful are SynCrystal Sapphire from Eckart Effect Pigments, which is a blue powder comprising platelets of synthetic fluorphlogopite coated with titanium dioxide, ferric ferrocyanide and small amounts of tin oxide. Also useful is SYNCRYSTAL Almond also from Eckart, which is a beige powder with a copper reflection color and is composed of platelets of synthetic fluorphlogopite and coated with titanium dioxide and iron oxides. Also useful is Duocrome® RV 524C from BASF, which provides a two color look via a lustrous red powder with a violet reflection powder due to its composition of mica, titanium dioxide and carmine. The colored pigment(s) can be lightly bright colored pigment(s) and can particularly be white color variations.

The pigment(s) can be organic pigments. The at least one pigment can be an organic pigment. As used herein, the term “organic pigment” means any pigment that satisfies the definition in Ullmann's encyclopedia in the chapter on organic pigments. For instance, the at least one organic pigment can be chosen from nitroso, nitro, azo, xanthene, quinoline, anthraquinone, phthalocyanin, copper phthalocyanin, copper hexadecachlorophthalocyanine, 2-[(2-Methoxy-4-nitrophenyl)azo]-N-(2-methoxyphenyl)-3-oxobutyramide, metal-complex, isoindolinone, isoindoline, quinacridone, perinone, perylene, diketopyrrolopyrrole, thioindigo, dioxazine, triphenylmethane, dimethylquinacridone and quinophthalone compounds, Azo-dyes, Nonionic azo dyes, Anionic Azo dyes, Cationic azo dyes, Complex forming azo dye, aza annulene dyes, aza analogue of diarylmethane dyes, aza annulene dyes, Nitro-dyes and their pigments, Carbonyl dyes and their pigments (for example, Anthrachinon dyes, indigo), Sulfur dyes, Florescence dyes, Anthracene or Insoluble alkali or earth metal acid dyes.

Or the pigment can be at least one of uncolored and UV absorbing.

The organic pigment(s) can be selected from the group consisting of natural pigments sepia, gamboge, bone charcoal, Cassel brown, indigo, chlorophyll and other plant pigments. The synthetic organic pigments can be selected from the group consisting of azo pigments, anthraquinoids, indigoids, dioxazine, quinacridone, phthalocyanine, isoindolinone, perylene and perinone, metal complex, alkali blue, diketopyrrolopyrrole pigments, and combinations thereof. A particularly preferred pigment is 7-Bis(1,3-dichloropropan-2-yl)benzo[lmn][3,8]phenanthrolin-1,3,6,8(2H,7H)-tetraon.

According to an embodiment, the pigment(s) can be selected from any of the pigments and groups listed below including any combination thereof (with CI meaning color index and CAS meaning Chemical Abstract Service Number).

These pigments include but are not limited to: Pigment Black 10 [C.I. 77265, (CAS: 7782-42-5)], Pigment Black 11 [C.I. 77499, (CAS: 12227-89-3)], Pigment Black 12 [C.I. 77543, (CAS: 68187-02-0)], Pigment Black 13 [C.I. 77322, (CAS: 1307-96-6)], Pigment Black 14 [C.I. 77728, (CAS: 83512-98-5)], Pigment Black 15 [C.I. 77403, (CAS: 1317-38-0)], Pigment Black 17 [C.I. 77975, (CAS: 1314-98-3)], Pigment Black 18 [C.I. 77011, (CAS: 12001-98-8)], Pigment Black 23 [C.I. 77865, (CAS: 68187-54-2)], Pigment Black 24 [C.I. 77898, (CAS: 68187-00-8)], Pigment Black 25 [C.I. 77332, (CAS: 68186-89-0)], Pigment Black 26 [C.I. 77494, (CAS: 68186-94-7)], Pigment Black 27 [C.I. 77502, (CAS: 68186-97-0)], Pigment Black 28 [C.I. 77428, (CAS: 68186-91-4)], Pigment Black 29 [C.I. 77498, (CAS: 68187-50-8)], Pigment Black 30 [C.I. 77504, (CAS: 71631-15-7)], Pigment Black 31 [C.I. 71132, (CAS: 67075-37-0)], Pigment Black 32 [C.I. 71133, (CAS: 83524-75-8)], Pigment Black 33 [C.I. 77537, (CAS: 188735-18-4)], Pigment Black 34 [C.I. 77770, (CAS: 1317-33-5)], Pigment Black 6 [C.I. 77266, (CAS: 1333-86-4)], Pigment Black 7 [C.I. 77266, (CAS: 1333-86-4)], Pigment Black 8 [C.I. 77268, (CAS: 1339-82-8)], Pigment Black 9 [C.I. 77267, (CAS: 8021-99-6)], Pigment Blue 10 [C.I. 44040, (CAS: 1325-93-5)], Pigment Blue 15 [C.I. 74160, (CAS: 147-14-8)], Pigment Blue 16 [C.I. 74100, (CAS: 574-93-6)], Pigment Blue 18 [C.I. 42770, (CAS: 1324-77-2)], Pigment Blue 21 [C.I. 69835, (CAS: 1324-26-1)], Pigment Blue 22 [C.I. 69810, (CAS: 1324-27-2)], Pigment Blue 25 [C.I. 21180, (CAS: 10127-03-4)], Pigment Blue 26 [C.I. 21185, (CAS: 5437-88-7)], Pigment Blue 28 [C.I. 77346, (CAS: 1345-16-0)], Pigment Blue 29 [C.I. 77007, (CAS: 57455-37-5)], Pigment Blue 30 [C.I. 77420, (CAS: 1339-83-9)], Pigment Blue 32 [C.I. 77365, (CAS: 69458-70-4)], Pigment Blue 33 [C.I. 77112, (CAS: 8046-59-1)], Pigment Blue 34 [C.I. 77450, (CAS: 1317-40-4)], Pigment Blue 35 [C.I. 77368, (CAS: 83712-59-8)], Pigment Blue 36 [C.I. 77343, (CAS: 68187-11-1)], Pigment Blue 56 [C.I. 42800, (CAS: 6417-46-5)], Pigment Blue 57 [C.I. 42795, (CAS: 5905-38-4)], Pigment Blue 60 [C.I. 69800, (CAS: 81-77-6)], Pigment Blue 61 [C.I. 42765, (CAS: 1324-76-1)], Pigment Blue 62 [C.I. 42595, (CAS: 82338-76-9)], Pigment Blue 63 [C.I. 73015, (CAS: 16521-38-3)], Pigment Blue 64 [C.I. 69825, (CAS: 130-20-1)], Pigment Blue 65 [C.I. 59800, (CAS: 116-71-2)], Pigment Blue 66 [C.I. 73000, (CAS: 482-89-3)], Pigment Blue 71 [C.I. 77998, (CAS: 68186-95-8)], Pigment Blue 72 [C.I. 77347, (CAS: 68186-87-8)], Pigment Blue 73 [C.I. 77364, (CAS: 68187-40-6)], Pigment Blue 74 [C.I. 77366, (CAS: 68412-74-8)], Pigment Blue 75 [C.I. 74160, (CAS: 3317-67-7)], Pigment Blue 76 [C.I. 742520, (CAS: 176365-61-0)], Pigment Blue 78 [C.I. 42090, (CAS: 68921-42-6)], Pigment Blue 79 [C.I. 741300, (CAS: 14154-42-8)], Pigment Blue 9 [C.I. 42025B, (CAS: 596-42-9)], Pigment Brown 1 [C.I. 12480, (CAS: 6410-40-8)], Pigment Brown 10 [C.I. 77227, (CAS: 12013-69-3)], Pigment Brown 11 [C.I. 77495, (CAS: 64294-89-9)], Pigment Brown 2 [C.I. 12071, (CAS: 10279-43-3)], Pigment Brown 22 [C.I. 10407, (CAS: 29398-96-7)], Pigment Brown 23 [C.I. 20060, (CAS: 35869-64-8)], Pigment Brown 24 [C.I. 77310, (CAS: 68186-90-3)], Pigment Brown 26 [C.I. 71129, (CAS: 81-33-4)], Pigment Brown 27 [C.I. 73410, (CAS: 3989-75-1)], Pigment Brown 28 [C.I. 69015, (CAS: 131-92-0)], Pigment Brown 33 [C.I. 77503, (CAS: 68186-88-9)], Pigment Brown 34 [C.I. 77497, (CAS: 68187-10-0)], Pigment Brown 35 [C.I. 77501, (CAS: 68187-09-7)], Pigment Brown 37 [C.I. 77890, (CAS: 70248-09-8)], Pigment Brown 38 [C.I. 561660, (CAS: 126338-72-5)], Pigment Brown 39 [C.I. 77312, (CAS: 71750-83-9)], Pigment Brown 6 [C.I. 77491, 77492 and 77499, (CAS: 52357-70-7)], Pigment Brown 9 [C.I. 77430, (CAS: 8014-85-5)], Pigment Green 10 [C.I. 12775, (CAS: 61725-51-7)], Pigment Green 12 [C.I. 10020, (CAS: 84682-41-7)], Pigment Green 15 [C.I. 77600, (CAS: 12224-92-9)], Pigment Green 17 [C.I. 77288, (CAS: 1308-38-9)], Pigment Green 18 [C.I. 77289, (CAS: 12001-99-9)], Pigment Green 19 [C.I. 77335, (CAS: 8011-87-8)], Pigment Green 20 [C.I. 77408, (CAS: 8007-61-2)], Pigment Green 21 [C.I. 77410, (CAS: 12002-03-8)], Pigment Green 22 [C.I. 77412, (CAS: 1345-20-6)], Pigment Green 23 [C.I. 77009, (CAS: 1344-98-5)], Pigment Green 24 [C.I. 77013, (CAS: 1345-00-2)], Pigment Green 26 [C.I. 77344, (CAS: 68187-49-5)], Pigment Green 27 [C.I. 77520, (CAS: 15418-51-6)], Pigment Green 36 [C.I. 74265, (CAS: 14302-13-7)], Pigment Green 37 [C.I. 74255, (CAS: 1330-37-6)], Pigment Green 38 [C.I. 74265, (CAS: 14302-13-7)], Pigment Green 42 [C.I. 74260, (CAS: 1328-53-6)], Pigment Green 47 [C.I. 59825, (CAS: 128-58-5)], Pigment Green 50 [C.I. 77377, (CAS: 68186-85-6)], Pigment Green 51 [C.I. 77300, (CAS: 68553-01-5)], Pigment Green 54 [C.I. 59830, (CAS: 25704-81-8)], Pigment Green 58 [C.I. 742655, (CAS: 1143572-73-9)], Pigment Green 8 [C.I. 10006, (CAS: 16143-80-9)], Pigment Green 9 [C.I. 49415, (CAS: 1326-13-2)], Pigment Orange 1 [C.I. 11725, (CAS: 6371-96-6)], Pigment Orange 13 [C.I. 21110, (CAS: 3520-72-7)], Pigment Orange 14 [C.I. 21165, (CAS: 6837-37-2)], Pigment Orange 15 [C.I. 21130, (CAS: 6358-88-9)], Pigment Orange 16 [C.I. 21160, (CAS: 6505-28-8)], Pigment Orange 17 [C.I. 15510, (CAS: 15782-04-4)], Pigment Orange 17 [C.I. 15510, (CAS: 15876-51-4)], Pigment Orange 18 [C.I. 15970, (CAS: 1325-14-0)], Pigment Orange 19 [C.I. 15990, (CAS: 5858-88-8)], Pigment Orange 20 [C.I. 77202, (CAS: 12656-57-4)], Pigment Orange 21 [C.I. 77601, (CAS: 1344-38-3)], Pigment Orange 22 [C.I. 12470, (CAS: 6358-48-1)], Pigment Orange 23 [C.I. 77201, (CAS: 1345-09-1)], Pigment Orange 24 [C.I. 12305, (CAS: 6410-27-1)], Pigment Orange 3 [C.I. 12105, (CAS: 6410-15-7)], Pigment Orange 31 [C.I. 20050, (CAS: 5280-74-0)], Pigment Orange 34 [C.I. 21115, (CAS: 15793-73-4)], Pigment Orange 39 [C.I. 45370, (CAS: 15876-57-0)], Pigment Orange 4 [C.I. 12459, (CAS: 21889-27-0)], Pigment Orange 40 [C.I. 59700, (CAS: 128-70-1)], Pigment Orange 43 [C.I. 71105, (CAS: 4424-06-0)], Pigment Orange 44 [C.I. 21162, (CAS: 17453-73-5)], Pigment Orange 45 [C.I. 77601, (CAS: 59519-55-0)], Pigment Orange 46 [C.I. 15602, (CAS: 63467-26-5)], Pigment Orange 5 [C.I. 12075, (CAS: 3468-63-1)], Pigment Orange 6 [C.I. 12730, (CAS: 6407-77-8)], Pigment Orange 61 [C.I. 11265, (CAS: 40716-47-0)], Pigment Orange 64 [C.I. 12760, (CAS: 72102-84-2)], Pigment Orange 65 [C.I. 48053, (CAS: 20437-10-9)], Pigment Orange 66 [C.I. 48210, (CAS: 68808-69-5)], Pigment Orange 67 [C.I. 12915, (CAS: 74336-59-7)], Pigment Orange 68 [C.I. 486150, (CAS: 42844-93-9)], Pigment Orange 69 [C.I. 56292, (CAS: 85959-60-0)], Pigment Orange 7 [C.I. 15530, (CAS: 5850-81-7)], Pigment Orange 71 [C.I. 561200, (CAS: 84632-50-8)], Pigment Orange 72 [C.I. 211095, (CAS: 384329-80-0)], Pigment Orange 73 [C.I. 561170, (CAS: 84632-59-7)], Pigment Orange 75 [C.I. 772830, (CAS: 12014-93-6)], Pigment Orange 77 [C.I. 59105, (CAS: 1324-11-4)], Pigment Red 10 [C.I. 12440, (CAS: 6410-35-1)], Pigment Red 100 [C.I. 13058, (CAS: 6371-55-7)], Pigment Red 101 [C.I. 77491, (CAS: 1309-37-1)], Pigment Red 101 [C.I. 77015, (CAS: 529484-30-8)], Pigment Red 103 [C.I. 77601, (CAS: 59519-56-1)], Pigment Red 104 [C.I. 77605, (CAS: 12656-85-8)], Pigment Red 105 [C.I. 77578, (CAS: 1314-41-6)], Pigment Red 106 [C.I. 77766, (CAS: 1344-48-5)], Pigment Red 107 [C.I. 77060, (CAS: 1345-04-6)], Pigment Red 108 [C.I. 77202, (CAS: 58339-34-7)], Pigment Red 109 [C.I. 77482, (CAS: 1345-24-0)], Pigment Red 11 [C.I. 12430, (CAS: 6535-48-4)], Pigment Red 112 [C.I. 12370, (CAS: 6535-46-2)], Pigment Red 113 [C.I. 77201, (CAS: 1345-09-1)], Pigment Red 114 [C.I. 12351, (CAS: 6358-47-0)], Pigment Red 115 [C.I. 15851, (CAS: 6358-40-3)], Pigment Red 117 [C.I. 15603, (CAS: 10142-77-5)], Pigment Red 119 [C.I. 12469, (CAS: 72066-77-4)], Pigment Red 12 [C.I. 12385, (CAS: 6410-32-8)], Pigment Red 121 [C.I. 77302, (CAS: 12125-42-7)], Pigment Red 122 [C.I. 73915, (CAS: 980-26-7)], Pigment Red 13 [C.I. 12395, (CAS: 6535-47-3)], Pigment Red 133 [C.I. 15920, (CAS: 5280-67-1)], Pigment Red 14 [C.I. 12380, (CAS: 6471-50-7)], Pigment Red 141 [C.I. 20044, (CAS: 3864-06-0)], Pigment Red 144 [C.I. 20735, (CAS: 5280-78-4)], Pigment Red 146 [C.I. 12485, (CAS: 5280-68-2)], Pigment Red 147 [C.I. 12433, (CAS: 68227-78-1)], Pigment Red 148 [C.I. 12369, (CAS: 94276-08-1)], Pigment Red 149 [C.I. 71137, (CAS: 4948-15-6)], Pigment Red 15 [C.I. 12465, (CAS: 6410-39-5)], Pigment Red 150 [C.I. 12290, (CAS: 56396-10-2)], Pigment Red 151 [C.I. 15892, (CAS: 61013-97-6)], Pigment Red 157 [C.I. 12355, (CAS: 6471-49-4)], Pigment Red 16 [C.I. 12500, (CAS: 6407-71-2)], Pigment Red 162 [C.I. 12431, (CAS: 6358-59-4)], Pigment Red 163 [C.I. 12455, (CAS: 6410-37-3)], Pigment Red 164 [C.I. 212855, (CAS: 72659-69-9)], Pigment Red 166 [C.I. 20730, (CAS: 3905-19-9)], Pigment Red 168 [C.I. 59300, (CAS: 4378-61-4)], Pigment Red 169 [C.I. 45160, (CAS: 12237-63-7)], Pigment Red 17 [C.I. 12390, (CAS: 6655-84-1)], Pigment Red 170 [C.I. 12475, (CAS: 2786-76-7)], Pigment Red 170 [C.I. 12474, (CAS: 36968-27-1)], Pigment Red 171 [C.I. 12512, (CAS: 6985-95-1)], Pigment Red 172 [C.I. 45430, (CAS: 12227-78-0)], Pigment Red 173 [C.I. 45170, (CAS: 12227-77-9)], Pigment Red 174 [C.I. 45410, (CAS: 15876-58-1)], Pigment Red 175 [C.I. 12513, (CAS: 6985-92-8)], Pigment Red 177 [C.I. 65300, (CAS: 4051-63-2)], Pigment Red 179 [C.I. 71130, (CAS: 5521-31-3)], Pigment Red 18 [C.I. 12350, (CAS: 3564-22-5)], Pigment Red 181 [C.I. 73360, (CAS: 2379-74-0)], Pigment Red 184 [C.I. 12487, (CAS: 99402-80-9)], Pigment Red 185 [C.I. 12516, (CAS: 51920-12-8)], Pigment Red 187 [C.I. 12486, (CAS: 59487-23-9)], Pigment Red 188 [C.I. 12467, (CAS: 61847-48-1)], Pigment Red 189 [C.I. 71135, (CAS: 2379-77-3)], Pigment Red 19 [C.I. 12400, (CAS: 6410-33-9)], Pigment Red 190 [C.I. 71140, (CAS: 6424-77-7)], Pigment Red 192 [C.I. 739155, (CAS: 61968-81-8)], Pigment Red 193 [C.I. 16185, (CAS: 12227-62-2)], Pigment Red 195 [C.I. 70320, (CAS: 4203-77-4)], Pigment Red 196 [C.I. 67000, (CAS: 2379-79-5)], Pigment Red 198 [C.I. 73390, (CAS: 6371-31-9)], Pigment Red 2 [C.I. 12310, (CAS: 6041-94-7)], Pigment Red 200 [C.I. 15867, (CAS: 58067-05-3)], Pigment Red 200 [C.I. 15867, (CAS: 32041-58-0)], Pigment Red 202 [C.I. 73907, (CAS: 3089-17-6)], Pigment Red 208 [C.I. 12514, (CAS: 31778-10-6)], Pigment Red 21 [C.I. 12300, (CAS: 6410-26-0)], Pigment Red 210 [C.I. 12477, (CAS: 61932-63-6)], Pigment Red 211 [C.I. 15910, (CAS: 85702-54-1)], Pigment Red 212 [C.I. 12360, (CAS: 6448-96-0)], Pigment Red 214 [C.I. 200660, (CAS: 40618-31-3)], Pigment Red 216 [C.I. 59710, (CAS: 1324-33-0)], Pigment Red 22 [C.I. 12315, (CAS: 6448-95-9)], Pigment Red 220 [C.I. 20055, (CAS: 68259-05-2)], Pigment Red 221 [C.I. 20065, (CAS: 71566-54-6)], Pigment Red 222 [C.I. 123665, (CAS: 20981-12-8)], Pigment Red 224 [C.I. 71127, (CAS: 128-69-8)], Pigment Red 226 [C.I. 597200, (CAS: 72828-01-4)], Pigment Red 229 [C.I. 77006, (CAS: 85536-78-3)], Pigment Red 230 [C.I. 77003, (CAS: 68187-27-9)], Pigment Red 231 [C.I. 77005, (CAS: 68186-99-2)], Pigment Red 232 [C.I. 77996, (CAS: 68412-79-3)], Pigment Red 233 [C.I. 77301, (CAS: 68187-12-2)], Pigment Red 235 [C.I. 77290, (CAS: 68201-65-0)], Pigment Red 236 [C.I. 77863, (CAS: 68187-53-1)], Pigment Red 242 [C.I. 20067, (CAS: 52238-92-3)], Pigment Red 243 [C.I. 15910, (CAS: 50326-33-5)], Pigment Red 243 [C.I. 15910, (CAS: 431991-58-1)], Pigment Red 247 [C.I. 15915, (CAS: 43035-18-3)], Pigment Red 248 [C.I. 200552, (CAS: 80648-58-4)], Pigment Red 251 [C.I. 12925, (CAS: 74336-60-0)], Pigment Red 253 [C.I. 12375, (CAS: 85776-13-2)], Pigment Red 254 [C.I. 56110, (CAS: 84632-65-5)], Pigment Red 255 [C.I. 561050, (CAS: 54660-00-3)], Pigment Red 256 [C.I. 124635, (CAS: 79102-65-1)], Pigment Red 257 [C.I. 562700, (CAS: 70833-37-3)], Pigment Red 258 [C.I. 12318, (CAS: 57301-22-1)], Pigment Red 259 [C.I. 77007, (CAS: 113956-14-2)], Pigment Red 260 [C.I. 56295, (CAS: 71552-60-8)], Pigment Red 261 [C.I. 12468, (CAS: 16195-23-6)], Pigment Red 264 [C.I. 561300, (CAS: 88949-33-1)], Pigment Red 265 [C.I. 772830, (CAS: 12014-93-6)], Pigment Red 267 [C.I. 12396, (CAS: 68016-06-8)], Pigment Red 268 [C.I. 12316, (CAS: 16403-84-2)], Pigment Red 269 [C.I. 12466, (CAS: 67990-05-0)], Pigment Red 271 [C.I. 487100, (CAS: 85958-80-1)], Pigment Red 273 [C.I. 16035, (CAS: 68583-95-9)], Pigment Red 274 [C.I. 16255, (CAS: 12227-64-4)], Pigment Red 3 [C.I. 12120, (CAS: 2425-85-6)], Pigment Red 30 [C.I. 12330, (CAS: 6471-48-3)], Pigment Red 32 [C.I. 12320, (CAS: 6410-29-3)], Pigment Red 37 [C.I. 21205, (CAS: 6883-91-6)], Pigment Red 38 [C.I. 21120, (CAS: 6358-87-8)], Pigment Red 39 [C.I. 21080, (CAS: 6492-54-2)], Pigment Red 4 [C.I. 12085, (CAS: 2814-77-9)], Pigment Red 40 [C.I. 12170, (CAS: 2653-64-7)], Pigment Red 41 [C.I. 21200, (CAS: 6505-29-9)], Pigment Red 42 [C.I. 21210, (CAS: 6358-90-3)], Pigment Red 48 [C.I. 15865, (CAS: 3564-21-4)], Pigment Red 48 [C.I. 15865, (CAS: 1325-12-8)], Pigment Red 48 [C.I. 15865, (CAS: 7585-41-3)], Pigment Red 48 [C.I. 15865, (CAS: 7023-61-2)], Pigment Red 48 [C.I. 15865, (CAS: 15782-05-5)], Pigment Red 48 [C.I. 15865, (CAS: 5280-66-0)], Pigment Red 48 [C.I. 15865, (CAS: 71832-83-2)], Pigment Red 48 [C.I. 15865, (CAS: 68966-97-2)], Pigment Red 49 [C.I. 15630, (CAS: 1248-18-6)], Pigment Red 49 [C.I. 15630, (CAS: 1325-06-0)], Pigment Red 49 [C.I. 15630, (CAS: 1103-38-4)], Pigment Red 49 [C.I. 15630, (CAS: 1103-39-5)], Pigment Red 49 [C.I. 15630, (CAS: 6371-67-1)], Pigment Red 5 [C.I. 12490, (CAS: 6410-41-9)], Pigment Red 50 [C.I. 15500, (CAS: 5850-76-0)], Pigment Red 50 [C.I. 15500, (CAS: 6372-81-2)], Pigment Red 51 [C.I. 15580, (CAS: 5850-87-3)], Pigment Red 52 [C.I. 15860, (CAS: 5858-82-2)], Pigment Red 52 [C.I. 15860, (CAS: 1325-11-7)], Pigment Red 52 [C.I. 15860, (CAS: 17852-99-2)], Pigment Red 52 [C.I. 15860, (CAS: 17814-20-9)], Pigment Red 52 [C.I. 15860, (CAS: 12238-31-2)], Pigment Red 53 [C.I. 15585, (CAS: 2092-56-0)], Pigment Red 53 [C.I. 15585, (CAS: 1325-04-8)], Pigment Red 53 [C.I. 15585, (CAS: 67990-35-6)], Pigment Red 53 [C.I. 15585, (CAS: 73263-40-8)], Pigment Red 54 [C.I. 14830, (CAS: 6373-10-0)], Pigment Red 55 [C.I. 15820, (CAS: 141052-43-9)], Pigment Red 57 [C.I. 15850, (CAS: 5858-81-1)], Pigment Red 57 [C.I. 15850, (CAS: 17852-98-1)], Pigment Red 57 [C.I. 15850, (CAS: 55491-44-6)], Pigment Red 58 [C.I. 15825, (CAS: 1325-09-3)], Pigment Red 58 [C.I. 15825, (CAS: 7538-59-2)], Pigment Red 58 [C.I. 15825, (CAS: 15782-03-3)], Pigment Red 58 [C.I. 15825, (CAS: 76613-71-3)], Pigment Red 58 [C.I. 15825, (CAS: 64552-28-9)], Pigment Red 6 [C.I. 12090, (CAS: 6410-13-5)], Pigment Red 60 [C.I. 16105, (CAS: 15782-06-6)], Pigment Red 60 [C.I. 16105, (CAS: 1325-16-2)], Pigment Red 61 [C.I. 24830, (CAS: 1325-29-7)], Pigment Red 62 [C.I. 23295, (CAS: 109823-18-9)], Pigment Red 63 [C.I. 15880, (CAS: 21416-46-6)], Pigment Red 63 [C.I. 15880, (CAS: 6417-83-0)], Pigment Red 63 [C.I. 15880, (CAS: 15792-20-8)], Pigment Red 63 [C.I. 15880, (CAS: 35355-77-2)], Pigment Red 64 [C.I. 15800, (CAS: 16508-79-5)], Pigment Red 64 [C.I. 15800, (CAS: 6371-76-2)], Pigment Red 65 [C.I. 18020, (CAS: 1325-21-9)], Pigment Red 66 [C.I. 18000, (CAS: 1325-19-5)], Pigment Red 67 [C.I. 18025, (CAS: 1325-22-0)], Pigment Red 68 [C.I. 15525, (CAS: 5850-80-6)], Pigment Red 69 [C.I. 15595, (CAS: 5850-90-8)], Pigment Red 7 [C.I. 12420, (CAS: 6471-51-8)], Pigment Red 70 [C.I. 15590, (CAS: 5850-89-5)], Pigment Red 77 [C.I. 15826, (CAS: 6358-39-0)], Pigment Red 8 [C.I. 12335, (CAS: 6410-30-6)], Pigment Red 83 [C.I. 58000, (CAS: 104074-25-1)], Pigment Red 84 [C.I. 58210, (CAS: 1328-07-0)], Pigment Red 85 [C.I. 63350, (CAS: 6370-96-3)], Pigment Red 86 [C.I. 73375, (CAS: 6371-26-2)], Pigment Red 89 [C.I. 60745, (CAS: 6409-74-1)], Pigment Red 9 [C.I. 12460, (CAS: 6410-38-4)], Pigment Red 90 [C.I. 45380, (CAS: 15876-39-8)], Pigment Red 93 [C.I. 12152, (CAS: 6548-36-3)], Pigment Red 95 [C.I. 15897, (CAS: 72639-39-5)], Pigment Red 99 [C.I. 15570, (CAS: 5850-85-1)], Pigment Violet 10 [C.I. 42535, (CAS: 1325-82-2)], Pigment Violet 12 [C.I. 58050, (CAS: 1328-03-6)], Pigment Violet 13 [C.I. 125085, (CAS: 83399-83-1)], Pigment Violet 14 [C.I. 77360, (CAS: 10101-56-1)], Pigment Violet 15 [C.I. 77007, (CAS: 12769-96-9)], Pigment Violet 16 [C.I. 77742, (CAS: 10101-66-3)], Pigment Violet 19 [C.I. 46500, (CAS: 1047-16-1)], Pigment Violet 20 [C.I. 58225, (CAS: 6486-92-6)], Pigment Violet 23 [C.I. 51319, (CAS: 215247-95-3)], Pigment Violet 25 [C.I. 12321, (CAS: 6358-46-9)], Pigment Violet 27 [C.I. 42535, (CAS: 12237-62-6)], Pigment Violet 29 [C.I. 71129, (CAS: 81-33-4)], Pigment Violet 3 [C.I. 42535, (CAS: 68647-35-8)], Pigment Violet 3 [C.I. 42535, (CAS: 68308-41-8)], Pigment Violet 3 [C.I. 42535, (CAS: 67989-22-4)], Pigment Violet 31 [C.I. 60010, (CAS: 1324-55-6)], Pigment Violet 33 [C.I. 60005, (CAS: 1324-17-0)], Pigment Violet 36 [C.I. 73385, (CAS: 5462-29-3)], Pigment Violet 37 [C.I. 51345, (CAS: 17741-63-8)], Pigment Violet 38 [C.I. 73395, (CAS: 2379-75-1)], Pigment Violet 47 [C.I. 77363, (CAS: 68610-13-9)], Pigment Violet 48 [C.I. 77352, (CAS: 68608-93-5)], Pigment Violet 49 [C.I. 77362, (CAS: 16827-96-6)], Pigment Violet 5 [C.I. 58055, (CAS: 1328-04-7)], Pigment Violet 6 [C.I. 58060, (CAS: 6483-85-8)], Pigment Violet 6 [C.I. 58060, (CAS: 1328-05-8)], Pigment Violet 7 [C.I. 58065, (CAS: 1328-06-9)], Pigment Violet 8 [C.I. 18005, (CAS: 1325-20-8)], Pigment Yellow 1 [C.I. 11680, (CAS: 2512-29-0)], Pigment Yellow 10 [C.I. 12710, (CAS: 6407-75-6)], Pigment Yellow 100 [C.I. 19140, (CAS: 12225-21-7)], Pigment Yellow 104 [C.I. 15985, (CAS: 15790-07-5)], Pigment Yellow 105 [C.I. 11743, (CAS: 12236-75-8)], Pigment Yellow 109 [C.I. 56284, (CAS: 5045-40-9)], Pigment Yellow 11 [C.I. 10325, (CAS: 2955-16-0)], Pigment Yellow 110 [C.I. 56280, (CAS: 5590-18-1)], Pigment Yellow 111 [C.I. 11745, (CAS: 15993-42-7)], Pigment Yellow 112 [C.I. 70600, (CAS: 475-71-8)], Pigment Yellow 114 [C.I. 21092, (CAS: 68610-87-7)], Pigment Yellow 115 [C.I. 47005, (CAS: 68814-04-0)], Pigment Yellow 116 [C.I. 11790, (CAS: 61968-84-1)], Pigment Yellow 117 [C.I. 48043, (CAS: 21405-81-2)], Pigment Yellow 118 [C.I. 77894, (CAS: 61512-65-0)], Pigment Yellow 119 [C.I. 77496, (CAS: 68187-51-9)], Pigment Yellow 12 [C.I. 21090, (CAS: 6358-85-6)], Pigment Yellow 123 [C.I. 65049, (CAS: 4028-94-8)], Pigment Yellow 124 [C.I. 21107, (CAS: 67828-22-2)], Pigment Yellow 126 [C.I. 21101, (CAS: 90268-23-8)], Pigment Yellow 127 [C.I. 21102, (CAS: 68610-86-6)], Pigment Yellow 128 [C.I. 20037, (CAS: 79953-85-8)], Pigment Yellow 129 [C.I. 48042, (CAS: 15680-42-9)], Pigment Yellow 13 [C.I. 21100, (CAS: 5102-83-0)], Pigment Yellow 130 [C.I. 117699, (CAS: 23739-66-4)], Pigment Yellow 133 [C.I. 139395, (CAS: 85702-53-0)], Pigment Yellow 134 [C.I. 21111, (CAS: 31775-20-9)], Pigment Yellow 138 [C.I. 56300, (CAS: 30125-47-4)], Pigment Yellow 139 [C.I. 56298, (CAS: 36888-99-0)], Pigment Yellow 14 [C.I. 21095, (CAS: 5468-75-7)], Pigment Yellow 147 [C.I. 60645, (CAS: 4118-16-5)], Pigment Yellow 148 [C.I. 50600, (CAS: 20572-37-6)], Pigment Yellow 15 [C.I. 21220, (CAS: 6528-35-4)], Pigment Yellow 150 [C.I. 12764, (CAS: 872613-79-1)], Pigment Yellow 153 [C.I. 48545, (CAS: 29204-84-0)], Pigment Yellow 155 [C.I. 200310, (CAS: 68516-73-4)], Pigment Yellow 157 [C.I. 77900, (CAS: 68610-24-2)], Pigment Yellow 158 [C.I. 77862, (CAS: 68186-93-6)], Pigment Yellow 159 [C.I. 77997, (CAS: 68187-15-5)], Pigment Yellow 16 [C.I. 20040, (CAS: 5979-28-2)], Pigment Yellow 160 [C.I. 77991, (CAS: 68187-01-9)], Pigment Yellow 161 [C.I. 77895, (CAS: 68611-43-8)], Pigment Yellow 162 [C.I. 77896, (CAS: 68611-42-7)], Pigment Yellow 163 [C.I. 77897, (CAS: 68186-92-5)], Pigment Yellow 164 [C.I. 77899, (CAS: 68412-38-4)], Pigment Yellow 167 [C.I. 11737, (CAS: 38489-24-6)], Pigment Yellow 168 [C.I. 13960, (CAS: 71832-85-4)], Pigment Yellow 169 [C.I. 13955, (CAS: 73385-03-2)], Pigment Yellow 17 [C.I. 21105, (CAS: 4531-49-1)], Pigment Yellow 173 [C.I. 561600, (CAS: 51016-63-8)], Pigment Yellow 174 [C.I. 21098, (CAS: 78952-72-4)], Pigment Yellow 176 [C.I. 21103, (CAS: 90268-24-9)], Pigment Yellow 177 [C.I. 48120, (CAS: 60109-88-8)], Pigment Yellow 179 [C.I. 48125, (CAS: 63287-28-5)], Pigment Yellow 180 [C.I. 21290, (CAS: 77804-81-0)], Pigment Yellow 181 [C.I. 11777, (CAS: 74441-05-7)], Pigment Yellow 182 [C.I. 128300, (CAS: 67906-31-4)], Pigment Yellow 183 [C.I. 18792, (CAS: 65212-77-3)], Pigment Yellow 184 [C.I. 771740, (CAS: 14059-33-7)], Pigment Yellow 185 [C.I. 56290, (CAS: 76199-85-4)], Pigment Yellow 188 [C.I. 21094, (CAS: 23792-68-9)], Pigment Yellow 190 [C.I. 189785, (CAS: 94612-75-6)], Pigment Yellow 191 [C.I. 18795, (CAS: 129423-54-7)], Pigment Yellow 191 [C.I. 18795, (CAS: 154946-66-4)], Pigment Yellow 192 [C.I. 507300, (CAS: 56279-27-7)], Pigment Yellow 193 [C.I. 65412, (CAS: 70321-14-1)], Pigment Yellow 194 [C.I. 11785, (CAS: 82199-12-0)], Pigment Yellow 199 [C.I. 653200, (CAS: 136897-58-0)], Pigment Yellow 2 [C.I. 11730, (CAS: 6486-26-6)], Pigment Yellow 202 [C.I. 65410, (CAS: 3627-47-2)], Pigment Yellow 203 [C.I. 117390, (CAS: 150959-17-4)], Pigment Yellow 213 [C.I. 117875, (CAS: 220198-21-0)], Pigment Yellow 218 [C.I. 561805, (CAS: 910868-14-3)], Pigment Yellow 220 [C.I. 561806, (CAS: 17352-39-5)], Pigment Yellow 227 [C.I. 777895, (CAS: 1374645-21-2)], Pigment Yellow 3 [C.I. 11710, (CAS: 6486-23-3)], Pigment Yellow 30 [C.I. 77592, (CAS: 1345-30-8)], Pigment Yellow 31 [C.I. 77103, (CAS: 10294-40-3)], Pigment Yellow 33 [C.I. 77223, (CAS: 8012-75-7)], Pigment Yellow 34 [C.I. 77603, (CAS: 1344-37-2)], Pigment Yellow 35 [C.I. 77205, (CAS: 90604-89-0)], Pigment Yellow 36 [C.I. 77956, (CAS: 49663-84-5)], Pigment Yellow 37 [C.I. 77199, (CAS: 90604-90-3)], Pigment Yellow 38 [C.I. 77878, (CAS: 1315-01-1)], Pigment Yellow 39 [C.I. 77086, (CAS: 1303-33-9)], Pigment Yellow 4 [C.I. 11665, (CAS: 1657-16-5)], Pigment Yellow 41 [C.I. 77588, (CAS: 8012-00-8)], Pigment Yellow 42 [C.I. 77492, (CAS: 51274-00-1)], Pigment Yellow 43 [C.I. 77492, (CAS: 64294-91-3)], Pigment Yellow 44 [C.I. 77188, (CAS: 1345-08-0)], Pigment Yellow 45 [C.I. 77505, (CAS: 1328-64-9)], Pigment Yellow 46 [C.I. 77577, (CAS: 1317-36-8)], Pigment Yellow 48 [C.I. 77610, (CAS: 592-05-2)], Pigment Yellow 5 [C.I. 11660, (CAS: 4106-67-6)], Pigment Yellow 53 [C.I. 77788, (CAS: 8007-18-9)], Pigment Yellow 55 [C.I. 21096, (CAS: 6358-37-8)], Pigment Yellow 6 [C.I. 11670, (CAS: 4106-76-7)], Pigment Yellow 60 [C.I. 12705, (CAS: 6407-74-5)], Pigment Yellow 61 [C.I. 13880, (CAS: 5280-69-3)], Pigment Yellow 62 [C.I. 13940, (CAS: 12286-66-7)], Pigment Yellow 62 [C.I. 13940, (CAS: 5280-70-6)], Pigment Yellow 65 [C.I. 11740, (CAS: 6528-34-3)], Pigment Yellow 7 [C.I. 12780, (CAS: 6407-81-4)], Pigment Yellow 73 [C.I. 11738, (CAS: 13515-40-7)], Pigment Yellow 74 [C.I. 11741, (CAS: 6358-31-2)], Pigment Yellow 75 [C.I. 11770, (CAS: 52320-66-8)], Pigment Yellow 77 [C.I. 20045, (CAS: 5905-17-9)], Pigment Yellow 81 [C.I. 21127, (CAS: 22094-93-5)], Pigment Yellow 83 [C.I. 21108, (CAS: 5567-15-7)], Pigment Yellow 83 [C.I. 21107, (CAS: 15110-84-6)], Pigment Yellow 9 [C.I. 11720, (CAS: 6486-24-4)], Pigment Yellow 93 [C.I. 20710, (CAS: 5580-57-4)], Pigment Yellow 94 [C.I. 20038, (CAS: 5580-58-5)], Pigment Yellow 95 [C.I. 20034, (CAS: 5280-80-8)], Pigment Yellow 98 [C.I. 11727, (CAS: 32432-45-4)], Prussian blue [C.I. 77510, (CAS: 12240-15-2)], Pigment Blue 1 [(CAS: 1325-87-7)], Pigment Blue 1 [(CAS: 69980-72-9)], Pigment Blue 1 [(CAS: 68409-66-5)], Pigment Blue 10 [(CAS: 84057-86-3)], Pigment Blue 12 [(CAS: 1325-77-5)], Pigment Blue 14 [(CAS: 1325-88-8)], Pigment Blue 2 [(CAS: 1325-94-6)], Pigment Blue 3 [(CAS: 1325-79-7)], Pigment Blue 9 [(CAS: 1325-74-2)], Pigment Green 1 [(CAS: 1325-75-3)], Pigment Green 3 [(CAS: 68845-37-4)], Pigment Green 4 [(CAS: 61725-50-6)], Pigment Red 80 [(CAS: 12224-98-5)], Pigment Red 81 [(CAS: 80083-40-5)], Pigment Red 81 [(CAS: 75627-12-2)], Pigment Red 81 [(CAS: 68310-07-6)], Pigment Red 81 [(CAS: 85959-61-1)], Pigment Red 81 [(CAS: 63022-06-0)], Pigment Red 81 [(CAS: 63022-07-1)], Pigment Violet 1 [(CAS: 1326-03-0)], Pigment Violet 2 [(CAS: 1326-04-1)], Pigment Violet 2 [(CAS: 103443-41-0)], Pigment Violet 4 [(CAS: 1325-80-0)], Pigment Black 1 [(CAS: 73104-73-1)], Pigment Black 1 [(CAS: 9064-44-2)], Pigment Black 11 [(CAS: 120899-48-1)], Pigment Black 11 [(CAS: 128666-38-6)], Pigment Black 11 [(CAS: 128666-37-5)], Pigment Black 11 [(CAS: 128666-36-4)], Pigment Black 11 [(CAS: 147858-25-1)], Pigment Black 16 [(CAS: 7440-66-6)], Pigment Black 19 [(CAS: 874954-47-9)], Pigment Black 2 [(CAS: 12236-57-6)], Pigment Black 20 [(CAS: 12216-93-2)], Pigment Black 21 [(CAS: 12216-94-3)], Pigment Black 22 [(CAS: 55353-02-1)], Pigment Black 3 [(CAS: 945563-42-8)], Pigment Black 35 [(CAS: 945563-51-9)], Pigment Black 5 [(CAS: 945563-45-1)], Pigment Blue 1 [(CAS: 68647-33-6)], Pigment Blue 10 [(CAS: 308086-15-9)], Pigment Blue 11 [(CAS: 71798-70-4)], Pigment Blue 13 [(CAS: 945558-73-6)], Pigment Blue 15-Pigment Green 7 mixt. [(CAS: 1026025-11-5)], Pigment Blue 15-Pigment Red 122-Pigment Yellow 74 mixt. [(CAS: 1357447-02-9)], Pigment Blue 151 [(CAS: 685529-31-1)], Pigment Blue 16 [(CAS: 424827-05-4)], Pigment Blue 17 [(CAS: 153640-87-0)], Pigment Blue 17 [(CAS: 71799-04-7)], Pigment Blue 19 [(CAS: 58569-23-6)], Pigment Blue 2 [(CAS: 1126074-38-1)], Pigment Blue 20 [(CAS: 945558-74-7)], Pigment Blue 209 [(CAS: 215590-82-2)], Pigment Blue 23 [(CAS: 57486-30-3)], Pigment Blue 24 [(CAS: 1042940-03-3)], Pigment Blue 28 [(CAS: 151732-17-1)], Pigment Blue 29 [(CAS: 151732-19-3)], Pigment Blue 31 [(CAS: 945558-75-8)], Pigment Blue 4 [(CAS: 945558-70-3)], Pigment Blue 5 [(CAS: 945558-72-5)], Pigment Blue 52 [(CAS: 945558-90-7)], Pigment Blue 53 [(CAS: 945558-91-8)], Pigment Blue 53 [(CAS: 190454-42-3)], Pigment Blue 56 [(CAS: 64427-27-6)], Pigment Blue 58 [(CAS: 12236-58-7)], Pigment Blue 59 [(CAS: 12236-59-8)], Pigment Blue 6 [(CAS: 371759-37-4)], Pigment Blue 61 [(CAS: 1126075-97-5)], Pigment Blue 63 [(CAS: 815586-00-6)], Pigment Blue 67 [(CAS: 945558-93-0)], Pigment Blue 68 [(CAS: 129406-28-6)], Pigment Blue 69 [(CAS: 945558-94-1)], Pigment Blue 7 [(CAS: 71838-91-0)], Pigment Blue 7 [(CAS: 120177-75-5)], Pigment Blue 70 [(CAS: 72827-99-7)], Pigment Blue 77 [(CAS: 945558-95-2)], Pigment Blue 8 [(CAS: 12224-90-7)], Pigment Blue 80 [(CAS: 391663-82-4)], Pigment Blue 81 [(CAS: 945558-98-5)], Pigment Blue 83 [(CAS: 1126076-49-0)], Pigment Blue 84 [(CAS: 2095508-48-6)], Pigment Brown 126 [(CAS: 128664-60-8)], Pigment Brown 29 [(CAS: 109414-04-2)], Pigment Brown 3 [(CAS: 1325-24-2)], Pigment Brown 30 [(CAS: 135668-57-4)], Pigment Brown 31 [(CAS: 126338-71-4)], Pigment Brown 32 [(CAS: 72828-00-3)], Pigment Brown 36 [(CAS: 945563-08-6)], Pigment Brown 4 [(CAS: 109944-91-4)], Pigment Brown 40 [(CAS: 945563-13-3)], Pigment Brown 41 [(CAS: 211502-16-8)], Pigment Brown 42 [(CAS: 211502-17-9)], Pigment Brown 43 [(CAS: 75864-23-2)], Pigment Brown 44 [(CAS: 945563-18-8)], Pigment Brown 45 [(CAS: 945563-37-1)], Pigment Brown 46 [(CAS: 945563-38-2)], Pigment Brown 47 [(CAS: 945563-39-3)], Pigment Brown 48 [(CAS: 2170864-80-7)], Pigment Brown 5 [(CAS: 16521-34-9)], Pigment Brown 6 [(CAS: 1275574-14-5)], Pigment Green 1 [(CAS: 68814-00-6)], Pigment Green 1 [(CAS: 68123-12-6)], Pigment Green 13 [(CAS: 148092-61-9)], Pigment Green 14 [(CAS: 114013-40-0)], Pigment Green 16 [(CAS: 65505-26-2)], Pigment Green 2 [(CAS: 12213-69-3)], Pigment Green 2 [(CAS: 76963-33-2)], Pigment Green 25 [(CAS: 945560-75-8)], Pigment Green 45 [(CAS: 945561-39-7)], Pigment Green 46 [(CAS: 945561-40-0)], Pigment Green 48 [(CAS: 945561-55-7)], Pigment Green 49 [(CAS: 945561-56-8)], Pigment Green 52 [(CAS: 945562-08-3)], Pigment Green 55 [(CAS: 945563-02-0)], Pigment Green 56 [(CAS: 945563-05-3)], Pigment Green 59 [(CAS: 2170445-83-5)], Pigment Green 6 [(CAS: 945559-56-8)], Pigment Green 62 [(CAS: 2108056-55-7)], Pigment Green 63 [(CAS: 2108056-56-8)], Pigment Green 7 [(CAS: 68022-83-3)], Pigment Green 77 [(CAS: 12715-62-7)], Pigment Green 7-Pigment Yellow 93 mixt. [(CAS: 1046461-83-9)], Pigment Orange 12 [(CAS: 945426-49-3)], Pigment Orange 20 [(CAS: 957128-28-8)], Pigment Orange 25 [(CAS: 12224-97-4)], Pigment Orange 32 [(CAS: 945426-51-7)], Pigment Orange 36 [(CAS: 12236-62-3)], Pigment Orange 38 [(CAS: 12236-64-5)], Pigment Orange 42 [(CAS: 12768-99-9)], Pigment Orange 43-Pigment Orange 64 mixt. [(CAS: 1046461-84-0)], Pigment Orange 47 [(CAS: 71819-73-3)], Pigment Orange 48 [(CAS: 71819-74-4)], Pigment Orange 49 [(CAS: 71819-75-5)], Pigment Orange 50 [(CAS: 76780-89-7)], Pigment Orange 51 [(CAS: 61512-61-6)], Pigment Orange 52 [(CAS: 61512-62-7)], Pigment Orange 53 [(CAS: 945426-52-8)], Pigment Orange 54 [(CAS: 945426-53-9)], Pigment Orange 55 [(CAS: 304891-88-1)], Pigment Orange 56 [(CAS: 74433-73-1)], Pigment Orange 57 [(CAS: 945426-54-0)], Pigment Orange 58 [(CAS: 945426-55-1)], Pigment Orange 59 [(CAS: 304891-93-8)], Pigment Orange 60 [(CAS: 68399-99-5)], Pigment Orange 62 [(CAS: 52846-56-7)], Pigment Orange 63 [(CAS: 76233-79-9)], Pigment Orange 70 [(CAS: 914936-31-5)], Pigment Orange 74 [(CAS: 516493-26-8)], Pigment Orange 76 [(CAS: 945426-61-9)], Pigment Orange 79 [(CAS: 945426-62-0)], Pigment Orange 8 [(CAS: 945426-48-2)], Pigment Orange 80 [(CAS: 945426-63-1)], Pigment Orange 81 [(CAS: 656223-72-2)], Pigment Orange 82 [(CAS: 2170864-77-2)], Pigment Orange 86 [(CAS: 1883421-38-2)], Pigment Orange 9 [(CAS: 71799-05-8)], Pigment Red 1 [(CAS: 39781-24-3)], Pigment Red 102 [(CAS: 1332-25-8)], Pigment Red 108 [(CAS: 918496-78-3)], Pigment Red 110 [(CAS: 854102-21-9)], Pigment Red 111 [(CAS: 12224-99-6)], Pigment Red 118 [(CAS: 945428-13-7)], Pigment Red 120 [(CAS: 57485-96-8)], Pigment Red 123 [(CAS: 24108-89-2)], Pigment Red 134 [(CAS: 12286-59-8)], Pigment Red 135 [(CAS: 945428-14-8)], Pigment Red 136 [(CAS: 945428-21-7)], Pigment Red 137 [(CAS: 71799-07-0)], Pigment Red 139 [(CAS: 12262-44-1)], Pigment Red 140 [(CAS: 383890-12-8)], Pigment Red 142 [(CAS: 109944-97-0)], Pigment Red 143 [(CAS: 12286-63-4)], Pigment Red 152 [(CAS: 405113-25-9)], Pigment Red 154 [(CAS: 109944-98-1)], Pigment Red 155 [(CAS: 109944-99-2)], Pigment Red 156 [(CAS: 109945-00-8)], Pigment Red 158 [(CAS: 945552-90-9)], Pigment Red 159 [(CAS: 109945-01-9)], Pigment Red 160 [(CAS: 854524-60-0)], Pigment Red 161 [(CAS: 945552-91-0)], Pigment Red 165 [(CAS: 12225-03-5)], Pigment Red 167 [(CAS: 12236-66-7)], Pigment Red 176 [(CAS: 12225-06-8)], Pigment Red 178 [(CAS: 3049-71-6)], Pigment Red 17-Pigment Red 150-Pigment White 18 mixt. [(CAS: 2247196-29-6)], Pigment Red 180 [(CAS: 12769-00-5)], Pigment Red 182 [(CAS: 61036-51-9)], Pigment Red 183 [(CAS: 51920-11-7)], Pigment Red 191 [(CAS: 85068-75-3)], Pigment Red 199 [(CAS: 61901-78-8)], Pigment Red 20 [(CAS: 945426-74-4)], Pigment Red 200 [(CAS: 67801-10-9)], Pigment Red 201 [(CAS: 68258-66-2)], Pigment Red 202-Pigment Violet 19 mixt. [(CAS: 1122063-75-5)], Pigment Red 203 [(CAS: 945553-87-7)], Pigment Red 204 [(CAS: 438231-79-9)], Pigment Red 205 [(CAS: 741692-71-7)], Pigment Red 206 [(CAS: 71819-76-6)], Pigment Red 207 [(CAS: 71819-77-7)], Pigment Red 215 [(CAS: 304892-29-3)], Pigment Red 217 [(CAS: 155421-17-3)], Pigment Red 218 [(CAS: 383891-32-5)], Pigment Red 219 [(CAS: 909006-21-9)], Pigment Red 223 [(CAS: 26789-26-4)], Pigment Red 225 [(CAS: 125270-32-8)], Pigment Red 227 [(CAS: 71872-64-5)], Pigment Red 228 [(CAS: 304898-64-4)], Pigment Red 234 [(CAS: 945554-26-7)], Pigment Red 237 [(CAS: 220424-27-1)], Pigment Red 238 [(CAS: 140114-63-2)], Pigment Red 239 [(CAS: 220424-28-2)], Pigment Red 240 [(CAS: 141489-67-0)], Pigment Red 241 [(CAS: 945554-27-8)], Pigment Red 244 [(CAS: 882858-66-4)], Pigment Red 245 [(CAS: 68016-05-7)], Pigment Red 246 [(CAS: 431991-59-2)], Pigment Red 249 [(CAS: 97955-62-9)], Pigment Red 25 [(CAS: 945426-75-5)], Pigment Red 250 [(CAS: 146358-78-3)], Pigment Red 252 [(CAS: 945554-31-4)], Pigment Red 26 [(CAS: 109944-92-5)], Pigment Red 262 [(CAS: 211502-19-1)], Pigment Red 263 [(CAS: 278792-06-6)], Pigment Red 270 [(CAS: 251086-13-2)], Pigment Red 272 [(CAS: 350249-32-0)], Pigment Red 276 [(CAS: 945554-32-5)], Pigment Red 277 [(CAS: 945554-33-6)], Pigment Red 278 [(CAS: 945554-34-7)], Pigment Red 279 [(CAS: 832743-59-6)], Pigment Red 280 [(CAS: 945554-58-5)], Pigment Red 281 [(CAS: 945554-64-3)], Pigment Red 282 [(CAS: 938065-79-3)], Pigment Red 283 [(CAS: 945554-67-6)], Pigment Red 284 [(CAS: 1089180-60-8)], Pigment Red 285 [(CAS: 1248412-35-2)], Pigment Red 29 [(CAS: 109944-93-6)], Pigment Red 34 [(CAS: 71872-60-1)], Pigment Red 35 [(CAS: 104491-86-3)], Pigment Red 46 [(CAS: 945427-33-8)], Pigment Red 47 [(CAS: 945427-55-4)], Pigment Red 48 [(CAS: 16013-44-8)], Pigment Red 48 [(CAS: 17797-35-2)], Pigment Red 48-Pigment Red 122 mixt. [(CAS: 1046461-81-7)], Pigment Red 48 [(CAS: 218138-44-4)], Pigment Red 48 [(CAS: 218138-41-1)], Pigment Red 48 [(CAS: 68023-17-6)], Pigment Red 51 [(CAS: 25705-30-0)], Pigment Red 51 [(CAS: 446242-29-1)], Pigment Red 52 [(CAS: 27757-95-5)], Pigment Red 52 [(CAS: 67828-72-2)], Pigment Red 52 [(CAS: 218138-27-3)], Pigment Red 53 [(CAS: 15958-19-7)], Pigment Red 56 [(CAS: 25310-96-7)], Pigment Red 57 [(CAS: 88593-07-1)], Pigment Red 58 [(CAS: 25310-97-8)], Pigment Red 59 [(CAS: 945427-99-6)], Pigment Red 60 [(CAS: 446245-60-9)], Pigment Red 63 [(CAS: 5858-84-4)], Pigment Red 63 [(CAS: 16510-21-7)], Pigment Red 63 [(CAS: 1325-13-9)], Pigment Red 64 [(CAS: 5858-77-5)], Pigment Red 68 [(CAS: 25311-19-7)], Pigment Red 71 [(CAS: 384329-78-6)], Pigment Red 72 [(CAS: 945428-03-5)], Pigment Red 73 [(CAS: 109944-94-7)], Pigment Red 74 [(CAS: 109944-95-8)], Pigment Red 75 [(CAS: 109944-96-9)], Pigment Red 78 [(CAS: 71799-06-9)], Pigment Red 81-Pigment White 21 mixt. [(CAS: 192390-71-9)], Pigment Red 82 [(CAS: 110927-51-0)], Pigment Red 88 [(CAS: 14295-43-3)], Pigment Red 90 [(CAS: 51868-24-7)], Pigment Red 92 [(CAS: 909006-04-8)], Pigment Red 94 [(CAS: 12213-62-6)], Pigment Red 96 [(CAS: 945428-04-6)], Pigment Red 97 [(CAS: 239795-92-7)], Pigment Red 98 [(CAS: 945428-07-9)], Pigment Violet 1 [(CAS: 63022-09-3)], Pigment Violet 1 [(CAS: 62973-79-9)], Pigment Violet 11 [(CAS: 875014-31-6)], Pigment Violet 11 [(CAS: 765310-46-1)], Pigment Violet 122 [(CAS: 104491-87-4)], Pigment Violet 123 [(CAS: 80619-33-6)], Pigment Violet 17 [(CAS: 945554-69-8)], Pigment Violet 18 [(CAS: 945554-81-4)], Pigment Violet 21 [(CAS: 945555-53-3)], Pigment Violet 26 [(CAS: 945556-80-9)], Pigment Violet 28 [(CAS: 12236-70-3)], Pigment Violet 30 [(CAS: 12225-07-9)], Pigment Violet 32 [(CAS: 12225-08-0)], Pigment Violet 34 [(CAS: 12612-32-7)], Pigment Violet 35 [(CAS: 55177-94-1)], Pigment Violet 39 [(CAS: 64070-98-0)], Pigment Violet 39 [(CAS: 68477-21-4)], Pigment Violet 4 [(CAS: 68310-88-3)], Pigment Violet 40 [(CAS: 61968-83-0)], Pigment Violet 41 [(CAS: 945557-07-3)], Pigment Violet 42 [(CAS: 71819-79-9)], Pigment Violet 43 [(CAS: 79665-29-5)], Pigment Violet 44 [(CAS: 87209-55-0)], Pigment Violet 45 [(CAS: 945557-40-4)], Pigment Violet 46 [(CAS: 945557-42-6)], Pigment Violet 5 [(CAS: 22297-70-7)], Pigment Violet 50 [(CAS: 76233-81-3)], Pigment Violet 51 [(CAS: 945557-43-7)], Pigment Violet 52 [(CAS: 945557-99-3)], Pigment Violet 53 [(CAS: 945558-15-6)], Pigment Violet 54 [(CAS: 1126076-80-9)], Pigment Violet 55 [(CAS: 1126076-86-5)], Pigment Violet 56 [(CAS: 1126076-93-4)], Pigment Violet 7 [(CAS: 16035-60-2)], Pigment Violet 9 [(CAS: 945554-68-7)], Pigment Yellow 1 [(CAS: 12240-03-8)], Pigment Yellow 102 [(CAS: 12236-74-7)], Pigment Yellow 103 [(CAS: 12225-22-8)], Pigment Yellow 106 [(CAS: 12225-23-9)], Pigment Yellow 107 [(CAS: 12270-64-3)], Pigment Yellow 113 [(CAS: 14359-20-7)], Pigment Yellow 120 [(CAS: 29920-31-8)], Pigment Yellow 121 [(CAS: 14569-54-1)], Pigment Yellow 122 [(CAS: 852620-87-2)], Pigment Yellow 125 [(CAS: 304891-45-0)], Pigment Yellow 131 [(CAS: 945423-41-6)], Pigment Yellow 132 [(CAS: 945424-04-4)], Pigment Yellow 135 [(CAS: 945424-77-1)], Pigment Yellow 136 [(CAS: 181285-33-6)], Pigment Yellow 140 [(CAS: 945425-58-1)], Pigment Yellow 141 [(CAS: 945425-59-2)], Pigment Yellow 142 [(CAS: 177020-91-6)], Pigment Yellow 143 [(CAS: 945425-60-5)], Pigment Yellow 144 [(CAS: 945425-61-6)], Pigment Yellow 145 [(CAS: 115742-72-8)], Pigment Yellow 146 [(CAS: 945425-66-1)], Pigment Yellow 149 [(CAS: 945425-67-2)], Pigment Yellow 150 [(CAS: 939382-97-5)], Pigment Yellow 151 [(CAS: 31837-42-0)], Pigment Yellow 154 [(CAS: 68134-22-5)], Pigment Yellow 156 [(CAS: 63661-26-7)], Pigment Yellow 165 [(CAS: 865763-85-5)], Pigment Yellow 166 [(CAS: 76233-82-4)], Pigment Yellow 170 [(CAS: 31775-16-3)], Pigment Yellow 171 [(CAS: 53815-04-6)], Pigment Yellow 172 [(CAS: 76233-80-2)], Pigment Yellow 175 [(CAS: 35636-63-6)], Pigment Yellow 178 [(CAS: 945425-73-0)], Pigment Yellow 17 [(CAS: 221358-38-9)], Pigment Yellow 18 [(CAS: 1326-11-0)], Pigment Yellow 18 [(CAS: 68310-89-4)], Pigment Yellow 186 [(CAS: 945425-92-3)], Pigment Yellow 187 [(CAS: 131439-24-2)], Pigment Yellow 189 [(CAS: 69011-05-8)], Pigment Yellow 191 [(CAS: 1051932-58-1)], Pigment Yellow 195 [(CAS: 135668-58-5)], Pigment Yellow 196 [(CAS: 945425-96-7)], Pigment Yellow 197 [(CAS: 945425-97-8)], Pigment Yellow 198 [(CAS: 516493-10-0)], Pigment Yellow 20 [(CAS: 61512-63-8)], Pigment Yellow 200 [(CAS: 945425-98-9)], Pigment Yellow 201 [(CAS: 945425-99-0)], Pigment Yellow 204 [(CAS: 945426-05-1)], Pigment Yellow 205 [(CAS: 945426-18-6)], Pigment Yellow 206 [(CAS: 945426-19-7)], Pigment Yellow 207 [(CAS: 945426-23-3)], Pigment Yellow 208 [(CAS: 945426-25-5)], Pigment Yellow 209 [(CAS: 945426-27-7)], Pigment Yellow 21 [(CAS: 945421-49-8)], Pigment Yellow 210 [(CAS: 945426-35-7)], Pigment Yellow 211 [(CAS: 945426-36-8)], Pigment Yellow 212 [(CAS: 945426-37-9)], Pigment Yellow 214 [(CAS: 577980-23-5)], Pigment Yellow 215 [(CAS: 913621-26-8)], Pigment Yellow 216 [(CAS: 817181-98-9)], Pigment Yellow 217 [(CAS: 945426-39-1)], Pigment Yellow 219 [(CAS: 874963-72-1)], Pigment Yellow 221 [(CAS: 945426-41-5)], Pigment Yellow 223 [(CAS: 2095507-47-2)], Pigment Yellow 224 [(CAS: 1207669-05-3)], Pigment Yellow 23 [(CAS: 4981-43-5)], Pigment Yellow 231 [(CAS: 2148300-50-7)], Pigment Yellow 25 [(CAS: 945421-63-6)], Pigment Yellow 26 [(CAS: 945421-64-7)], Pigment Yellow 27 [(CAS: 945421-65-8)], Pigment Yellow 28 [(CAS: 945421-66-9)], Pigment Yellow 29 [(CAS: 945421-67-0)], Pigment Yellow 34 [(CAS: 147858-25-1)], Pigment Yellow 36 [(CAS: 37300-23-5)], Pigment Yellow 37 [(CAS: 68859-25-6)], Pigment Yellow 40 [(CAS: 13782-01-9)], Pigment Yellow 47 [(CAS: 12060-00-3)], Pigment Yellow 50 [(CAS: 945421-71-6)], Pigment Yellow 51 [(CAS: 945421-76-1)], Pigment Yellow 56 [(CAS: 12225-09-1)], Pigment Yellow 58 [(CAS: 12225-11-5)], Pigment Yellow 61 [(CAS: 12286-65-6)], Pigment Yellow 72 [(CAS: 945421-81-8)], Pigment Yellow 79 [(CAS: 331414-25-6)], Pigment Yellow 8 [(CAS: 71872-65-6)], Pigment Yellow 80 [(CAS: 945421-85-2)], Pigment Yellow 82 [(CAS: 12225-14-8)], Pigment Yellow 84 [(CAS: 945421-87-4)], Pigment Yellow 85 [(CAS: 12286-67-8)], Pigment Yellow 86 [(CAS: 12286-68-9)], Pigment Yellow 86 [(CAS: 5280-65-9)], Pigment Yellow 88 [(CAS: 945422-67-3)], Pigment Yellow 89 [(CAS: 945422-85-5)], Pigment Yellow 90 [(CAS: 713104-87-1)], Pigment Yellow 91 [(CAS: 945423-18-7)], Pigment Yellow 96 [(CAS: 12213-63-7)], Pigment Yellow 97 [(CAS: 12225-18-2)], Pigment Yellow 99 [(CAS: 12225-20-6)]

The pigment(s) used in the color composition can include at least two different pigments selected from the above pigment group, or can include at least three different pigments selected from the above pigment group. According to an embodiment, the pigment(s) used in the color composition can include at least one yellow pigment selected from the yellow pigment group consisting of: a Pigment Yellow 83 (CI 21108), CAS #5567-15-7, Pigment Yellow 155 (C.I. 200310), (CAS: 68516-73-4), Pigment Yellow 180 (C.I. 21290), (CAS: 77804-81-0).

In addition to the at least one yellow pigment, or alternatively, the pigments(s) used in the color composition can include at least one red pigment selected from the red pigment group consisting of: Pigment Red 5 (CI 12490), (CAS #6410-41-9), Pigment Red 112 (CI 12370), (CAS #6535-46-2), Pigment Red 122 (CI 73915), (CAS #980-26-7).

In addition to the at least one yellow pigment and/or the at least one red pigment, or alternatively, the pigments(s) used in the color composition can include at least one green pigment selected from the green pigment group consisting of: Pigment Green 36, (C.I. 74265), (CAS: 14302-13-7).

In addition to the at least one yellow pigment and/or the at least one red pigment and or the at least one green pigment, or alternatively, the pigments(s) used in the color composition can include at least one blue pigment selected from the blue pigment group consisting of: Pigment Blue 16, (CAS: 424827-05-4), Pigment Blue 60 (C.I. 69800), (CAS: 81-77-6), Pigment Blue 66, (C.I. 73000), (CAS: 482-89-3)

In addition to the at least one yellow pigment and/or the at least one red pigment and/or the at least one green pigment, and/or the at least one blue pigment or alternatively, the pigments(s) used in the color composition can include at least one black pigment selected from the black pigment group consisting of: Pigment Black 6 (C.I. 77266), (CAS 1333-86-4), Pigment Black 7 (C.I. 77266), (CAS 1333-86-4).

The pigment(s) can optionally have a surface zeta potential of ≥+15 mV, preferably ≥±20 mV, more preferably ≥±25 mV. The surface zeta potential can be measured with a zetasizer, for example, a Zetasizer 3000 HS. Surface zeta potential measurements are conducted, for example, according to ISO 13099.

For example, the white or colored organic pigments can be chosen from carmine, carbon black, aniline black, melanin, azo yellow, quinacridone, phthalocyanin blue, sorghum red, the blue pigments codified in the Color Index under the references CI 42090, 69800, 69825, 73000, 74100, and 74160, the yellow pigments codified in the Color Index under the references CI 11680, 11710, 15985, 19140, 20040, 21090, 21100, 21108, 47000, 47005 and 77492.

The green pigments codified in the Color Index under the references CI 61565, 61570, 74265, and 74260, the orange pigments codified in the Color Index under the references CI 11725, 12075, 15510, 45370, and 71105, the red pigments codified in the Color Index under the references CI 12085, 12120, 12370, 12420, 12490, 14700, 15525, 15580, 15585, 15620, 15630, 15800, 15850, 15865, 15880, 17200, 26100, 45380, 45410, 45430, 58000, 73360, 73915, 75470, and 77491 and the pigments obtained by oxidative polymerization of indole or phenolic derivatives.

Non-limiting examples that can also be mentioned include pigmentary pastes of organic pigments, such as the products sold by the company Hoechst under the names: JAUNE COSMENYL IOG: Pigment Yellow 3 (CI 11710); JAUNE COSMENYL G: Pigment Yellow 1 (CI 11680); ORANGE COSMENYL GR: Pigment Orange 43 (CI 71105); ROUGE COSMENYL R: Pigment Red 4 (CI 12085); CARMINE COSMENYL FB: Pigment Red 5 (CI 12490); VIOLET COSMENYL RL: Pigment Violet 23 (CI 51319); BLEU COSMENYL A₂R: Pigment Blue 15.1 (CI 74160); VERT COSMENYL GG: Pigment Green 7 (CI 74260); and NOIR COSMENYL R: Pigment Black 7 (CI 77266).

The at least one pigment in accordance with the present disclosure can also be in the form of at least one composite pigment as described in European Patent Publication No. EP 1 184 426 A2. These composite pigments can be, for example, compounds of particles comprising a mineral core, at least one binder for ensuring the binding of the organic pigments to the core, and at least one organic pigment at least partially covering the core.

The at least one pigment in accordance with the present disclosure can be in the form of small undissolved microparticles, which do not diffuse into the hair color, but deposit on the outer wall of the keratin fiber. Suitable color pigments can be of organic and/or inorganic origin. But the pigments can also be inorganic color pigments, given the excellent light, weather and/or temperature resistance thereof.

Inorganic pigments, whether natural or synthetic in origin, include those produced from chalk, red ocher, umbra, green earth, burnt sienna or graphite, for example. Furthermore, it is possible to use black pigments, such as iron oxide black, color pigments such as ultramarine or iron oxide red, and fluorescent or phosphorescent pigments as inorganic color pigments.

Colored metal oxides, metal hydroxides and metal oxide hydrates, mixed phase pigments, sulfurous silicates, silicates, metal sulfides, complex metal cyanides, metal sulfates, metal chromates and/or metal molybdates are particularly suitable. In particular, preferred color pigments are black iron oxide (Cl 77499), yellow iron oxide (Cl 77492), red and brown iron oxide (Cl 77491), manganese violet (Cl 77742), ultramarine (sodium aluminum sulfosilicates, Cl 77007, Pigment Blue 29), chromium oxide hydrate (CI 77289), iron blue (ferric ferrocyanide, CI 77510) and/or carmine (cochineal).

The at least one pigment can also be colored pearlescent pigments. These are usually mica-based and can be coated with one or more metal oxides from the group consisting of titanium dioxide (CI 77891), black iron oxide (CI 77499), yellow iron oxide (CI 77492), red and brown iron oxide (Cl 77491, CI 77499), manganese violet (Cl 77742), ultramarine (sodium aluminum sulfosilicates, CI 77007, Pigment Blue 29), chromium oxide hydrate (CI 77289), chromium oxide (CI 77288) and/or iron blue (ferric ferrocyanide, CI 77510).

Mica forms part of the phyllosilicates, including muscovite, phlogopite, paragonite, biotite, lepidolite, and margarite. To produce the pearlescent pigments in combination with metal oxides, the mica, primarily muscovite or phlogopite, is coated with a metal oxide.

As an alternative to natural mica, it is also optionally possible to use synthetic mica coated with one or more metal oxides as the pearlescent pigment. Such suitable pearlescent pigments based on natural micas are described in, e.g., WO 2005/065632. The at least one pigment can also be pearlescent pigments based on natural or synthetic mica and are coated with one or more of the aforementioned metal oxides. The color of the respective pigments can be varied by varying the layer thickness of the metal oxide or metal oxides.

The at least one pigment can also be at least one inorganic color pigment selected from the group consisting of colored metal oxides, metal hydroxides, metal oxide hydrates, silicates, metal sulfides, complex metal cyanides, metal sulfates, bronze pigments and/or colored pigments based on mica, which are coated with at least one metal oxide and/or a metal oxychloride.

The at least one pigment can also be at least one mica-based colored pigment, which is coated with one or more metal oxides from the group consisting of titanium dioxide (CI 77891), black iron oxide (CI 77499), yellow iron oxide (CI 77492), red and/or brown iron oxide (Cl 77491, CI 77499), manganese violet (Cl 77742), ultramarine (sodium aluminum sulfosilicates, CI 77007, Pigment Blue 29), chromium oxide hydrate (CI 77289), chromium oxide (CI 77288) and/or iron blue (ferric ferrocyanide, CI 77510).

The at least one pigment can also be color pigments commercially available, for example, under the trade names Rona®, Colorona®, Dichrona® and Timiron® from Merck, Ariabel® and Unipure® from Sensient, Prestige® from Eckart Cosmetic Colors, and Sunshine® from Sunstar.

The at least one pigment can also be color pigments bearing the trade name Colorona® are, for example: Colorona Copper, Merck, MICA, Cl 77491 (IRON OXIDES); Colorona Passion Orange, Merck, Mica, Cl 77491 (Iron Oxides), Alumina; Colorona Patina Silver, Merck, MICA, Cl 77499 (IRON OXIDES), Cl 77891 (TITANIUM DIOXIDE); Colorona RY, Merck, Cl 77891 (TITANIUM DIOXIDE), MICA, Cl 75470 (CARMINE); Colorona Oriental Beige, Merck, MICA, Cl 77891 (TITANIUM DIOXIDE), Cl 77491 (IRON OXIDES); Colorona Dark Blue, Merck, MICA, TITANIUM DIOXIDE, FERRIC FERROCYANIDE; Colorona Chameleon, Merck, Cl 77491 (IRON OXIDES), MICA; Colorona Aborigine Amber, Merck, MICA, Cl 77499 (IRON OXIDES), Cl 77891 (TITANIUM DIOXIDE); Colorona Blackstar Blue, Merck, Cl 77499 (IRON OXIDES), MICA; Colorona Patagonian Purple, Merck, MICA, Cl 77491 (IRON OXIDES), Cl 77891 (TITANIUM DIOXIDE), Cl 77510 (FERRIC FERROCYANIDE); Colorona Red Brown, Merck, MICA, Cl 77491 (IRON OXIDES), Cl 77891 (TITANIUM DIOXIDE); Colorona Russet, Merck, Cl 77491 (TITANIUM DIOXIDE), MICA, Cl 77891 (IRON OXIDES); Colorona Imperial Red, Merck, MICA, TITANIUM DIOXIDE (Cl 77891), D&C RED NO. 30 (Cl 73360); Colorona Majestic Green, Merck, Cl 77891 (TITANIUM DIOXIDE), MICA, Cl 77288 (CHROMIUM OXIDE GREENS); Colorona Light Blue, Merck, MICA, TITANIUM DIOXIDE (Cl 77891), FERRIC FERROCYANIDE (Cl 77510); Colorona Red Gold, Merck, MICA, Cl 77891 (TITANIUM DIOXIDE), Cl 77491 (IRON); Colorona Gold Plus MP 25, Merck, MICA, TITANIUM DIOXIDE (Cl 77891), IRON OXIDES (Cl 77491); Colorona Carmine Red, Merck, MICA, TITANIUM DIOXIDE, CARMINE Colorona Blackstar Green, Merck, MICA, Cl 77499 (IRON OXIDES); Colorona Bordeaux, Merck, MICA, Cl 77491 (IRON OXIDES); Colorona Bronze, Merck, MICA, Cl 77491 (IRON OXIDES); Colorona Bronze Fine, Merck, MICA, Cl 77491 (IRON OXIDES); Colorona Fine Gold MP 20, Merck, MICA, Cl 77891 (TITANIUM DIOXIDE), Cl 77491 (IRON OXIDES); Colorona Sienna Fine, Merck, Cl 77491 (IRON OXIDES), MICA Colorona Sienna, Merck, MICA, Cl 77491 (IRON OXIDES); Colorona Precious Gold, Merck, Mica, Cl 77891 (Titanium dioxide), Silica, Cl 77491 (Iron oxides), Tin oxide; Colorona Sun Gold Sparkle MP 29, Merck, MICA, TITANIUM DIOXIDE, IRON OXIDES, MICA, Cl 77891, Cl 77491 (EU); Colorona Mica Black, Merck, Cl 77499 (Iron oxides), Mica, Cl 77891 (Titanium dioxide) Colorona Bright Gold, Merck, Mica, Cl 77891 (Titanium dioxide), Cl 77491 (Iron oxides); Colorona Blackstar Gold, Merck, MICA, Cl 77499 (IRON OXIDES); color pigments bearing the trade name Unipure® are, for example: Unipure Red LC 381 EM, Sensient Cl 77491 (Iron Oxides), Silica; Unipure Black LC 989 EM, Sensient, Cl 77499 (Iron Oxides), Silica; Unipure Yellow LC 182 EM, Sensient, Cl 77492 (Iron Oxides), Silica.

Depending on the degree of the change in color that is desired on the keratin fiber, the at least one pigment can also be can be used in varying amounts. The more color pigment that is used, the higher is the extent of the change in color in general. Starting at a certain usage amount, however, the adherence of the pigments to the keratin fiber approaches a limiting value, beyond which it is no longer possible to increase the extent of the change in color by further increasing the pigment amount used. While not wishing to be bound by any specific theory, it is believed that when a certain thickness is achieved, an insignificant amount of the incident lights passes through the pigment layer to make a difference to the observed color due to the hair itself. The rest of the light is either scattered back towards the surface or absorbed.

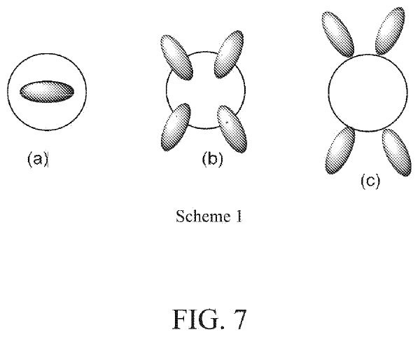

As illustrated in FIG. 7, the at least one pigment can be partially (Scheme 1, (b), where the dark oval represents a pigment, even though the pigment can be white or colorless) or completely enveloped in a matrix (e.g., a polymer matrix or an inorganic matrix; (Scheme 1, (a)). Or the pigment can be adhered to the surface of a matrix that can be colored or colorless (Scheme 1 (c)).

The matrix can be, e.g., CaCO₃, MnCO₃. Or the matrix can be a melamine formaldehyde matrix.

In another example, the at least one pigment can be encapsulated in silica, as described in Published U.S. Appl. No. 2007/0134180. Other examples of encapsulated pigments include encapsulated Carmine, Iron Oxides, Titanium dioxide, and Chrome Oxide/Hydroxide, the colorants D&C Red 21 Aluminum Lake, D&C Red 7 Calcium Lake, D&C Green 6 Liposoluble, and Aluminium Blue #1 (Indigo Carmine Lake). The encapsulated pigment can be titanium dioxide (used to lighten other pigments and to lend opacity to formulations) in any one of its mineral forms anatase, brookite or rutile, or mixtures thereof. Or the pigment can be at least one iron oxide in any of the 3 basic colors—red, black and yellow iron oxides, or mixtures thereof. From these 3 oxides and the addition of titanium dioxide, any shade of brown (skin tones) can be achieved.

The organic pigment can also be a lake. As used herein, the term “lake” means at least one dye adsorbed onto insoluble particles, the assembly thus obtained remaining insoluble during use. The inorganic substrates onto which the dyes are adsorbed can be, for example, alumina, silica, calcium sodium borosilicate, calcium aluminum borosilicate, calcium carbonate, manganese carbonate, aluminum, nitro-dyes, triarylmethin dyes, Azo-dyes, Anthrazen, Acid dyes, polymethine dyes, triarylmethin dyes, aza annulene dyes and polymethine dyes.

Among the dyes, non-limiting mention can be made of cochineal carmine. Non-limiting mention can also be made of the dyes known under the following names: D&C Red 21 (CI 45 380), D&C Orange 5 (CI 45 370), D&C Red 27 (CI 45 410), D&C Orange 10 (CI 45 425), D&C Red 3 (CI 45 430), D&C Red 4 (CI 15 510), D&C Red 33 (CI 17 200), D&C Yellow 5 (CI 19 140), D&C Yellow 6 (CI 15 985), D&C Green (CI 61 570), D&C Yellow 1 O (CI 77 002), D&C Green 3 (CI 42 053), and D&C Blue 1 (CI 42 090). A non-limiting example of a lake that can be mentioned is the product known under the following name: D&C Red 7 (CI 15 850:1).

The at least one pigment can also be a pigment with special effects. As used herein, the term “pigments with special effects” means pigments that generally create a non-uniform colored appearance (characterized by a certain shade, a certain vivacity, and a certain lightness) that changes as a function of the conditions of observation (light, temperature, observation angles, etc.). They thus contrast with white or colored pigments that afford a standard uniform opaque, semi-transparent, or transparent shade.

Several types of pigments with special effects exist, including those with a low refractive index, such as fluorescent, photochromic, or thermochromic pigments, and those with a high refractive index, such as nacres or glitter flakes. Examples of pigments with special effects of which non-limiting mention can be made include nacreous pigments such as mica coated with titanium or with bismuth oxychloride, colored nacreous pigments such as titanium mica with iron oxides, titanium mica for example with ferric blue or with chromium oxide, titanium mica with an organic pigment of the abovementioned type, and also nacreous pigments based on bismuth oxychloride. Nacreous pigments of which non-limiting mention can be made include the CELLINI nacres sold by Engelhard (mica-TiO₂-lake), PRESTIGE sold by Eckart (mica-TiO₂), PRESTIGE BRONZE sold by Eckart (mica-Fe₂O₃), and COLORONA sold by Merck (mica-TiO₂—Fe₂O₃).

In addition to nacres on a mica support, multilayer pigments based on synthetic substrates such as alumina, silica, sodium calcium borosilicate, calcium aluminum borosilicate, and aluminum, can be envisaged.

Non-limiting mention can also be made of pigments with an interference effect that are not fixed onto a substrate, for instance liquid crystals (HELICONES HC from Wacker) and holographic interference flakes (GEOMETRIC PIGMENTS or SPECTRA F/X from Spectratek). Pigments with special effects also comprise fluorescent pigments, whether these are substances that are fluorescent in daylight or that produce an ultraviolet fluorescence, phosphorescent pigments, photochromic pigments, thermochromic pigments, and quantum dots, sold, for example, by the company Quantum Dots Corporation.

Quantum dots are luminescent semiconductive nanoparticles capable of emitting, under light excitation, irradiation with a wavelength ranging from 400 nm to 700 nm. These nanoparticles are known from the literature. They can be manufactured, for example, according to the processes described, for example, in U.S. Pat. No. 6,225,198 or 5,990,479, in the publications cited therein, and also in the following publications: Dabboussi B. O. et al. “(CdSe)ZnS core-shell quantum dots: synthesis and characterization of a size series of highly luminescent nanocrystallites” Journal of Physical Chemistry B, vol. 101, 1997 pp. 9463-9475 and Peng, Xiaogang et al. “Epitaxial growth of highly luminescent CdSe/CdS core/shell nanocrystals with photostability and electronic accessibility”, Journal of the American Chemical Society, vol. 119, No. 30, pp. 7019-7029, all of the foregoing publications are incorporated herein by reference.

The variety of pigments that can be used in the present disclosure makes it possible to obtain a wide range of colors, and also optical effects such as metallic effects or interference effects.

The pigments that can be used in the present disclosure can transmit light of various wavelengths, including visible light (e.g., light having a wavelength of above 350 nm). The pigment(s) can also transmit light of certain wavelengths, but also reflect light of certain wavelengths. And the pigment(s) can also be 100% reflective. For examples, reflective pigments provide a high specular reflection of visible light. Reflective pigments include those that are partially or completely coated with a non-matt and non-scattering surface layer of a metal or metal oxide. The substrate can be chosen from glasses, ceramics, graphite, metal oxides, aluminas, silicas, silicates, especially aluminosilicates and borosilicates and synthetic mica (e.g., fluorophlogopite), to name a few. The metal or metal oxide can be, without limitation, titanium oxides, iron oxides, tin oxide, chromium oxide, barium sulfate, MgF₂, CeF₃, ZnS, ZnSe, SiO₂, Al₂O₃, MgO, Y₂O₃, SeO₃, SiO, HfO₂, ZrO₂, CeO₂, Nb₂O₅, Ta₂O₅ and MoS₂, and mixtures thereof. Reflective pigments can have a spectral reflectance in the visible spectrum of at least 70%.

Other reflective pigments include those having non-goniochromatic layered structure of two or more polymeric and/or metallic layers of different refractive indices. For example, reflective particles comprising layers of 2,6-polyethylene naphthalate (PEN) and of polymethyl (meth)acrylate are sold by 3M under the name Mirror Glitter™. Other effect pigments are available under the trade name Metasomes Standard/Glitter in various colors (yellow, red, green, blue) from Flora Tech.

### Color Gamut for Pigment Blends

CIE L*a*b* (CIELAB) is a color space specified by the International Commission on Illumination. It describes all the colors visible to the human eye and serves as a device-independent model to be used as a reference.

The three coordinates of CIELAB represent the lightness of the color (L*=0 yields black and L*=100 indicates diffuse white; specular white may be higher), its position between red/magenta and green (a*, negative values indicate green while positive values indicate magenta) and its position between yellow and blue (b*, negative values indicate blue and positive values indicate yellow).

Since the L*a*b* model is a three-dimensional model, it can be represented properly only in a three-dimensional space. Two-dimensional depictions include chromaticity diagrams: sections of the color solid with a fixed lightness.

Because the red-green and yellow-blue opponent channels are computed as differences of lightness transformations of (putative) cone responses, CIELAB is a chromatic value color space.

In the present invention, the color gamut is determined by adding each pigment to be tested in the hair coloring composition, and then individually tested at a level such that when applied to hair, the resulting CIELAB lightness or L* value of the colored hair is 60±2. The level of pigment needed will depend on the pigment being tested. Two hair tresses (Kerling, Natural White special quality) have the hair coloring composition applied as described in the present invention. A Minolta spectrophotometer CM-2600d is used to measure the color of the dried hair tresses, five points on both the front and back sides, and the values averaged. The D65 L*a*b values are calculated. When at least three pigments have each been measured such that their resulting color reside within the target L* values of 60±2 the color gamut can be calculated. First the lengths of each side of the resulting triangle of each combination of three pigments in the a*b plane are computed using the following expressions. To calculate the distance between pigments 1 and pigment 2 the following equation is used:

$$Side Length {SL}_{12} = {( {( {a}_{pigment 1} - {a}_{pigment 2} )}^{2} + {( {b}_{pigment 1} - {b}_{pigment 2} )}^{2} )}^{0.5} .$$

This is computed for each pair of pigments. Then for a series of three pigments.

The resulting color gamut is calculated using the expression:

$$Color Gamut = {( S ( S - S {L}_{12} ) ( S - S {L}_{13} ) ( S - S {L}_{23} ) )}^{0.5}$$

wherein SL₁₂, SL₁₃, and SL₂₃ are the three lengths of the sides of the triangle within the a*b plane, and S=(SL₁₂+SL₁₃+SL₂₃)/2. Where more than three pigments are used, this calculation can be performed for each combination of the three pigment from the more than three pigments used, and the largest Color Gamut is selected.

The hair coloring composition embodiments of the present invention can also have a color gamut of greater than 250, greater than 500, greater than 750, greater than 800, greater than 900, greater than 1100 or even greater than 1250.

### Experiments Performed for Color Gamut

Using the above expression, for each combination of three pigments possible from Color Gamut Tables 1, as illustrated below, the color gamut at a nominal L value of 60 was calculated.

Color Gamut Table 1

| | | | wt % | | | |
|---|---|---|---|---|---|---|
| Pigment | Name | Supplier | level | L | a | b |
| Blue 15 | PV Fast Blue BG-NIP | Clariant | 0.155 | 59.3 | −18.7 | −2.1 |
| Blue 16 | Phthalocyanine | Carbosynth | 0.280 | 59.4 | −17.3 | 1.5 |
| Blue 66 | Indigo 229296 | Aldrich | 0.105 | 60.0 | −3.1 | 6.8 |
| Blue 60 | Paliogen Blau L 6482 | BASF | 0.260 | 60.7 | −3.9 | 5.9 |
| Black 7 | Midnight Black | Geotech | 0.045 | 59.8 | 0.0 | 12.3 |
| Green 36 | Heliogen Green K 9362 | BASF | 0.509 | 60.1 | −32.8 | 20.2 |
| Red 112 | Permanent Red FGR 250 | Clariant | 0.150 | 60.1 | 29.8 | 18.8 |
| Red 122 | Hostaperm Pink E02-EDW | Clariant | 0.140 | 59.5 | 24.9 | 6.1 |
| | VP4034 | | | | | |
| Violet 19 | Ink Jet Magenta E5B 02 M250 | Clariant | 0.200 | 60.6 | 28.1 | 10.1 |
| Red 5 | Permanent Carmine FB01 | Clariant | 0.140 | 59.7 | 30.1 | 14.4 |
| Yellow 155 | Ink Jet Yellow 4GC | Clariant | 16.92 | 61.8 | 9.6 | 74.4 |
| Yellow 83 | Novoperm Yellow HR 70 | Clariant | 1.059 | 60.0 | 12.5 | 61.8 |
| Yellow 180 | Toner Yellow HG | Clariant | 9.16 | 61.4 | 11.2 | 72.8 |

These were formulated within an example formulation described later using an appropriate level of first, second and third compositions.

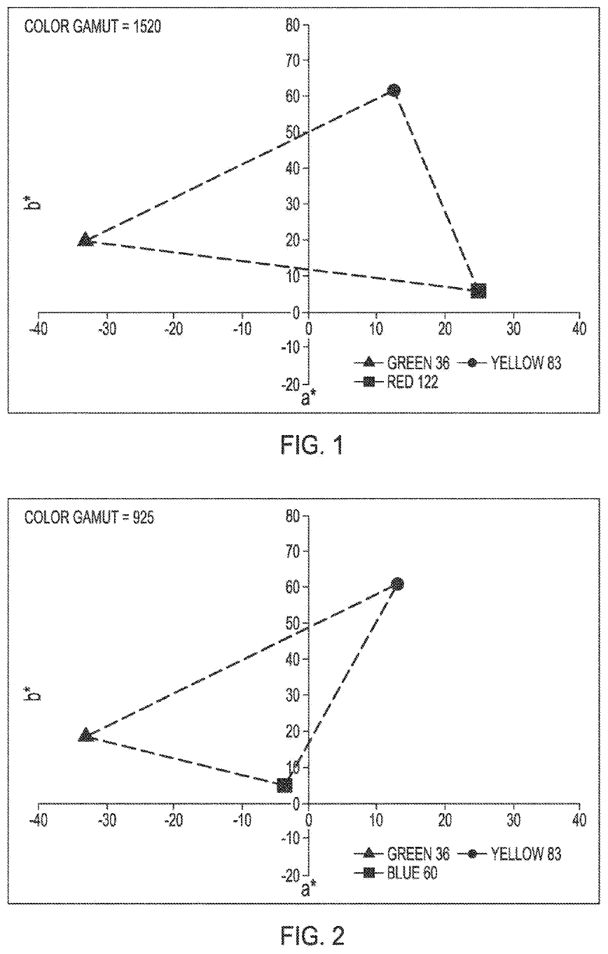

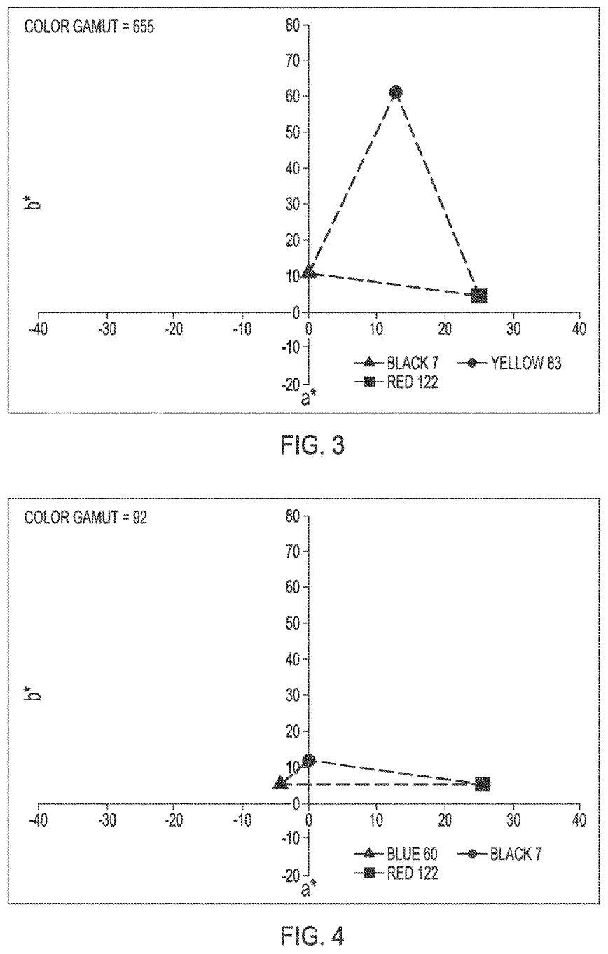

A few examples are exemplified of combinations of pigments and their resulting color gamut. One skilled in the art would be able to perform this for all of the possible permutations of pigments that are assessed according the description above.
FIGS. 1 to 6 show plots of color gamut triangles created for a series of three pigment selections.
FIG. 1 shows that a combination of Pigment Green 36, Pigment Yellow 83 and Pigment Red 122 a large triangle is plotted in the a*b* color plane with an area of 1520.
FIG. 2 shows that the combination of Pigment Green 36, Pigment Yellow 83 and Pigment Blue 60 gives a smaller triangle win an area of 925.
FIG. 3 shows the combination of Pigment Black 7, Pigment Yellow 83 and Pigment Red 122 gives a smaller triangle win an area of 655.
FIG. 4 shows the combination of Pigment Black 7, Pigment Blue 60 and Pigment Red 122 gives a smaller triangle win an area of 92.

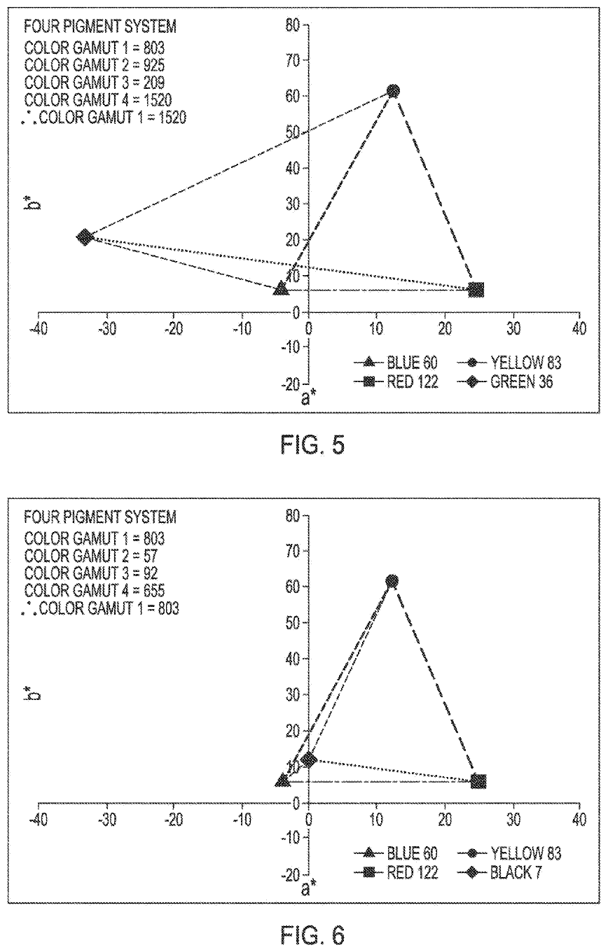

A second series of example are made for how to assess more than three pigments and their resulting color gamut. When plotted a series of triangles can be plotted as shown and for each the areas is assessed. For such a system the color gamut is defined as the largest of the triangles formed.
FIG. 5 shows a combination of Pigment Green 36, Pigment Yellow 83, Pigment Blue 60 and Pigment Red 122 a series of triangles are plotted with areas of 803, 925, 209 and 1520. The color gamut of this pigment system is 1520. [Alternative calculation of total area would yield, 1728]
FIG. 6 shows a combination of Pigment Black 7, Pigment Yellow 83, Pigment Blue 60 and Pigment Red 122 a series of triangles are plotted with areas of 803, 57, 92 and 655. The color gamut of the pigment system is 803 [alternative approach would be the same]

In an embodiment more than one multicomponent coloring composition can be applied to the hair in a sequential manner. For example it may be that a first multicomponent is applied to the hair which contains pigment microparticles that substantially scatter and/or reflect light such that it produces the visual effect of making the hair look lighter in color, after which a second multicomponent composition is applied which contains pigment microparticles that substantially absorbs light and provides color to the hair and wherein the combination of the sequential addition of a first and second multicomponent coloring compositions provides the final hair color. For example a first multicomponent coloring composition may comprise metallic flakes and the second multicomponent coloring composition may contain organic pigment microparticles. It may also be that more than a first a second multicomponent coloring are applied to the hair to achieve the desired color result, that three or more multicomponent coloring compositions are applied.

### THE pH

The multicomponent composition embodiments in accordance with the present disclosure can have a pH ranging from about 4 to about 10, preferably about 5 to about 9. The pH is also dynamically managed to control the rate of reaction of the reactive polymer components. Maintaining a slightly to moderately basic pH during the mixing and preapplication stages involving the first and second components controls the coupling reaction under certain circumstances. Maintaining a slightly acidic pH controls the coupling reaction under other certain circumstances. Depending upon the cationic or anionic capability of one of the linking reactants, the pH may be adjusted to minimize or negate its ability to link. For example, for carboxylic acids and carbodiimides, a basic pH will shift the acid-carbodiimide reaction toward the starting materials. For the Michael addition, an acidic pH will shift the amine-α,β olefinoyl reaction toward the starting materials. The linking may then be initiated by reversion of the pH to an appropriate state for reaction.

### Dispersants

It will be apparent to one skilled in the art that careful and selective choice of dispersant can help to maximize performance in terms of maximizing the amount of color produced from an immobilized film, maximizing the remanence or wash fastness, and enabling removal of the color.

For example, in the case where the binder polymer is anionic in nature, dispersants which are anionic or nonionic are preferably chosen, rather than cationic, as this avoids undesired precipitation in the formula prior to it forming a colored film on the keratin—i.e. utilizing the principle of avoiding opposing charges. The electrostatic, ionic and functional character of the dispersant also is chosen to be compatible with and to not interfere with the reactive polymer cross linking or with reactive termini. More preferably, the dispersant is chosen to be compatible with and miscible with the other components of the composition or compositions with and without medium. Under select circumstances, the dispersant may also be chosen not to be compatible with the pre-treatment composition if the pre-treatment composition is not formulated to include pigment. When these preferences are present, the dispersant migrates to the overlaid color composition layers of the coating and does not substantially mix with the pre-treatment coating layer. This arrangement can operate as a helpful adjunct during the removal step, the “off” step described below.

Likewise, the principle of choosing chemically similar dispersant with the polymers of the overlaid color compositions can be followed to ensure maximum compatibility.

As well as compatibility as noted above, the other critical criterion in selecting dispersant(s) is their ability to enable pigment to be dispersed down to the primary particle size, preferably with the minimum amount of input mechanical energy. It will be recognized by someone skilled in the art that the concentration of dispersing agent is also a critical factor. In general, it is usually required that there is a minimum amount for dispersing activity and that below this, the system is either not fully dispersed or, worse, that the dispersant acts as a flocculant.

These two considerations together are used to define preferred materials and their respective concentrations.

It may also be the case, depending on the type of binder polymer used, that the binder itself is also a dispersant. In such cases it is possible that no further dispersing additive may be needed.

Combination of the dispersed polymer mixture with the color composition or color compositions can be made in any manner and not with the pre-treatment composition. This order of combination of the dispersed polymer mixture with the color composition or color compositions delivers the dispersed pigment mixture with the color composition layers and on top of the pretreatment layer. While the layers intermix to a slight to moderate to essentially full extent, at least a portion of the dispersed pigment mixture resides over the pretreatment layer. This arrangement of the coating at least in part enables removal of the coating when the “off” techniques described below are practiced.

### Dispersants, Kinds, Properties and Chemistry

Dispersants are amphiphilic or amphiphathic meaning that they are chemical compounds possessing both hydrophilic (water-loving, polar) and lipophilic (fat-loving) properties. Dispersants are surface-active polymers that allow the homogeneous distribution and stabilization of solids, e.g. pigments in a liquid medium (like a binder), by lowering the interfacial tension between the two components. As a result, agglomerates are broken up into primary particles and protected by a protecting dispersant envelope of a re-agglomeration.

The dispersants can be subdivided on the basis of the stabilization mechanism in
1. dispersants for electrostatic stabilization
a. Anionic dispersing additives
i. Polyacrylates
ii. Polyphosphates
b. Neutral dispersing additives, e.g, nonionic surfactants
c. Cationic dispersing additives, e.g., quaternary ammonium organic and/or silicone polymers
2. Dispersants for steric stabilization

### Electrostatic Stabilization

The pigment surface is occupied by an additive carrying an ionic charge. All pigment particles are charged the same. The mutual repulsion by the charge is greater than the attractions of the pigment particles. The electrostatic stabilization has its relevance mostly in water-based paint systems.
Polyanionic dispersing additives: polycarboxylates (mostly salts of polyacrylic acids), polyphosphates divided into linear polyphosphates and cyclic metaphosphates, polyacrylates
salts of polyacrylic acid, as cations, sodium and ammonium are preferred, these polyacrylates are water-soluble, technical products have molecular weights in the range of 2000 to 20,000 g/mol, optimum is about 8000 g/mol
Sodium and ammonium salts of the homo- or copolymers of acrylic acid, methacrylic acid or maleic acid

### Steric Stabilization

The attractive forces between the pigment particles are effective only over relatively small distances of the particles from each other. The approach of two particles to each other can be prevented by molecules that are firmly anchored to the pigment surface and carry groups that extend from the surface and may reduce the potential for the pigments to contact one another. By sufficiently long chain lengths, agglomeration can be prevented. Also, the substances added to avoid agglomeration and other undesirable pigment particle interactions preferably are chosen to minimize or avoid interaction with the reactive polymers of the color composition.
Water-soluble polymers
Block or graft copolymers, so-called AB block copolymers
Example: AB block polymer of 2-vinylpyridine and methacrylic acid ester
Example: AB block copolymer of polyester (based caprolactam) and triethylenetetramine
Typical functional groups for the A segment are carboxyl, amine, sulfate and phosphate for inogenous bonds or polyether and polyamide for hydrogen bonds. B represents the solvated side chain, molecular weights 1000 to 15000 g/mol, e.g. modified polyacrylates or polyhydroxystearates; however, interference with the reactive polymers is to be minimized or preferably avoided.
Hydrophilic moieties (e.g., polyethers) and pigment affinic groups (e.g. Groups) containing oligomers or polymers.

The following types are distinguished according to the number of monomer types used in the production:
Homopolymers: only one kind of monomer
Copolymers: two monomers
Terpolymers: three monomers

Classification according to distribution of the monomers in the polymer:
Statistical polymers: A and B segments are distributed arbitrarily
Block polymers: the monomers are grouped into blocks
Graft polymers: these consist of a linear homopolymer backbone on which side chains of
other monomer blocks are grafted

Some examples of dispersants for solvent-based systems are:
oligomeric titanates and silanes for inorganic pigments with OH or carboxy groups.
Oligomeric polymeric carboxylic acids for inorganic pigments (cationic).
Polyamines for inorganic pigments, e.g., cationic polymers.
Salts of long-chain polyamines and polycarboxylic acids for inorganic and organic pigments (electroneutral).
Amine/amide-functional polyesters/polyacrylates for the stabilization of organic pigments.

Some examples of dispersants for aqueous systems are:
Inorganic dispersants such as fine-grained CaCO3, Ca3 (PO4) 2, polyphosphates, polyphosphoric acids.
Nonionic surfactants such as ethoxlyated fatty alcohol (e.g. Neodol 25-9), ethoxylated oils (e.g. ethxylated castor oil under the tradename Cremophore RH410)
Block and graft copolymers of the type having distinct hydrophilic and hydrophobic blocks (e.g. ethylene oxide-propylene oxide polymers under the tradename Poloxamer)
Anionic surfactants consisting of the unethoxylated or ethoxylated salts of acids (e.g. sodium ceteth-10-phosphate under the tradename Crodafos).

A detailed description and exemplary embodiments of the disperant component for pigments are set forth in detail in PCT application serial no PCT/EP2019/076647 filed Oct. 1, 2019, the disclosure of which is incorporated herein by reference.

### Incorporation of Pigment in Disperant

The pigments described herein can be chosen and/or modified to be similar enough such that a single dispersant can be used. In other instances, where the pigments are different, but compatible, two or more different dispersants can be used. Because of the extreme small size of the pigment microparticles and their affinity, combination of the pigment microparticles and dispersant to form a substantially homogeneous dispersion that can subsequently be modified and/or diluted as desired is to be accomplished before combination with any or all of the first, second and third components of the multicomponent composition and preferably with only the first and/or second components.

The pigment microparticles can be dispersed and stabilized in the medium by one or more dispersants the properties and kinds of which are described above. The dispersant can either be added to the medium, or to a precursor medium or can form a coating on the microparticles to facilitate dispersion. It is also possible to provide the microparticles with a coating of a dispersant material and additionally provide a further dispersant to the medium, or to a precursor medium, which is used to form the final medium.

The dispersant, either added to the medium or provided as coating, facilitates wetting of the microparticles, dispersing of the microparticles in the medium, and stabilizing of the microparticles in the medium.

The wetting includes replacing of materials, such as air, adsorbed on the surface of the pigment microparticles and inside of agglomerates of the microparticles by the medium. Typically, a complete wetting of the individual microparticles is desired to singularize the particles and to break off agglomerates formed by microparticles adhering to each other.

After wetting, the microparticles can be subjected to de-aggregate and de-agglomerate step, generally referred to as dispersing step. The dispersing step typically includes the impact of mechanical forces such as shear to singularize the microparticles. In addition to shearing to singularize, the microparticles can be broken into even smaller microparticles using, for example, roller mills, high speed mixers, and bead mills. Usual practice involves substantially homogeneous dispersion of the pigments in dispersant through the use of high shear mixing; for example, through use to the appropriate ball mill, ultra high-pressure homogenizer or other system known by those skilled in the art of pigment dispersion.

During wetting and dispersing, the exposed total surface area of the microparticles increases which is wetted by the dispersant. The amount of the dispersant may be gradually increased during dispersing to account for the increased surface area.

The dispersant also functions as de-flocculation agent keeping the dispersed microparticles in a dispersed state and prevent that they flocculate to form loose aggregates. This stabilization is also needed for long term storage purposes. Different type of stabilization such as electrostatic stabilization and steric stabilization are possible, and the type of dispersant is selected in view of the medium and the material of the microparticles.

The dispersant may be added to a dry powder of the pigment particles when the particles are milled to a desired size. During milling, or any other suitable technique to singularize the pigment particles or to break them into smaller part, the dispersant comes in contact with and adheres to the surface of the microparticles. Freshly generated microparticle surface during milling will be coated by the dispersant so that, after milling, the microparticles with a coating formed by the dispersant are provided.

The coating with the dispersant can also be carried out in a liquid carrier medium to which the dispersant is added. The microparticles can also be milled in the liquid carrier.

### Additive Components

Additive components of the composition include suspending agents, leveling agents and viscosity control agents. The suspending agents help maintain the pigment particles in dispersed condition and minimize or negate their agglomeration. Suspending agents include fatty acid esters of polyols such as polyethylene glycol and polypropylene glycol. These are similar to plasticizers and function in similar fashion to allow pigment particles to “slip” by each other without retarding or binding interaction. They act as grease in this fashion. Additionally, suspending agents in part participate in promoting the stable dispersion of the pigment particles and avoid settling. The polymers of the color composition also participate through their solubilization or interaction with the pigment particles and with the medium. The suspending agents provide another factor for maintaining the stable dispersion. They not only provide the “grease” to facilitate Brownian movement but also in part stabilize through interaction as emulsifiers of the pigment particles in the medium. Optional components also are to be chosen so that they do not interfere or only minimally interfere with the reactive polymer coupling reaction.

The color composition embodiments in accordance with the present invention can also optionally contain at least one adjuvant, chosen, for example, from reducing agents, fatty substances, softeners, antifoams, moisturizers, UV-screening agents, mineral colloids, peptizers, solubilizers, fragrances, anionic, cationic, nonionic, or amphoteric surfactants, proteins, vitamins, propellants, oxyethylenated or non-oxyethylenated waxes, paraffins, C₁₀-C₃₀ fatty acids such as stearic acid or lauric acid, and C₁₀-C₃₀ fatty amides such as lauric diethanolamide.

The multicomponent composition embodiments in accordance with the present invention can further optionally contain one or more additives, including, but not limited to, antioxidants (e.g., phenolics, secondary amines, phosphites, thioesters, and combinations thereof), crosslinking agents, reactive diluents (e.g., low molecular weight mono- or di-functional, non-aromatic, (meth)acrylate monomers such as 1,6-hexanediol di(meth)acrylate, tripropylene glycol di(meth)acrylate, isobornyl(meth)acrylate, 2(2-ethoxyethoxy)ethyl(meth)acrylate, n-vinyl formamide, tetrahydrofurfuryl(meth)acrylate, polyethylene glycol di(meth)acrylate, tripropylene glycol di(meth)acrylate, neopentyl glycol dialkoxy di(meth)acrylate, polyethyleneglycol di(meth)acrylate, and mixtures thereof), non-reactive diluents (e.g., ethylene glycol, di(ethylene glycol), tetra(ethylene glycol), glycerol, 1,5-pentanediol, ethylene glycol monomethyl ether, ethylene glycol dimethyl ether, triethylene glycol monomethyl ether, 2-ethoxyethanol, solketal, benzonitrile, hexamethylphosphoramide, 2-N-methylpyrrolidinone and N,N-dimethylformamide); dyes, fillers (e.g., silica; carbon black; clay; titanium dioxide; silicates of aluminum, magnesium, calcium, sodium, potassium and mixtures thereof, carbonates of calcium, magnesium and mixtures thereof; oxides of silicon, calcium, zinc, iron, titanium, and aluminum; sulfates of calcium, barium, and lead; alumina trihydrate; magnesium hydroxide and mixtures thereof), plasticizers (e.g., petroleum oils such as ASTM D2226 aromatic oils; paraffinic and naphthenic oils; polyalkylbenzene oils; organic acid monoesters such as alkyl and alkoxyalkyl oleates and stearates; organic acid diesters such as dialkyl, dialkoxyalkyl, and alkyl aryl phthalates, terephthalates, sebacates, adipates, and glutarates; glycol diesters such as tri-, tetra-, and polyethylene glycol dialkanoates; trialkyl trimellitates; trialkyl, trialkoxyalkyl, alkyl diaryl, and triaryl phosphates; chlorinated paraffin oils; coumarone-indene resins; pine tars; vegetable oils such as castor, tall, rapeseed, and soybean oils and esters and epoxidized derivatives thereof, esters of dibasic acids (or their anhydrides) with monohydric alcohols such as o-phthalates, adipates and benzoates; and the like and combinations thereof), processing aids, ultraviolet stabilizers (e.g., a hindered amine, an o-hydroxy-phenylbenzotriazole, a 2-hydroxy-4-alkoxybenzophenone, a salicylate, a cyanoacrylate, a nickel chelate, a benzylidene malonate, oxalanilide, and combinations thereof), and combinations thereof.

An additional additive may be a tactile hair modification agent. These may include, but are not limited to, a softening and/or lubricating and/or anti-static and/or hair alignment and/or anti-frizz benefit and/or impact on the keratin fibres.

Additional additives include filler materials such as but not limited to no chromatic material with a particle size of from about 2 nm to about 500 nm; macromolecular strands or nanoparticles composed of polyolefin such as polyethylene, polypropylene, polybutene, and combinations thereof, clays and mineralite substances such as but not limited to smectites, kaolins, illites, chlorites, attapulgites and intercalated aluminosilicate materials and purified formed thereof and combinations thereof. Additional mineral microparticles may be composed of inorganic metal oxides selected from the group consisting of silica, titanium oxide, zirconium oxide, aluminum oxide, magnesium oxide, boehmite alumina, hydrotalcite. Still other filler material includes but is not limited to carbon nanotubes micrographitic material such as nanofiller of graphite oxide mixed polymer, microbucky balls, clathrates, and crown composites of organic and mineral complexes. Additionally, the filler may be combined, complexed, contain or incorporate a polymer containing one of the members of a complementary reactive pair relating to the first and second components of the reactive polymer composition.

Additives may also include but are not limited to UV filter and UV block substances such as but not limited to avobenzone, bemotrizinol octocrylene, benzophenone-4, ethylhexyl methoxycinnamate, PABA, padimate O, PBSA, cinoxate, dioxybenzone, homosalate, menthyl anthranilate, octyl salicylate, parsol Max, tinosorb S and A2B, Uvinul, amioxate, polyvinylidene fluoride and other similar conjugated organic compounds, radical scavengers, triplet formation inhibitors, metal compounds incorporating chromium, titanium, zinc, nickel, manganese, iron, niobium, silver, gold, aluminum, hafnium, tantalum such as the oxides and similar forms thereof wherein the metal compounds absorb or reflect UV light.

### Topcoat

The topcoat composition is a post dressing composition that may be applied at a later time by the person whose hair has been dressed with the color coatings (hereinafter the user). The topcoat composition may also be applied to the user's hair by a salon professional who has previously dressed the user's hair with color coatings or who is in the process of dressing the user's hair. The topcoat typically contains a readily evaporable medium such as an aqueous alcohol mixture in combination with water repellant compounds, hair setting compounds that may be shampoo and/or water rinse removable, or hair setting compounds that may be slowly removable by cationic shampoo but not by water rinse or ordinary anionic shampoo typically applied as a home shampoo wash of hair. The topcoat composition may also be a preformed polymer composition as disclosed above as a color composition. As a topcoat composition, the preformed polymer composition may or may not contain pigment and preferably does not contain pigment.

In addition, the topcoat may further include a polymeric composition having one of the reactive functional groups of the reactive functional group complementary pair of the first and second components of the reactive polymer composition dressed on the user's hair to form a colored coating thereon. In this instance the topcoat may be dressed onto the user's hair by the salon professional who will adjust to concentration of corresponding first or second components of the reactive polymer composition so that covalent bonding with the topcoat may also occur.

Water repellant compounds for inclusion in the topcoat composition may include waxes, silicones, organofluoride compounds such as polytetrafluoroethylene. Preferred among such repellants is carnauba wax, beeswax, olefinic wax, paraffin. Polyurethanes, polyureas, polyesters, polysilicones and combinations thereof may also constitute constituents of the topcoat composition. Preferably, these polymers have significant numbers of non-reactive functional groups distributed throughout their polymer chains and as pendant groups so that hydrogen bonding, dipolar interaction and ionic interaction with the underlying films on the hair are produced. The presence of such polymers adds water repellency, shine and reasonable buoyancy character to the hair.

Hair setting compounds for inclusion in the topcoat composition may be readily removable with ordinary shampoo washing or may be long lasting in that multiple shampoo washings slowly will remove the hair setting compounds. The hair setting compounds enable retention of a particular set or coiffure under typical environmental conditions, such as rain, humidity and wind. Nevertheless, they may be removed by shampoo washing with commercially available shampoo formulations. The hair setting compounds useful for inclusion in the topcoat composition may be copolymers of an acidic vinyl monomer such as (meth) acrylic acid, a hydrophobic nonionic vinyl monomer such as alkyl (meth)acrylates, and first and second associative monomers such as polyoxyalkyenyl fumaric or similar unsaturated dicarboxylic acids. The compounds may be polyvinylpyrrolidones (PVP), copolymers of PVP and vinyl acetate (VA), acrylate and hydroxyalkyl acrylate copolymers, CARBOPOL (polyacrylic acid), CARBOPOL ETD polymer, xanthan gum, hydrophobically modified cellulose. Still other substances useful as hair setting compounds and as repellant compounds for topcoats are based upon (meth)acrylic copolymers of (meth)acrylic esters of C₆ to C₂₀ alkyl groups and (meth)acrylic esters of unsaturated alcohols and hydrophilic monomers such as (meth)acrylic acid. Copolymers of this formulation have unsaturation sites as films applied to the hair. A short UV irradiation of such copolymers as films enables cross linking and conveys wind, rain and shampoo resistance to the topcoat composition. Block copolymers of (meth)acrylic acid, crotonic acid, alkyl (meth)acrylates and minor percentages of olefinic monomers such as styrene provide the holding, low tackiness and high humidity resistance qualities to the topcoat while at the same time enabling readily removal with shampoo washing. Incorporation into the topcoat of a non-tacky pressure sensitive adhesive such as a copolymer of butyl acrylate and methacrylic acid with the percentage of methacrylic acid being minor on the order of 2 to 4 wt % also promotes hold and set. In some instances, a topcoat formulated with (meth)acrylate copolymers that are not readily removable by shampoo washing and display thermoplastic qualities at temperatures about at least 20° C. above human body temperature may be useful for reset of hair styles. This version of the topcoat may be warmed with a warm hair dryer and the hair reset to a new style. Cooling the reset hair provides the reset hair style as the thermoplastic polymer retains the shape provided by the reset.

The topcoat composition may contain the polymer compounds as microparticles dispersed in the medium or may be dissolved in solution with the medium. The topcoat optionally may contain pigment particles and dispersant as described above. The topcoat may be applied as a liquid composition using a brush, sponge or other similar applicator to coat individual hair strands. Alternatively, the topcoat composition may be incorporated into a spray pump container and applied as an aerosol to the hair. Applied as a spray the topcoat composition preferably is formulated to remain liquid on the hair for a sufficient time to enable gentle brushing to transfer the liquid throughout the hair strands and enable essentially all hair strands to be coated. When hair styling is part of the topcoat process, the hair may be set with mechanical devices or may be set with heat and mechanical manipulation as described above.

### Post Care Composition

The post care composition typically may be applied by the user periodically to preserve the shine, color lastingness and character of the color coating of the hair. The post care composition incorporates ingredients that impart lubrication, feel modifiers, sacrificial semi-fluid films to the hair. Includes are non-ionic surfactants, cationic surfactants such as long chain quaternary ammonium compounds, amosilicone conditioners, fatty acid amide conditioners, fatty alcohol betaines and sultaines, non-penetrating surfactants with a molecular volume larger than about 450 cc per mol. The post care composition may be formulated in a medium such as an aqueous or aqueous alcoholic medium that is capable of volatilization over a short period of time, such as one to five minutes. The post care composition may be applied to keratin fibers as a spray or as a liquid. Also useful as a post care composition is a protective composition that may be applied as a mask to the skin and parts of the user that are not to be treated with the compositions described herein. The protective composition forms a thin film mask on the skin and is readily removable by peeling. Adhesion to the skin is minimal so that peeling does not injure the skin. Compounds in aqueous alcohol solution provide the mask film upon evaporation of the medium. Compounds such as polymers and copolymers of high Mw organic hydroxy acids such as lactic and glycolic acid provide useful peelable masks. The post case composition can be designed to specifically care for the coating on the surface, versus the surface itself. So for example in the context of a coating on the hair surface, rather than use a regular product that is designed to clean and condition the hair surface, the post care composition is tailored to look after and care for the coating upon the hair surface.

### Solids Content

Embodiments of the color composition and pre-treatment composition include solids and liquids. The solids comprise any substance or material of these compositions that in a form uncombined with any other material, solvent, liquid or substance is has a solid physical form at ambient conditions. Included at least are the polymers and associated reaction materials and the pigment microparticles of these compositions. The medium, in contrast is a liquid and functions as a solvent and/or a liquid in which solid particles are dispersed. The optional components as well as the plasticizer, dispersing agent, surface treatment agent, cross linking agent and other materials added to the medium, if any, are included in the solids content as long as they remain with the polymers, reaction materials and pigment microparticles following application and setting of the color composition and pretreatment composition as a coating on strands of human hair. This includes substances that ordinarily would be regarded as liquids because they would remain in the coating on strands of hair.

The solids content of the color composition and pretreatment composition may range from about 0.1 wt % to about 50 wt % relative to the total weight of the respective composition. A preferred solids content ranges from about 0.1 wt % to about 20 wt % and another preferred solids content ranges from about 0.2 wt % to about 12 wt % relative to the total weight of the composition. An especially preferred solids content range is about 0.3 wt % to about 11 wt % with contents of about 1 wt %, about 2 wt %, about 3 wt %, about 4 wt %, about 5 wt %, about 6 wt %, about 7 wt % and about 8 wt % being more especially preferred.

### Testing the Flexibility of a Coating

With the color coating prepared on a releasable substrate and isolated as a standalone film can also be tested for optical density to check that the polymer film does not itself alter the hair appearance of the hair too significantly.

Further the polymer film preferably can tested to reveal its a glass transition point (Tg) as described above so that it is possible to prevent the colored coating from being damaged or cracked and to secure washing and friction remanence.

The color coating can have a surface energy between about 20 and about 50 mN m⁻¹. The color coating preferably has high transmission, to ensure that it does not interfere with the optics of the hair color. The polymer preferably has a refractive index between 1.4 and 1.6.

Ultimate elongation. The term ultimate elongation refers to the amount of elongation a given material can experience under a specific test method before failure occurs and the material breaks into more than one piece.

Young's modulus. Young's modulus, or the Young modulus, is a mechanical property that measures the stiffness of a solid material. It defines the relationship between stress (force per unit area) and strain (proportional deformation) in a material in the linear elasticity regime of a uniaxial deformation.

Ultimate compression. The term ultimate compression refers to the amount of compression a given material can experience under a specific test method before failure occurs and the material breaks.

Average repeated elongation before failure. Refers to the number of elongations to a fixed level of elongation repeat cycles that can be performed on a test material before failure occurs and the material breaks into more than one piece

The mechanical properties of the elastomer (Young's Modulus, Ultimate elongation, Ultimate compression, average repeated elongation before failure) are measured in a known manner using a Texture Analyzer TA.XT.Plus (Stable Micro Systems).

For the Young's Modulus and Ultimate elongation, the elastomer is prepared as a continuous film, for example 10 Mil thick, on a release layer (for example baking paper) using a BYK square applicator or bird type film applicator (for example 5570 Single Bar 6″, 10 mils or 5357 or Square Frame 4″, 5-50 mils). If the elastomer is produced a diluted system, those skilled in the art will select the appropriate thickness of the drawdown film to produce a suitable film for testing. The film is left to cure at 25° C. for −24 hours. The elastomer is removed from the release layer and cut into rectangular sections measuring 30 mm by 10 mm using a scalpel. The thickness is then measured using a calliper to account for any shrinkage or solvent loss during curing. The rectangular film is then attached to the TA instrument using (name of part used) within an initial separation of 12 mm. The sample is then elongated at a rate of 0.5 mm s−1 until the elastomer sample breaks. The Young's modulus is defined as the initial slope of the linear portion of the force-elongation curve, which occurs just after the initial force of 5 g is applied. As the initial cross-sectional area is known, the force is converted to MPa to calculate the Youngs modulus. The ultimate elongation is indicated as a percentage, i.e. extension distance at break/initial distance*100. To assess the average repeated elongation before failure, first the ultimate elongation of the sample is measured. The sample and TA instrument are arranged in the same way as to assess the ultimate elongation. The sample is then elongated at a rate of 0.5 mm s⁻¹ to a fixed elongation of 60% of the measured ultimate elongation. The sample then returns to its original state at a rate of 0.5 mm s−1 and the cycle is repeated until the sample breaks, or until a maximum of 2000 repeat cycles.

For the Ultimate compression, the elastomer is prepared as a continuous 3 mm film on a release layer. The film is left to cure at 25° C. for −24 hours. The elastomer is removed from the release layer and a series of cylindrical disks are punched out of the film with a 3.5 mm diameter. The thickness is then measured using a caliper to account for any shrinkage or solvent loss during curing. The rectangular film is then attached to the TA instrument using (name of part used) using a compressive cycle. The sample is compressed at a speed of 0.05 mm s−1 until the sample breaks. This is observed as a rapid deflection within the stress strain graph during the compressive cycle.

For all of the above mechanical property measurements results given are an average of at least 7 measurements.

The color compositions including the preformed polymer composition and the first and second components of the reactive polymer composition may be maintained in separate storage compartments or in separate kit form especially if the first and second components of the reactive polymer composition will react together without special activation. Additionally, the substantive constituent of a fourth component for reactive polymer activation if required may be maintained separately if it will catalyze or otherwise cause reaction of such functional groups. A convenient storage means can be utilized such as plastic squeeze tubes, plastic bottles, glass containers, sachets, multi-compartment containers, tooles, spottles syringes and plunger operated dispensing devices. Unit amounts for combination can be formulated so that the entire contents of a unit of the first component can be combined with the entire contents of the second component for application to the keratin fibers. Alternatively, metered or calibrated dispensing containers for providing measured amounts of the components as directed by printed instructions can be provided. With some embodiments, multiple components can be pre-combined for storage and handling as long as a substantive constituent that would cause in situ linking is maintained in a separate compartment.

Use of the foregoing delivery means enables preparation of an embodiment for practice of the method of the present invention. This embodiment may comprise sequential, simultaneous or premixed application, to keratin fibers, of the pretreatment composition and the color composition as the preformed polymer composition and/or the reactive polymer composition as first and second components. Pigment microparticles may be incorporated in the color composition. This aspect of application provides an underlayer of pretreatment composition and overlayer of preformed polymer composition and/or combined first and second components of the reactive-polymer composition on the keratin fibers. Management of the medium removal, temperature of the applied compositions and use of activation agents, if any, will enable transformation to a coating in which the polymers of these compositions in situ interact to covalently, hydrogen bond, electrostatically, coordinately, ionically, dipolar-wise, or entanglement-wise connect as the completed coating. Preferably, for the reactive polymer, the pairs of first and second functional groups are chemically reactive so that covalent and/or coordinate bonds are formed between the first and second components. More preferably, these pairs also combine with groups of the polymer of the pretreatment composition and the preformed polymer, the polymer product of the reactive polymer and the polymer of the pretreatment composition hydrogen bond and dipolar interact to bind all polymers together. With this aspect, the resulting color coating on keratin fibers provides good remanence against repeated shampooing, rinsing and contact with mild detergents, soap and similar wash substances.

### Application of Pretreatment Composition

According to the present invention, application of the pretreatment composition to keratin fibers as a pretreatment before application of the one or more color compositions is one of the prime factors for achievement of the qualities and characteristics of the color coating on keratin fibers. According to this embodiment of the method, the pretreatment composition with or without pigment, and preferably without pigment, is applied on or to at least a portion of the keratin fibers such as hair, and preferably throughout the keratin fibers. It is a key aspect of the invention that application of the pretreatment composition enables enhancement of adhesion between the hair, pigments and the color compositions. The amine polymer of the pretreatment composition interacts by hydrogen bonding and dipolar interaction with complementary keratin protein groups on the keratin fibers. The amine polymer also interacts by hydrogen bonding, dipolar interaction and molecular intertwining with corresponding hydrogen bonding, dipolar interaction groups of the color composition and by molecular intertwining to promote tight electrostatic and molecular entanglements. It is believed that these chemical interactions, which may be covalent, coordinate, electrostatic, ionic, dipolar and/or entanglement in nature function as melding between and among the keratin fibers, the pigment microparticles, and the polymers of the preformed polymer composition, the reactive polymer composition and the pretreatment composition.

Pretreatment with the pretreatment composition may be carried out prior to application of the color composition. Pretreatment may be carried out immediately prior to application of the color composition, or at least 1 hour prior to application of the color composition, or at least 24 hours prior to application of the color composition, or at least 10 days prior to application of the color composition, or at least one month prior to application of the color composition. Preferably, pretreatment may be carried out immediately prior to or within a few minutes up to an hour before application of the color composition. Typically, the pretreatment composition is at least partially dried with optional heating to at least substantially remove or otherwise eliminate the medium of the third component. For example, excess medium form the pretreatment composition on the hair may be removed by contacting the wet coated hair with an absorbent fabric or the wet coated hair may partially dried by heating with a hair drier. Preferably, substantial removal of the medium of the pretreatment composition is accomplished before application of the color composition.

In one embodiment more than one pretreatment composition may be applied to the hair. It may be that two different pretreatment compositions are applied sequentially, to provide a cumulative benefit for the subsequent composition which is then applied, or it may be that two different compositions are applied to substantially different portions of the hair. Such a case may arise when applying to hair which has quite different properties. For example to sections which have been pre-bleached or color, versus natural hair, or for root versus tip hair. In such cases different pre-treatments may be needed to prepare all of the hair for subsequent compositions. While such different pre-treatment would be directed to different portions of the hair, its likely that there would be a least some minor mixing and some areas of the hair would receive both pretreatment. A third case could be where a pre-treatment is applied to a section of the hair, for example at the roots, and the second pretreatment is then applied across all of the hair.

### Application of Color Composition Following Pretreatment

As described above, the one or more color compositions may be applied to the treatable material in combination with the foregoing pretreatment with the pretreatment composition. Embodiments of the color composition as the preformed composition may be maintained in combination with medium ready for dressing the pretreated keratin fibers, such as hair. Embodiments of the color composition as the the first and second components of the reactive-polymer composition are maintained separately when the first and second functional groups constitute reactive pairs as described above. Application of the one or more color compositions to pretreated keratin fibers may be preferably accomplished by sequential application to segments of the hair. Once all segments are coated with one or more wet color compositions, the one or more color compositions may be dried and/or cured to form overlaid coating layers on the keratin fibers. When the reactive-polymer composition is applied as the color composition, sequential application of the first and second components or simultaneous application of these components to the hair may be made. Typically for sequential application, either of the first and second components may be applied first, preferably the first component is applied first, especially for embodiments including pretreatment with the pretreatment composition. If segments of the hair are to be sequentially treated, a segment being treated receives both of the first and second components before another segment is treated. Alternatively, the first and second components may be mixed together to form a premix immediately before application to the treatable material. Typically, the rate of reaction of the first and second components and rate of drying may be pre-adjusted through medium control, pH adjustment if needed, concentration, steric interaction, temperature, and similar factors controlling reaction and/or drying rate so that a premix of the reactive composition preferably will not substantially interact before the premix is applied to the keratin fibers and the preformed composition preferably will not dry so fast that its binding interaction with the pretreat coating and/or keratin fibers is minimized or prevented. The practice of this step with the pre-treatment embodiment initially introduces combined first and second components or preformed composition on top of the pretreatment layer of amine polymer on the keratin fibers. Because the first and second components and the preformed composition are in a medium, penetration, combination, mixing and/or melding of the first and second components or preformed composition into the pretreatment layer will be accomplished at least in part. The penetration is believed to enable the linking among the reactive polymer or preformed polymer, the amine polymer and the keratin fibers. If a preformed polymer composition is also to be applied in combination with the reactive polymer composition, it typically may be applied following application of the reactive polymer composition. However, drying and curing of these compositions preferably occur after all compositions have been applied. In this manner, melding among all layers is best achieved.

Application of the one or more color compositions to keratin fibers pretreated with the pretreatment composition is preferably carried out after pretreatment. This sequence may be carried out immediately after pretreatment, or at least 1 hour after pretreatment, or at least 24 hours after pretreatment, or at least 10 days after pretreatment, or at least one month after pretreatment.

The sequential, simultaneous or premix application of the first and second components or the preformed composition may be applied to at least a portion of the keratin fibers or may be applied all over the keratin fibers. The portions of first and second components or the preformed composition may be applied sequentially, simultaneously or as a premix in a single application over all the keratin fibers or may be applied step-by-step to the keratin fibers. The first and second components or preformed polymer composition may be applied step-by-step, for example, in case the keratin fibers is damaged. Applying the first and second components or the preformed composition in a step-by-step manner as described above, may help to ensure that the treatable portions of the keratin fibers are saturated with the combined first and second components or preformed composition and may therefore provide a better coverage of the keratin fibers.

### Manipulative Techniques for Application

After each application of the pretreatment composition and one or more color compositions have been accomplished, and the wet coated keratin fibers, e.g. treated keratin fibers, optionally rinsed, the treated keratin fibers can be dried. The keratin fibers can be dried using an elevated temperature. The temperature of the keratin fibers can be increased to elevated temperatures above room temperature such as 40° C. or higher, for example using a hair drier. While the keratin fibers are being dried, some form of interdigitated implement can be used to help separate portions of the keratin fibers, and especially separate hair strands from one another. Examples of interdigitated devices include a comb or a brush. The keratin fibers can be dried with a hair drier while simultaneously being combed or brushed until it is dry to the touch. Alternatively, other means can be employed to dry and separate the keratin fibers such as hair simultaneously. For example, using a combination of air movement and vibrations will accomplish distribution of the multicomponent composition throughout the strands of hair.

### Operational Method for Coating Hair

The performance of operational method aspects of the present invention can be applied to keratin fibers to form a coating of the pretreatment and color compositions and optional topcoat composition. This aspect of the invention concerns a method for coloring keratin fibers and comprises applying embodiments of one or more pretreatment and color compositions for a time sufficient to deposit an effective colored coating on the keratin fibers such as each keratin fiber or hair strand. A somewhat to substantially overall distribution of the coating on the length and circumference of each fiber is produced.

To accomplish this aspect, embodiments of the pretreatment and color compositions are applied to the keratin fibers according to the sequences described above by brushing, painting, spraying, atomizing, squeezing, printing, rubbing massaging or in some manner coating the keratin fibers such as hair strands with the embodiments. Following application of a compositional embodiment to the keratin fibers such as hair strands, the composition is set, cured, linked, coordinated and/or otherwise melded together preferably by warming with blown warm air from a hair dryer or similarly treatable to remove the medium, initiate in situ linking of the reactive polymer and amine polymer as well as hydrogen bonding, molecular entwining and polar interactions among the preformed polymer, the product of the reactive polymer, the amine polymer and keratin fibers. The setting leaves a substantial to essentially complete overall bonding and binding among these substantive constituents of the color coating on keratin fibers.

The rate or rate of reaction for the reactive polymer composition to cure and to bond with the pretreatment composition is the speed at which reactants are converted into products. While not being a rate of covalent bonding, the preformed composition also is appropriately managed to form a film exhibiting adherence through hydrogen bonding, dipolar interaction, ionic bonding and entanglement. If the drying process occurs very fast, sufficient time is not allowed to enable the translational movement of the preformed polymer to provide non-covalent bonding. A very rapid drying may provide a readily peelable film of the preformed polymer. Therefore, in the context of the preformed and reactive polymer compositions forming adherent colored coatings, the rate refers to the speed at which the covalent and non-covalent bonding occur. In one embodiment it is preferred that the rate of reaction/drying is not so fast that the resulting elastomer forms before the wetting and spreading on keratinous surface can occur. If the rate of reaction/drying is too fast the resulting elastomer may not then be able to subsequently wet and spread on the hair surface, resulting in an inferior coating of the hair and one that displays less resistance to washing. In contrast, a rate of reaction/drying that is extremely slow will not enable a practical result in typical times for salon coloration treatment. In a preferred embodiment the rate of reaction/drying is slow enough such that the preformed composition/reactive polymer composition can wet and spread on the keratinous surface, yet also fast enough that when it has formed a macroscopically continuous film on a keratinous surface it bonds/binds covalently/non-covalently to produce an elastomer which is resistant to washing in a reasonable period of time, preferably in less than 48 hours, more preferably in less than 24 hours, even more preferable in less than 12 hours and most preferable in less than 6 hours under normal room temperature conditions.

The bonding and binding of the substantive constituents of pretreatment and one or more color compositions and the keratin fibers during application provides a color coating that resists removal by washing with dilute mixtures of soap and water or shampoo and water. Color remanence is developed so that washing with dilute aqueous soap solution or dilute aqueous shampoo will not substantially remove the coating, but the coating can be facilely removed by use of a transformation trigger. The properties of the coating include remanence, flexibility, adhesion, abrasion resistance and remanence which are due at least in part to the binding and bonding character of the substantive coating constituents including at least their intermolecular entwining, ionic and electrostatic intermolecular interaction, covalent and/or non-covalent linking, hydrogen bonding, dipole interaction and lipophilic interaction of these substantive constituents.

Selection of the substantive constituents of the various compositions can be made on the basis of properties such as a solid lattice formation and interaction with the pigment microparticles. Such properties include the flexibility, the hardness, the adhesion, the remanence, the resistance to water or to other chemical compounds, and the abrasion resistance. It is also possible to take advantage of the more versatile properties of block polymers (polymers comprising at least two distinct polymer segments), grafted polymers (polymers containing a polymeric side chain grafted onto the homopolymer or copolymer backbone), or random copolymers (polymers comprising at least two different monomers). In the block copolymers, for example, the amount of hard and soft blocks has a significant impact on the properties of the polymer.

The pretreatment and color compositions and optional topcoat in accordance with the present disclosure can have a viscosity that can be controlled to enable the product to be applied to the hair using either a brush and bowl or a bottle, but with sufficient rheology such that it does not drip and run from the hair onto the face or body.

Alternatively, low viscosity formulations may be applied to the hair via a suitable application device such that it does not drip and run form the hair onto the face and body.

The pretreatment and color compositions and optional topcoat can be utilized in concentrated form or in serial dilutions, to provide for a consistent color results substantially along the entire length of the keratin fibers.

The aspect of coloring mammalian or synthetic keratin fibers with a pretreatment and color composition and optional topcoat as described above includes a method for this coloring. The method comprises:
(i) applying the above-described pretreatment and color compositions to keratin fibers to obtain an effective, deposited coloring amount of the combined composition including pigment microparticles and optional additional components;
(ii) setting the pretreatment and color compositions by removing or otherwise eliminating the medium (e.g., by drying the composition); and.
(iii) setting the interaction among the first and second components of the pretreatment and color composition by initiating the in situ linking among these groups.

During the setting/drying step, color distribution can be facilitated by concurrently moving and/or stroking the hair with an interdigitating device. Interdigitating devices include a comb or brush. The interdigitating device needs to be pulled substantially along the hair strands from root to tip. It can be pulled through at a rate of 0.1 cm s⁻¹ to 50 cm s⁻¹ or at a rate between 0.5 cm s⁻¹ to 20 cm s⁻¹

The pretreatment and color compositions and optional topcoat are applied to the mammalian or synthetic keratin fibers in any suitable way including spraying the pretreatment and color composition, massaging the keratin fibers by hand, after applying the pretreatment and color composition to the hand or by combing, brushing or otherwise applying the pretreatment and color composition throughout the mammalian or synthetic keratin fibers.

The methods by which the pretreatment and color compositions and topcoat composition described herein are applied can be modified, such that the user applies the product in one region of the hair, and then can apply a diluted version in another region of the hair. The dilution formula is specially chosen to be compatible with the colorant formulation and reduces the coloring strength, while maintaining the longevity of the color result. This can effectively be a “blank” formulation, which contains broadly the same materials as the coloring formulation, but with lower or no pigments present. When diluted the ratio of the diluent to colorant can be between about 10:1 and about 1:10, about 8:1 and about 1:2 or about 5:1 and about 1:1.

Alternatively, the amounts of pretreatment and color compositions and topcoat composition applied can be altered in different regions of the hair, for example half the product is applied in the lengths of the hair, leading to a less colorful result. The difference in amounts applied in one region of the hair versus another can be between about 4:1 and about 1:4 or about 2:1 and about 1:2.

Alternatively, a combination of this approaches may be used to deliver the target color variation.

When the foregoing techniques are not possible to be applied, rather than apply a single hair color, it may be possible to apply two or more hair colors to different regions of the hair. When this is done, the different hair colors preferably provide complimentary colors so as to develop an attractive result. The difference in colors that can be used, based on the end result on hair tresses (as described later—natural white hair non-pre-bleached) are as follows. As described within the CIELCh system:

$$\begin{matrix}Color 1 ( LCh ) versus Color 2 ( LCh ) \\ Color 1 L - 15 < Color 2 L < Color 1 L + 15 \\ 0 or Color 1 C - 10 < Color 2 C < Color 1 C + 10 \\ Color 1 h - 45 < Color 2 h < Color 1 h + 45\end{matrix}$$

Those skilled in the art of color measurements will know how to interpret difference in hue angles, h, when they extend from low positive values to those near to 360 degrees due to the periodic circular nature of the hue angle.

The method for use of the pretreatment and color compositions and optional topcoat composition in accordance with the present invention can occur during any suitable period. The period of application can be from about 0 to 30 minutes, but in any event a period that is sufficiently long to permit the coating of pigment microparticles to coat and adhere or bind to each separate keratin fiber, substantially along the entire length of each keratin fiber. The resultant is keratin fibers having a color and permanence that is at least equivalent to the color resulting from oxidative hair color, except under much milder conditions.

The pretreatment and color compositions described herein can be prepared by the manufacturer as a full shade, e.g., one that is ready to apply to the hair, and then shipped as a discrete unit to the user. The user may need to re-blend the pretreatment and color composition prior to application to ensure that the pretreatment and color composition delivers the optimum performance. Such re-blending can require shaking the pretreatment and color composition for about 1 to about 120 seconds or from about 3 to about 60 seconds. Reblending may also be performed by stirring the pretreatment and color composition prior to use. This may occur for about 1 to about 120 seconds or from about 3 to about 60 seconds. Although the pretreatment and color compositions according to the present invention are designed to provide stable suspensions of the pigment particles, the re-blending to agitate the microparticles and resuspend them in a substantially uniform distribution is desirable.

Multiple compositions comprising different pigments can be blended together prior to application to the keratin fibers. Such blending can be done in a manner so as to apply a plurality of complementary surface colors to the keratin fibers.

The pretreatment and color compositions can include multiple layers, involving multiple applications of at least the first and second components following the first application of the three components. It may be beneficial also to periodically reapply the third component. The techniques for applying multiple layers follow the techniques described above for application of a single pretreatment and color composition.

The coating of pigment microparticles comprising at least one pigment in a coating of the substantive constituents of the pretreatment and color compositions can be adhered to the treatable material such as hair utilizing a coating having a total thickness at any given point along the hair fiber of less than about 5 m, preferably less than about 2 m as measured using a scanning electron microscope (SEM). To make such measurements, a coated hair sample can be embedded in a suitable resin, and then sectioned root to tip using techniques known to those skilled in the art of scanning electron microscopy. The thickness of the layer on the surface can then be assessed along the line of cuticles over a length of at least 100 m. The thickness of layer is determined by averaging 10 points evenly spaced over the section of interest.

In the course of application of the pretreatment, color compositions and topcoat compositions, it is possible to dress portions of hair rather than as one whole uniform area from root to tip and across the regions of the scalp. These portions may be up to the size of the one whole entire area but no two portions of a person's hair are the same. For this reason, if one portion covers the one whole uniform area, a second portion cannot also cover the one whole uniform area. The two portions do not necessarily need to cover the one whole uniform area. Several nonlimiting examples help explain a few of the many possible first and second portions of the hair. In a first example the first portion of the hair refers to the hair adjacent to the persons scalp, the so called root hair region which may extend from a few millimeters to several centimeters. In this example the second portion of the hair is the hair which is not adjacent to the scalp, i.e. the area which is not within the first portion. There may be some overlap between the first and second portions, due to the limitations of physically segregating these two portions on a person's head, but the two portions are different to one another. In a second example, the first portion of the hair refers to the one whole uniform area from root to tip. In this example, the second portion of the hair is then a smaller regions of the hair, for example the area adjacent to the scalp. Again. these two portions are different to one another, but have a significant area of overlap, with the second portion covering an area which completely overlapping with the first portion, but where the first portion has a large area which does not overlap with the second portion.

This understanding of the different qualities and attributes of sections of hair on a person's scalp shows that it is appropriate and preferably to apply separately pretreatment and color compositions to sections of hair strands. In addition to varying the concentration of the pigment microparticles and optional coloring agent, different shades and/or colors of pretreatment and color compositions can be applied to different sections of a strand of hair or a group of strands of hair. For example, the hair roots, mid sections and tips sometimes or often have different shades of color in their natural condition. This variation can be mimicked, altered or covered through use of differing shades or colors of the pretreatment and color compositions. Roots, for example can be covered with a lighter shade and the tips can be covered with a darker shade to produce a two tone variation of the hair. Application to the hair of a first portion of pretreatment and color composition followed by stripping the composition from the hair mid sections and ends followed by setting the remaining composition on the hair roots will provide a first hair color coating on the roots. The mid-sections and tips can be dipped or brush applied with a second portion of pretreatment and color composition to complete the two color or two tone treatment. The use of multiple pretreatment and color compositions to produce multiple coatings on the hair can provide overlapping, sequential or coterminous coatings on the hair according to typical and routine techniques for applying multiple versions of hair color practiced by professional hair salons.

### Post Treatment

An optional post treatment composition can be applied after treating the treatable material such as hair with the multicomponent compositions described herein. This can be applied either directly after completion of coloring with the multicomponent composition. The post treatment can be either single application or multiple application across time. The post treatment can be used to improve one or more of: feel, resistance to shampoo/conditioner/water washing treatments, and shine of the hair. Nonlimiting examples of materials used to improve the feel are those which impart lubricity to the treatable material such as hair strands and/or help the hair strands separate during the drying steps. These materials include, for example silicone conditioners, silicone polyethers, silicone polyglucose, polyisobutene, copolymers of ethylene and propylene oxide, and commonly used cosmetic oils and waxes. Nonlimiting examples of materials used to improve shampoo wash resistance are materials which act as a ‘sacrificial layer’ for example polymeric silicones and their copolymers, silicone resins, cosmetics oils and waxes. Nonlimiting examples of materials used to improve the shine of hair (meaning a decrease of the full width at half maximum parameter of the specular reflection curve as measured by a goniophotometer) are those materials which form a smooth film above the previously applied pigment polymer composite on the hair. In general, any cosmetically known film forming material can be used, but preferred are materials such as polymeric silicones and polycationic materials.

### Removal of Color Coating

Hair colorants made from the pretreatment and color composition are very resistant to everyday hair treatments (such as washing with shampoo, conditioner etc) can be removed via use of specifically designed “removal formulations.” These are specific chemical mixtures, described herein, and are designed to work by one or both of two broad mechanisms: cleavage of chemical bonds, either linking groups in one or both of the combined first and second components or bonds within the first component, second component of preformed polymer and solvation of components of the colored coating.

First, the mixture can be made to be a solvent for the pigment itself. In this case, the mechanism of removal involves first dissolution of the pigment from the binding matrix, followed by removal from the hair by rinsing with water or some other carrier. In this case it is believed, whilst not being bound by theory, that the chemical nature of the pigment, even when in dissolved form, is such that there is minimal attraction/solubility in the hair matrix itself, thus allowing removal of the color.

Second, the “removal formulation” can be made such that it dissolves, weakens or chemically breaks down the polymer coating holding the pigment on the hair. In this case it is believed, whilst not being bound by theory, that the pigments embedded in the binder matrix are released due to weakening or dissolution of the coating itself and, because the coloring material is a pigment, it has minimal attraction for the hair surface and is too big to penetrate the hair, and in consequence this facilitates removal of the color.

The combination of the above mechanisms will also provide the desired result of removal of the color.

Attacking the functional group bonds of the polymer network of the coating on the treatable material such as hair can have a dramatic impact on the properties of the coating which is adhered to the surface. An agent that cleaves those bonds can act as a trigger agent to divide the polymeric network and enable surfactant and solvent to readily disperse the cleaved coating. Such agents include basic amino alcohols such as dimethylaminoethanol (dimethylethanolamine, DMEA), dimethylaminopropanol, and similar amino alkanol agents such as monoethanolamine, diethanolamine and triethanolamine. These amino alcohols can be formulated in aqueous medium to enable coating removal. Additionally, or alternatively, fatty organic acids such as dodecylbenzene sulfonic acid or oleic acid may be combined with non-aqueous medium such as a volatile, harmless hydrocarbon including but not limited to dodecane to trigger removal. These organic acids function as surfactants to lift the coating from the treatable material surfaces and to break the functional group bonds which cleaves the polymeric network of the coating. The concentration of the trigger agent in alcoholic medium such as methanol, ethanol or aqueous medium or in non-aqueous medium may range from about 0.1% to about 15% by weight, preferably about 0.5% to about 10% by weight, more preferably about 1% to about 7.5% by weight relative to the total weight of the removal solution.

The organic or silicone polymer coating may also contain a group L, the linker group and L will typically contains a cleavable linkage, such as an ester, imide, amide and the like. This linkage is susceptible to hydrolysis and can be cleaved using basic or acid lysis. The cleavage will include a counter-nucleophile which can be water or a small molecular weight monofunctional amine or thiol.

The organic or silicone polymer having chain extensions with siloxane condensation, a strong acid such as dodecyl benzene sulfonic acid (DBSA) or a source of fluoride anion tetrabutylammonium fluoride (TBAF) in appropriate solvent as described in combination with Hansen solubility parameters including δd+δp+δh wherein δd is from 13 to 25, preferably 15-19 and δp is from 0 to 15, preferably 0 to 5 and 6 h is from 0 to 25, preferably 0 to 8.

Additionally, the in chain functional groups such as N-acylurea, urea, urethane, amide and/or ester can be cleaved through use of a small molecular weight monofunctional amine or thiol to attack the functional group and disrupt polymer chains.

Also, if silicone polymeric bridges are present in the silicone polymer or in the organic polymer, an organic acid (such as DBSA) may be used to de-polymerize the chain. TBAF or other organic fluoride such as Olaflur can also be used to de-polymerize the chair. It is also advantageous in all “off” techniques to employ an off reagent also has some surfactant quality.

When the pretreatment and color composition is applied to the hair, the multi-application process physically distributes the components to cover all of the hair. The spraying, massaging, combing and/or hand manipulating the pretreatment and color compositions produces the full coverage and at the same time leaves thin spots in the otherwise substantially uniform coating. This activity also will aid in the removal process.

Additionally, waxy non-reactive, non-combinable substances having melting points no greater than 20° C. higher than human body temperature may be incorporated into the pretreatment composition. The concentration of waxy substance may be sufficient to enable heat disruption of the polymer film of the pretreatment layer on the keratin fibers but not enough to prevent the engagement of the polymer film properties of the pretreatment layer. By warming the hair with a hair dryer at a temperature of more than 20° C. higher than body temperature, the waxy substance may be melted at least in part so as to disrupt the color coating on the keratin fibers. Combing or brushing can remove disrupted color coating.

Alternatively, an organic solvent soluble polymer such as a cellulose derivative, including but not limited to nitrocellulose, cellulose acetate-butyrate or other solvent soluble polymer may be incorporated into the color composition. The amounts and concentrations of the solvent soluble polymer are sufficient to enable the polymer to form separate domains of polymer film within the coating produced from color compositions. Contacting such a coating with an organic solvent in aqueous medium will at least in part dissolve the solvent soluble polymer and disrupt the continuous nature of the coating layer. Combing or brushing can remove disrupted color coating.

### Remanence and Treatable Material Inspection

Damage caused to the hair by application of the pretreatment and color composition and removal of the resulting coating can be assessed by FT-IR (Fourier Transform Infrared) method, which has been established to be suitable for studying the effects of keratin surface damage. Strassburger, J., J. Soc. Cosmet Chem., 36, 61-74 (1985); Joy, M. & Lewis, D. M., Int. J. Cosmet. Sci., 13, 249-261 (1991); Signori, V. and Lewis, D. M., Int. J. Cosmet. Sci., 19, 1-13 (1997)). In particular, these authors have shown that the method is suitable for quantifying the amount of cysteic acid. In general, the oxidation of cystine is thought to be a suitable marker by which to monitor the overall oxidation of the keratinous part of the fiber. Net, the measurement of cysteic acid units by FT-IR is commonly used.

Signori and Lewis (D. M., Int. J. Cosmet. Sci., 19, 1-13 (1997)) have shown that FT-IR using a diamond Attenuated Total Internal Reflection (ATR) cell is a sensitive and reproducible way of measuring the cysteic acid content of single fibers and bundles. Hence, the method that can be employed to measure the cysteic acid content of multiple fiber bundles and full hair switches, is based upon the FTIR diamond cell ATR method employed by Signori and Lewis (1997). The detailed description of the method for testing the different damage inhibitors follows thereafter:

A Perkin Elmer Spectrum® 1 Fourier Transform Infrared (FTIR) system equipped with a diamond Attenuated Total Internal Reflection (ATR) cell may be used to measure the cysteic acid concentration in mammalian or synthetic hair. In this method, hair switches of various sizes and colors can be used. The switches may be platted (−1 plait per cm) in order to minimize variations in surface area of contact between readings. The Oxidative hair Treatment Protocol described above may be repeated for 5 cycles to mimic the behavior of hair after repeated bleaching cycles. Following this treatment, four readings per switch may be taken (⅓ and ⅔s down the switch on both sides), and an average calculated. Backgrounds may be collected every 4 readings, and an ATR cell pressure of 1 N/m may be employed. The cell may be cleaned with ethanol between each reading, and a contamination check may be performed using the monitor ratio mode of the instrument. As prescribed by Signori &amp; Lewis in 1997, a normalized double derivative analysis routine may be used. The original spectra may be initially converted to absorbance, before being normalized to the 1450 cm⁻1 band (the characteristic and invariant protein CH₂ stretch). This normalized absorbance may be then twice derivatised using a 13 point averaging. The value of the 1450 cm⁻¹ normalized 2nd derivative of the absorbance at 1040 cm⁻¹ may be taken as the relative concentration of cysteic acid. This figure may be multiplied by −1×10⁻⁴ to recast it into suitable units.

When the compositions of the current invention can be applied to the hair and then removed there can be a non-significant change to the level of damage to the hair, whereas with conventional oxidative colorants there can be a large increase in the measured damage.

The instant disclosure is not limited in scope by the specific compositions and methods described herein, since these embodiments are intended as illustration of several aspects of the disclosure. Any equivalents are intended to be within the scope of this disclosure. Indeed, various modifications in addition to those shown and described herein can be within the grasp of those with ordinary skill in the art. Such modifications are also intended to fall within the scope of the appended claims.

### Color Selection

Also contemplated herein are pretreatment and color compositions having a given color area (gamut principle described above) defined by color coordinates (a*, b*) in the color space represented by the L*a*b* color system, which can be divided into a plurality of color areas. Each of the plurality of colors obtained from the area surrounding a given set of hair fibers is judged to belong to which color area of the colored area of a certain color. The number of colors judged for each color area is counted, and the color of the color area with the largest number of colors is selected as a representative color of the area surrounding a given set of hair fibers. The compositions are capable of delivering colors on hair (test method herein for fade) such that the results colors lie within the range of about 18<L<about 81, about −2<a<about 45, and about −13<b<about 70.

Also contemplated herein are pretreatment and color compositions that do not change the hair color, but instead change some other feature of the hair including shine (e.g., making it shinier or matte), the thickness of the hair and/or the feel of the hair.

When the color is removed from the keratin fibers the waste water/composition can be treatable to remove the pigments from the waste water effluent system. This can be achieved by filtration, or through cyclone technology, where the density differences are used to force the pigments to the settle, and the water to pass through.

Application of Composition to Hair Tresses Hair preparation: Two types of hair were used: un-damaged and damaged.
Un-damaged hair: Natural white undamaged human hair was purchased from Kerling International Haarfabrik GmbH, Backnang, Germany company in the form of 10 cm long and 1 cm wide strands. This hair was used as received. Natural dark brown, Level 4 hair was purchased from Kerling International Haarfabrik GmbH, Backnang, Germany company in the form of 10 cm long, 1 cm wide strands. This hair was used as received.
Damaged hair which was produced following this procedure: Natural white undamaged human hair was purchased from Kerling International Haarfabrik GmbH, Backnang, Germany company in the form of 10 cm long and 1 cm wide strands and was bleached. The strand was treated with a mixture of Blondor Multi-Blonde bleach powder available from Wella Professionals mixed 1 part with 1.5 parts of 12% Welloxon Perfect available from Wella Professionals. About 4 g of this mixture was applied to each gram of hair. The tresses were then incubated in an oven at 45 C for 30 minutes after which they are rinsed in water, 37+−2 C with a flow rate of 4 L/min for 2 minutes and the hair is then dried with a standard Hair dryer from Wella.
   Organic pigments were tested on the natural white hair as received and treated according to the protocol described above to assess the initial color and color remanence. TiO₂ and Metal flakes were tested on the dark brown hair described above to initial color and color remanence.
   Hair pre-treatment: Hair prepared as described above was treated with the pre-treatment composition described above, one gram of composition per one gram of hair. The composition was left on the hair between 1 and 5 min. The hair was then dried using a blow dryer to result in dry hair. Alternatively, the hair could be left wet, the excess of the composition was removed with an absorbent material, for example a towel.
   General Coloring Procedure: To the pre-treated hair tress described above is added a freshly prepared multicomponent coloring composition as described above, 1 gram per 1 gram of hair. Application is accomplished by a slow distribution and spreading on the hair tress, for example, with fingers, brush, comb or other manipulation instrument. The slow distribution can be accomplished by application with a syringe or a pipette serially to portions of the hair tress. Excess is removed with absorbent tissue material and the resulting colored hair tress is blow dried with combing using a hair dryer to achieve better hair individualization. The hair was then pulled 3 times through a flat iron at 150° C., 2 seconds for each pull of the tress through the flat iron. Treated hair tresses may be kept at rest for a time period as much as a day at room temperature or at least above 17° C.
   Standard wash procedure: The standard wash procedure is used to determine the remanence of the colored hair tresses.
Rinse the hair tress for approximately 10 seconds with water (4 L min¹) at approximately 37+/−2 C.
Apply 0.1 g “Wella Professional Brilliance Shampoo for fine and normal hair” without dilution to the individual colored hair tress weighing about 1 g described above.
Shampoo is worked into the colored hair tress in the absence of water dilution for 30 sec with fingers by using a stroking motion into the hair.
The shampooed colored hair tress is rinsed with water for approximately 30 seconds.
The rinsed colored hair tress is then dried using a hot blow dryer while mechanically separating the fibers in the substrate material until uniformly dry.
Steps 1-5 described above represent one cycle of the standard wash procedure.
Repeat of standard wash cycle for multiple cycles and comparison of the multiply washed hair tress to an unwashed colored hair tress which indicates the degree of color remanence using the Color Remanence Scoring Values described below.
   Remanence was assessed visually by comparing the washed samples versus a retained tress which had been colored but not washed. They were graded on a 5 point scale according to the following criteria. 1 no color left, 2 faint color, 3 washed-out color, 4 intense color with some color loss, 5 color unchanged versus reference.

### Color Removal Composition

General Procedure: Combine active agent with a medium, the medium may contain solvent, thickener, polyelectrolyte, detergent builders and combine and mix until uniform.

### Color Removal Application

Apply 1 gram of color removal composition to 1 gram of colored hair tress.
The color removal composition is worked into the colored hair tress in the absence of water dilution for approximately between 30 sec to 1 minute with fingers or a comb of manipulation instrument by using stroking motion into the hair.
Leave the color removal composition onto the colored hair tress for approximately 1 min or longer undisturbed.
Remove excess color removal composition with an absorbent tissue material.
Apply 0.1 g of “Wella Professional Brilliance Shampoo for fine and normal hair” without water dilution and work into the colored hair tress for 30 sec with fingers by using stroking motion into the hair.
The shampooed colored hair tress is rinsed with water for approximately 30 seconds.
The rinsed colored hair tress is then dried using a hot blow dryer until uniformly dry.
   To quantify color on hair tresses a Minolta 2600d using a SAV aperture was used. 5 measures were taken on each side of the tress and the color calculated using D65 lighting. Color calculation for dE76 and de2000 were performed using the equations well know to those skilled in the air.

### Data Processing and Computer Assisted Color Selection

The embodiments described herein may be implemented by computer software executable by a data processor of the apparatus, such as in the processor entity, or by hardware, or by a combination of software and hardware. Further in this regard, it should be noted that any blocks of the logic flow as in the Figures may represent program steps, or interconnected logic circuits, blocks and functions, or a combination of program steps and logic circuits, blocks and functions. The software may be stored on such physical media as memory chips, or memory blocks implemented within the processor, magnetic media such as hard disk or floppy disks, and optical media such as for example DVD and the data variants thereof, or CD. The data processing apparatus comprises:
a memory; and
at least one processor adapted to:
   obtain at least one parameters of the hair prior to coloring and at least one parameter of the multicomponent in situ linkable composition for coloring hair; determine at least one product from a database of products stored locally or remotely that will maintain the color for a long time;
   store the recommended product(s) in the memory;
   data stored either directly or remotely

Data may be stored directly within the memory of the data processor apparatus such as memory chips, or memory blocks implemented within the processor, magnetic media such as hard disk or floppy disks, and optical media such as for example DVD and the data variants thereof, or CD, or solid state drives. If the data is stored remotely it refers to data which in not stored within the data processor apparatus, but for example within the cloud or another server which the data processor apparatus can retrieve the data from.

### STATEMENTS OF EMBODIMENTS OF THE INVENTION

The following statements disclose embodiments of the invention directed to characteristics and properties of the compositions to be dressed to keratin fibers, additives for the compositions, methods for applying the compositions, kits for containing the compositions and the qualities, characteristics and attributes of the color coatings on keratin fibers. In the context of the invention, keratin fibers includes not only human scalp hair but also eyelash and eye brow hair. Especially preferred are compositions, methods and color coatings on keratin fibers as human scalp hair. The sections of the statements include compositions, polymer properties, dispersant, additives, pretreatment compositions, topcoat compositions, removal compositions, post care compositions, methods for application of color compositions cleaning methods, methods for application of pretreatment compositions, methods for application of care compositions, methods for application and use of removal compositions, color selection, color coating (film) properties, kits and systems.

The statements are organized into sections which correspond to and further illustrate and exemplify details of the forgoing compositions, methods and features of the invention described above. These sections also add further details and embodiments to each of above-described aspects of the invention. Each section is to be considered an addendum to the corresponding detailed section above. Some of the following sections such as kit and cleaning do not have formal detail sections in the foregoing discussion yet the concepts embodied are disclosed in the foregoing detailed disclosure. The invention includes these sections as additional aspects of the invention.

The statement sections include:
a) Compositions, Statements 1-11;
b) Polymer Composition Properties, Statements 12-23;
c) Dispersant, Statements 24-29;
d) Additives Compositions, Statements 30-40;
e) Pretreatment Compositions, Statements 41-60;
f) Topcoat Compositions, Statements 61-65E;
g) PostCare Compositions, Statements 1-11;
h) Protective Compositions, Statements 1, 2;
i) Removal Compositions, Statements 1-9;
j) Method for Applying Compositions, Statements 1A-5
k) Method for Cleaning, Statements 1-5;
l) Method for Applying Pretreatment Compositions; Statements 1-10;
m) Method for Applying Topcoat, Statements 1-5;
n) Method for Applying PostCare Compositions, Statements 1-2;
o) Method for Removal, Statements 1-12;
p) Preferences for Keratinous Surfaces, Statements 1, 2;
q) Method for Coloration, Statements 1-18;
r) Method for Applying Protective Layer, Statements 1-3A;
s) Color Selection, Statements 1-15;
t) Color Coating Properties, Statements 1-39
u) Kits, Statements 1-17;
v) Systems including Computer Aid, Statements 1-10.
   As discussed above, these sections constitute further disclosure details of aspects of the invention.

### Compositions

Statement 1, compositions. A composition for treating a keratinous surface, comprising: a preformed composition or a reactive composition wherein:
a) The preformed composition comprises a medium and a preformed polymer of organic, silicone or organosilicone configuration having multiple numbers of optional non-reactive functional groups distributed within the polymer chain(s) and/or distributed as pendant groups along the polymer chain;
b) The reactive composition comprises a medium and first and second components, the first component comprising an organic, silicone or organosilicone polymer having first reactive functional groups and the second component comprising an organic, silicone or organosilicone in situ linking material having reactive second functional groups wherein the first and second reactive functional groups are complementary reactive pairs and can form covalent linkages in situ to form an in situ polymer product of organic, silicone or organosilicone configuration; and the first and/or second component optionally have non-reactive functional groups that do not react with the complementary reactive pairs.
   Statement 2A, compositions. The composition of statement 1 or of any of the succeeding statements of pretreatment wherein the preformed composition and/or reactive composition and/or pretreatment composition further comprises pigment particles dispersed in the medium.
   Statement 2B, compositions. The composition of statement 2A wherein the pigment comprises organic and/or metallic microparticles providing a visual spectrum of colors and hues.
   Statement 2C, compositions. The composition of statement 2A or 2B wherein the pigment particles include at least one or more of Pigment Yellow 83, Pigment Green 36, Pigment Blue 60, Pigment Blue 66, Pigment Blue 16, Pigment Black 6, Pigment White 6, Pigment Red 122, Pigment Red 5, Pigment Red 112, Pigment Violet 19, Aluminum flakes.
   Statement 2D, compositions. The composition of any of statements 1, 2A, 2B or 2C or any of the succeeding statements of pretreatment wherein the preformed composition and/or reactive composition and/or pretreatment composition and/or any combination thereof further comprises one or more macro-dyes having a wt avg Mw of at least 1 KDa.
   Statement 2E, compositions. The composition of statement 1 and/or any of the succeeding statements of pretreatment wherein the preformed composition and/or reactive composition and/or the pretreatment composition further comprise any combination of organic pigment microparticles, inorganic pigment microparticles, metal microflakes and/or macro-dyes having a wt avg Mw of at least 1 KDa.
   Statement 3A, compositions. The composition of any of the preceding statements where the complementary reactive pairs are isocyanate/thioisocyanate and hydroxyl, amine or mercapto or any combination of hydroxyl, amine and mercapto; cyclic carbonate-silyl and amine; carboxyl and hydroxyl, amine or mercapto or any combination of hydroxyl, amine and mercapto; epoxy and hydroxyl, amine or mercapto or any combination of hydroxyl, amine and mercapto; cyclohexylepoxy and hydroxyl, amine or mercapto or any combination of hydroxyl, amine and mercapto; (meth)acryloxy and hydroxyl, amine or mercapto or any combination of hydroxyl, amine and mercapto; melonic anhydride and hydroxyl, amine or mercapto or any combination of hydroxyl, amine and mercapto; formyl and amine or mercapto or any combination of amine and mercapto; azido and alkynyl; vinyl and mercapto; mercapto and mercapto; carbodiimido and carboxyl or amine or a combination thereof, and any combination of —Si(R¹)ₐ(R²)₃₋ₐ wherein R¹ is alkoxy of 1 to 6 carbons or OH, an —OAc group, or a —O—N═CHR¹ group, R² is alkyl of 1 to 3 carbons and a is an integer 1, 2 or 3; or SiHR² and —SiR²—CH═CH₂.
   Statement 3B, compositions. The composition of any of the preceding statements wherein the complementary reactive pairs are isocyanate and hydroxyl, carbonate-silyl and amine; (meth)acryloxy and hydroxyl, amine or mercapto or any combination of hydroxyl, amine and mercapto; carbodiimide and carboxyl or amine; or alkoxy silyl.
   Statement 3C, compositions. The composition of any of the preceding statements wherein the first and second reactive functional groups comprise a carbodiimide complementary pair.
   Statement 3D, compositions. The composition of any of the preceding statements wherein the first and second components comprise organosilicone polymers and the first and second reactive functional groups are an alkoxy/hydroxysilyl condensation complementary pair.
   Statement 3E, compositions. The composition of any of the preceding statements wherein the first and second reactive functional groups are a polyisocyanate complementary pair or a cyclic carbonate complementary pair for urethane and/or urea functional group formation.
   Statement 3F, compositions, the composition of any of the preceding statements wherein the first and second reactive functional groups are (meth)acryloxy and amine or mercapto or any combination of amine and mercapto.
   Statement 4A, compositions. The composition of any of the preceding statements wherein the preformed polymer and/or the first and/or second components comprise oligomeric and/or polymeric silicone molecules.
   Statement 4B, compositions. The composition of any of the preceding statements wherein the preformed polymer and/or the first and/or second components comprise organic oligomeric and/or polymeric molecules.
   Statement 4C, compositions. The composition of any of the preceding statements wherein the preformed polymer and/or the first and/or second components comprise organosilicone oligomeric and/or polymeric molecules.
   Statement 5, compositions. The composition of any of the preceding statements wherein the in-situ linking material is a small molecule, oligomer or polymer having a carbon and/or silicone molecular construction.
   Statement 6, compositions. The composition of any of the preceding statements wherein the preformed polymer and/or the first and/or second components comprise non-reactive functional groups.
   Statement 7, compositions. The composition of statement 6 wherein the non-reactive functional groups comprise hydrogen bonding groups, ionic groups, electrostatic interaction groups and any combination thereof
   Statement 8, compositions. The composition of statement 7 wherein the non-reactive functional groups are unreactive toward the first and/or second functional groups of the first and/or second components.
   Statement 9, compositions. The composition of any of statements 6-8 wherein the non-reactive functional groups are urethane, urea, ester, amide, carbonate or any combination thereof.
   Statement 10, compositions. The composition of any of the preceding statements wherein the preformed polymer and/or the first and/or second component or both of the preformed polymer and the first and/or second component have in-chain trigger groups for cleavage of the chain.
   Statement 11, compositions. The composition of any of the preceding statements where the medium comprises a volatilizable aqueous medium, volatilizable aqueous-silicone medium, volatilizable aqueous-organosilicone medium, volatilizable aqueous organic or volatilizable organic medium and the organic medium comprises a volatile aliphatic alcohol, ester, ketone or hydrocarbon.

### Polymer Composition Properties

Statement 12, composition properties. The composition of any of the preceding statements of compositions comprising the preformed composition.
Statement 13, composition properties. The composition of any of the preceding statements of compositions comprising the reactive composition.
Statement 14, composition properties. The composition of statement 13 of properties, comprising the reactive composition wherein the rate of covalent linking between first and second functional groups is selected to enable wetting and spreading of said first and second components on the keratinous surface prior to curing.
Statement 15, composition properties. The composition of any of the preceding statements of properties comprising the reactive composition wherein the cure time of the first and second components is between 10 seconds and 24 hours, more preferably between 2 minutes and 18 hours, even more preferably between 10 minutes and 12 hours.
Statement 16, composition properties. The composition of any of the preceding statements of properties comprising the reactive composition wherein the polymer in the first component and/or the in situ linking material in the second component and/or combinations thereof have an average of three or more first and/or second reactive functional groups per species, more preferably an average of 4 or more first and/or second reactive functional groups per species, more preferably an average of 6 or more first and/or second reactive functional groups per species.
Statement 17, composition properties. The composition of any of the preceding statements of properties or any of the succeeding statements of pretreatment comprising the preformed composition and/or the reactive composition and/or pretreatment composition wherein the preformed polymer and/or the polymer in the first component and/or the in situ linking material in the second component and/or a pretreatment base compound and/or any combinations thereof have an average molecular weight of greater than or equal to their respective entanglement molecular weight.
Statement 18, composition properties. The composition of any of the preceding statements of properties and/or of any of the succeeding statements of pretreatment, comprising the reactive composition and/or the pretreatment composition wherein the one or more polymers in the first component and/or the in situ linking material in the second component and/or the pretreatment base compound and/or any combinations thereof have an average functional group equivalent weight of more than 5 KDa, preferably more than 10 KDa even more preferably more than 15 KDa.
Statement 19, composition properties. The composition of any of the preceding statements of properties wherein the preformed polymer and/or the in situ polymer product have a wt avg molecular weight of at least 5 kDa.
Statement 20, composition properties. The composition of any of the preceding statements of properties and/or of any of the succeeding statements of pretreatment comprising the reactive composition and/or the pretreatment composition wherein the one or more polymers in the first component and/or the in situ linking material in the second component and/or the pretreatment base compound and/or any combinations thereof have an average functional group equivalent weight of greater than or equal to their respective entanglement molecular weight.
Statement 21, composition properties. The composition of any of the preceding statements of properties and/or any of the succeeding statements of pretreatment comprising the preformed composition and/or the reactive composition and/or the pretreatment composition wherein the preformed polymer and/or the pretreatment base compound and/or the one or more polymers in the first component and/or the in situ linking material in the second component and/or combinations thereof contain non-reactive functional groups with multiple hydrogen bonding sites, an ionomer, reversible covalent bonds, or any combination thereof; wherein optionally the reversable covalent bonds are selected from Diels Alder, disulfide, esters, acid amides or sterically hindered ureas.
Statement 22, composition properties. The composition of any of the preceding statements of properties and/or any of the succeeding statements of pretreatment comprising the preformed composition and/or the reactive composition and/or the pretreatment composition wherein the preformed polymer and/or the one or more polymers in the first component and/or the in situ linking material in the second component and/or the pretreatment base compound and/or combinations thereof are selected such that they have a surface energy more than 40 mN m⁻¹.
Statement 23, composition properties. The composition of any of the preceding statements of properties or any of the succeeding statements of pretreatment wherein any one or more of the compositions have a surface energy of less than 24 mN m⁻¹.

### Dispersant

Statement 24, dispersant. The composition of any of the preceding statements of compositions and/or properties and/or any succeeding statements of pretreatment further comprising: a pigment dispersant, wherein the pigment dispersant can disperse a pigment in the medium and satisfy the dispersant compatibility requirement test.
Statement 25, dispersant. The composition of Statement 24 wherein the pigment dispersed with the pigment dispersant is not compatible with any layer from the pretreatment composition on the keratinous surface to enable better color removal.
Statement 26, dispersant. The composition of any of the preceding statements of dispersants wherein the pigment dispersed with the pigment dispersant is not compatible with the keratinous surface to enable better color removal.
Statement 27, dispersant. The composition of any of the preceding statements of dispersants wherein the pigment dispersed with the pigment dispersant is compatible with any pretreatment layer on the keratinous surface to enable better color remanence.
Statement 28, dispersant. The composition of any of the preceding statements of dispersants wherein the pigment dispersed with the pigment dispersant is compatible with the keratinous surface to enable better color remanence.
Statement 29, dispersant. The composition of any of the preceding statements of dispersants, wherein the pigment dispersed with the pigment dispersant is incompatible to slightly compatible with the keratinous surface to facilitate coating removal.

### Additives

Statement 30, additive. The composition of any of the preceding statements of composition, properties and/or dispersant further comprising: a microfibril present with any one or more of the first component, the second component and the pretreatment composition wherein the microfibril has a fiber length between 1 nanometer and 10 micrometers.
Statement 31, additive. The composition of any of the preceding statements of composition, properties and/or dispersant further comprising: a filler material present with any one or more of the first component, the second component and the pretreatment composition wherein the filler material is a non-chromatic material with a particle size between 2 nm and 500 nm.
Statement 32A, additive. The composition of any of the preceding statements of composition, properties and/or dispersant further comprising: a polyolefin as macromolecular strands or nanoparticles present in any one or more of the first component, the second component and the pretreatment composition wherein the nanoparticles are selected from the group consisting of smectites, kaolins, illites, chlorites, attapulgites and mixtures or inorganic metal oxides selected from the group consisting of silica, titanium oxide, zirconium oxide, aluminum oxide, magnesium oxide, boehmite alumina, hydrotalcite.
Statement 32B, additive. The composition of the preceding statements of composition, properties and/or dispersant and/or the pretreatment composition of the succeeding statements of pretreatment further comprising: a carbon nanotube, wherein the Carbon Nanotube is added to either the first, second component or mixtures thereof of the composition or to the base compound of the pretreatment composition.
Statement 32C, additive. The composition of the preceding statements of composition, properties and/or dispersant and/or the pretreatment composition of the succeeding statements of pretreatment further comprising: a nanofiller of graphite oxide mixed polymer, wherein the nanofiller of graphite oxide mixed polymer is added to either the first or second component or mixtures thereof or to the base compound of the pretreatment composition.
Statement 32D, additive. The composition of the preceding statements of composition, properties and/or dispersant and/or the pretreatment composition of the succeeding statements of pretreatment further comprising: a graphene additive wherein the graphene additive is added to either the first, second or component or mixtures thereof or to the base compound of the pretreatment composition.
Statement 33, additive. The composition any one of Statements 30-32A-D wherein at least one of the microfibril, filler material, polyolefin macromolecular strand, nanoparticles, carbon nanotubes, graphite oxide mixed polymer or graphene additive contains a functional group that forms a complementary reactive pair with either of a first or second functional group present in the composition.
Statement 34A, additive. The composition of any of the preceding statements of composition, properties and/or dispersant and/or the pretreatment composition of the succeeding statements of pretreatment further comprising one of more UV filters.
Statement 34B, additive. The composition of Statement 34A comprising at least one or more UV filters, wherein the UV Filter is selected and added at a level to provide UV protection to the pigment(s) selected within any one or more of the preformed composition, the reactive composition and/or the pretreatment composition or any combination thereof.
Statement 35, additive. The composition of any of the preceding statements of composition, properties and/or dispersant and/or the pretreatment composition of the succeeding statements of pretreatment further comprising one of more radical scavengers.
Statement 36, additive. The composition of any of the preceding statements of composition, properties and/or dispersant and/or the pretreatment composition of the succeeding statements of pretreatment further comprising one or more triplet formation inhibitors.
Statement 37A, additive. The composition of any of the preceding statement of composition, properties and/or dispersant and/or the pretreatment composition of the succeeding statements of pretreatment wherein at least one of the one or more pigments have been subjected to a prior cleaning process, to remove residual material from the pigment.
Statement 37B, additive The composition of the preceding statements of additives further comprising: a metal compound which can absorb or reflect UV light, wherein the metal compounds are selected from chromium, titanium, zinc, nickel, manganese, iron, niobium, silver, gold, aluminum, hafnium, tantalum and wherein the metal compound which absorb or reflect UV light is added to either the first, second or third component or mixtures thereof.
Statement 38, additive. The composition of any of the preceding statements of additives wherein at least one of the preformed composition, reactive composition and/or pretreatment composition and/or dispersant and/or any combination thereof have UV filtering properties.
Statement 39A, additive. The composition of any of the preceding statements of additives further comprising polyvinylidene fluoride for provision of UV protection and chemical resistance.
Statement 39B, additive. The composition of any of the preceding statements of additives wherein the pigment is pre-coated with polyvinylidene fluoride.
Statement 40, additive. The composition of the preceding statements of composition, properties, dispersant and/or additive and/or the pretreatment composition of the succeeding statements of pretreatment further comprising: an adherent active, wherein the adherent active adheres to the keratinous surface using one or more of the following; hydrogen-bond, ionic, covalent and physical/mechanical interactions and wherein the adherent active is added to either the first, second component or mixtures thereof or the base compound of the pretreatment composition.

### Pretreatment Composition

Statement 41, pretreatment. A pre-treatment composition for a keratinous surface comprising; a medium and a base compound wherein the base compound is selected to interact with the keratinous surface.
Statement 42, pretreatment. The pre-treatment composition of Statement 41 wherein the base compound is selected from a linear or branched polymer comprising linear polyethyleneimine, branched polyethylene imine, a copolymer of aminoethyl (meth)acrylate and ethyl (meth)acrylate, polyallylamine hydrochloride, polydiallyldimethyl ammonium chloride, polyvinylamine, (vinylamine-styrene) copolymer, poly(omega-aminoalkyl(meth)acrylate), polyvinylpyrrolidone, poly (2-vinyloxazoline), a dimethylsilicone with multiple pendant alkylamine groups and random or block copolymers thereof and mixtures thereof.
Statement 43, pretreatment. The pre-treatment composition of Statement 42 wherein base compound is a branched polyethylene imine with a weight average molecular weight in the range of about 400 Da to about 1 Mda.
Statement 44, pretreatment. The pre-treatment composition of Statement 42 wherein the base compound is selected from a dimethylsilicone with multiple pendant alkylamine groups and a first siloxane of the formula R₁-L-Si(OR₂)ₐ(R₃)_{b} wherein R₁ represents a first functional group, L is a linear or branched, divalent C₁-C₂₀ alkylene group and R₂ and R₃ are each independently a C₁-C₆ alkyl group and a is an integer from 1 to 3 and b is an integer equal to 3−a
or an oligomeric version thereof; or a second siloxane of the formula J[Si(OR₂)ₐ(R₃)_{b}]ₓ
Wherein J is a linking group which may contain multiple functional groups and is linked to x Si atoms and x is an integer of 2 to 6.
   Statement 45, pretreatment. The pre-treatment composition of any of statements 41-44 wherein the base compound comprises a nanoemulsion.
   Statement 46, pretreatment. The pre-treatment composition of any of statements 41-45 further comprises an additive comprising natural and or semi synthetic and or synthetic waxes or wax emulsions or a combination thereof.
   Statement 47, pretreatment. the pretreatment composition of statement 46 wherein the wax or wax emulsion has a melting point higher than 40° C., more preferably higher than 75° C., even more preferably higher than 110° C.
   Statement 48A, pretreatment. The pre-treatment composition of any of the preceding statements of pretreatment compositions wherein the base compound is selected from polymeric or prepolymeric and small molecule species
   Statement 48B, pretreatment. The pre-treatment composition of statement 47A wherein the base compound comprises first functional groups that are capable of ionization.
   Statement 48C, pretreatment. The pre-treatment composition of the preceding pretreatment statements wherein the base compound is a biofilm produced from a microorganism that in nature forms adhered biofilms on keratinous surfaces.
   Statement 49A, pretreatment. The pre-treatment composition of any of statements 41-48A-C further comprising an additive comprising a pro-wetting agent which can modify the surface energy of the keratinous surface.
   Statement 49B, pretreatment. The pre-treatment of Statement 49A wherein the pro-wetting agent increases the surface energy of the keratinous surface.
   Statement 49C, pretreatment. The pre-treatment of Statement 49A wherein the pro-wetting agent decreases the surface energy of the keratinous surface.
   Statement 50, pretreatment. The pre-treatment composition of any of statements 41-49C further comprising an additive comprising a preparative removal agent which can be subsequently used to facilitate the removal of any subsequent composition applied onto the keratinous surface.
   Statement 51, pretreatment. The pre-treatment composition of any of statements 41 to 50 wherein the weight percent of the base compound is 0.1-10 wt. % relative to the total composition.
   Statement 52, pretreatment. The pre-treatment composition of any of statements 41 to 51 further comprising a pigment.
   Statement 53A, pretreatment. The pre-treatment composition of statement 52 wherein the pigment is a colored organic microparticle.
   Statement 53 B, pretreatment. The pretreatment composition of statement 53A wherein the pigment is Pigment Yellow 83, Pigment Green 36, Pigment Blue 60, Pigment Blue 66, Pigment Blue 16, Pigment Black 6, Pigment Red 122, Pigment Red 5, Pigment Red 112, Pigment Violet 19, aluminum.
   Statement 54, pretreatment. The pretreatment composition of statement 52 wherein the pigment is a colored inorganic microparticle
   Statement 55, pretreatment. The pretreatment composition of statement 54 wherein the pigment is pigment White 6 or zinc oxide or barium sulfide.
   Statement 56, pretreatment. The pretreatment composition of statement 54 wherein the pigment is a metal microflake comprising aluminum.
   Statement 57, pretreatment. The pre-treatment composition of any of statements 52-56 comprising any combination of colored organic microparticles, inorganic microparticles and metal microflakes.
   Statement 58, pretreatment. A prior alteration composition comprising an oxidant, preferably hydrogen peroxide.
   Statement 59, pretreatment. The prior alteration composition of statement 58 further comprising a persulfate compound chosen from one or more of ammonium persulfate, potassium persulfate or sodium persulfate to facilitate bleaching of melanin with the hair to give the effect of lightening the appearance of the hair.
   Statement 60, pretreatment. A prior alteration composition of statement 59, pretreatment further comprising one or more of alkali materials, optionally where the alkali is optionally chosen from ammonia or salts or monoethanolamine at a level sufficient to activate the oxidant of Statement 58 and/or 59 to facilitate bleaching of melanin with the hair to give the effect of lightening the appearance of the hair.

### Topcoat

Statement 61, topcoat. A post-treatment composition comprising a medium and a topcoat material.
Statement 62, topcoat. The post-treatment composition of statement 61 wherein the topcoat material is one or more of a UV filter, a visible light filter, a radical scavenger, a triplet formation inhibitor, a water repellant, a hair spray formulation for holding a hair style or any combination thereof.
Statement 63, topcoat. The post-treatment composition of statement 61 wherein the topcoat material comprises a plurality of polymeric particles, formed of a polymeric material having neutralized acid moieties, wherein, in each of at least a portion of said polymeric particles, said polymeric material envelops at least one pigment particle; and, wherein said polymeric material having said neutralized acid moieties includes, mainly includes, consists essentially of, or consists of one or more neutralized copolymer selected from the group consisting of neutralized alkene-acrylic acid copolymer, neutralized alkene-methacrylic acid copolymer and neutralized aery lamide/acry late copolymer wherein the polymer is selected from:
a) neutralized ethylene-acrylic acid (EAA) copolymer.
b) neutralized ethylene-methacrylic acid (EMAA) copolymer.
c) neutralized arylamide/acrylate (AAA) copolymer.
   Statement 64, topcoat. The post treatment composition of statement 61 wherein the topcoat material is an aqueous dispersion comprising: a plurality of polymeric particles, formed of a hydrophilic polymeric material having neutralized acid moieties, wherein, in each of at least a portion of said polymeric particles, said hydrophilic polymeric material envelops at least one pigment core particle, said plurality of polymeric particles being dispersed within said aqueous dispersion; and the composition being adapted to produce a pigmented polymeric layer adhering to an external surface.
   Statement 65A, topcoat. The post-treatment composition of Statement 61 wherein the topcoat material comprises a reactive composition of the statements of composition and/or a pretreatment composition wherein the reactive composition comprises a first component and/or a second component with reactive complementary pairs of reactive functional groups as recited by the statements of composition and/or the pretreatment composition comprises a base compound, and further comprising any combination of any component of the reactive composition and/or the base compound wherein one or more of the components of the reactive composition and/or the pretreatment composition comprise a wear resistant olefinic polymer, a sacrificial non-sticky substance, a polyester, a poly (higher alkyl (meth)acrylate), a shellac, a volatile organic solvent, a fragrance and any combination thereof.
   Statement 65B, topcoat. The post-treatment composition of Statement 64 wherein at least one of the first and/or second components is selected from a polyester or a poly (higher alkyl (meth)acrylate)) having at least one of the members of the reactive functional group complementary pairs of isocyanate and alcohol; multicyclic carbonate and amine; carbodiimide and carboxylic acid; (meth)acryloyl, vinylcarbonyl or crotonoyl and amine or mercaptan; and aza and alkynyl.
   Statement 65C, topcoat. The post-treatment composition of statement 65A or B wherein the topcoat material further comprises a polymeric or prepolymeric species of the reactive composition having the other member of the complementary pair of reactive functional groups.
   Statement 65D, topcoat. The post-treatment composition of any of statements 65A-C wherein the first and/or second components further comprise non-reactive functional groups that have hydrogen bonding, and/or ionizing electrostatic bonding capability.
   Statement 65E, topcoat. The post-treatment composition of any of statements 65A-D wherein at least one of the first and second components of the reactive composition recited by composition statements 1-11 and/or composition properties statements 12-23 provides an excess of one of the reactive functional groups of the complementary pair and the excess is capable of interacting with a reactive functional group of the first or second component of the post treatment composition recited by any of statements 65A-D.

### Post Care Compositions

Statement 1, post care. A care composition for caring for a coated keratinous surface to enhance the coatings longevity comprising a medium and a surface care active for the coated keratinous surface.
Statement 2, post care. The care composition of Statement 1, post care, wherein the surface care active is a lubricating agent.
Statement 3, post care. The care composition of Statement 1 post care, wherein the surface care active is a sacrificial liquid layer.
Statement 4, post care. The care composition of Statement 1, post care, wherein the surface care active is a feel modifier.
Statement 5, post care. The care composition of Statement 1, post care wherein the surface care active comprises a reactive composition of the statements of composition.
Statement 6, post care. The composition of Statement 1-5, post care, which is a hair care composition formulated to deposit at least one surface care active which increases the color lastingness.
Statement 7, post care. The care composition of statement 1, post care, wherein the surface care active is a mild surfactant.
Statement 8, post care. The care composition of statement 1 post care, wherein the surface care active is a polymeric surfactant.
Statement 9, post care. The care composition of statement 1, post care, wherein the surface care active is a non-penetrating surfactant with a molecular volume larger than about 450 cc per mol.
Statement 10, post care. The care composition of statement 1, post care, wherein the surface care active is a non-ionic surfactant.
Statement 11, post care. The care composition of statement 1, post care, wherein the surface care active is a cationic surfactant.

### A Protective Composition

Statement 1, protective comp. A composition for treating skin or hair to form a protective easily removable film to prevent staining of skin/specific parts of the hair comprising a polymer or prepolymer, and optionally other formulation aids.
Statement 2, protective comp. The composition for treating skin or hair to form a protective easily removable film of statement 1, protective comp, wherein the materials are selected such that their whose polarity is opposite to that of the subsequently applied hair coloring composition (for example if the hair colorant is water based, the protective composition remaining on the skin is comprised of water-insoluble materials.

### Removal Composition

Statement 1, removal comp. A composition for removing a coating from the surface of a keratinous surface comprising a medium and a removal agent
Statement 2, removal comp. The composition of statement 1, removal comp, wherein the removal agent is an organic amine.
Statement 3, removal comp. The composition of statement 1, removal comp, wherein the removal agent is ammonium hydroxide or sodium fluoride.
Statement 4, removal comp. The composition of statement 1, removal comp, wherein the removal agent is a carbonate or ester cleavage agent.
Statement 5, removal comp. The composition of statement 1, removal comp, wherein the removal agent is wax and the composition is applied prior to or simultaneous with or combined with any one or more of the preformed composition, the reactive composition and/or the pretreatment composition.
Statement 6, removal comp. The composition of statement 1 removal comp, wherein the removal agent is a cellulose derivative and the composition is applied prior to or simultaneous with or combined with any one or more of the preformed composition, the reactive composition and/or the pretreatment composition.
Statement 7, removal comp. The composition of statements 1-6, removal comp, wherein the removal agent is present a level between 0.1 and 20%
Statement 8, removal comp. The composition of statements 1-7, removal comp, wherein the medium is aqueous or aqueous organic.
Statement 9, removal comp. The composition of statements 1-7, removal comp, wherein the medium is aqueous organic and the organic is a volatile alcohol, a ketone or an ester.

### Methods for Applying Compositions

Statement 1A, composition method. The method of application of any one or more of the compositions recited by the statements of compositions, of composition properties, of dispersants, of additives, comprising the step of applying any one or more of the compositions and any combination thereof to the surface of the keratinous surface and optionally allowing the any one or more of the compositions to remain on the keratinous surface for a specified period of time.
Statement 1B, composition method. The method of statement 1A, composition method, wherein the one or more compositions are allowed to remain on the keratinous surface for a period of time of at least two minutes before the method of statement 2, composition coating, is applied.
Statement 2, composition method. The method of application according to statement 1, composition method, further comprising rinsing the keratinous surface with a medium to remove at least a portion of applied composition prior to or following the method of statement 3, composition method and/or prior to or following the method of statement 4, composition method.
Statement 3, composition method. The method of statement 1 or 2, composition method, wherein after the one or more of the compositions recited by the statements of compositions, of composition properties, of dispersants, of additives and any combination thereof are applied to the keratinous surface, performing an additional step comprising drying the one or more compositions on the keratinous surface at a temperature greater than 40 C.
Statement 4, composition method. The method of any of statements 1-3, composition method wherein after the one or more compositions are applied to the keratinous surface, performing an additional step comprising curing the one or more compositions with a device which provides UV light, or kinetic energy providing an increased temperature or electromagnetic energy, or any combination thereof.
Statement 5, composition method. The method of statement 2, composition method, wherein after the one or more compositions are applied to the keratinous surface and before the one or more compositions are dried and/or cured according to statement 3 and/or statement 4, composition method, at least a portion of the keratinous surface with one or more compositions is rinsed with a medium that will remove the one or more compositions to provide at least a portion of keratinous surface without the one or more compositions.

### Cleaning

Statement 1, cleaning. A method for use prior to practice of the methods recited by the statements of composition coatings comprising first applying a cleaning composition to the keratinous surface and then rinsing the keratinous surface to prepare the keratin surface for the application according to the composition methods to enable better adhesion of the keratin surface.
Statement 2, cleaning. The method of Statement 1, cleaning further comprising during the step of applying a cleaning composition and the rinsing the keratinous surface using mechanical or energy based cleaning.
Statement 3, cleaning. The method of Statement 2, cleaning wherein the mechanical or energy based cleaning is provided by moving a microfiber material over the keratinous surface to produce a cleaner keratinous surface.
Statement 4, cleaning. The method of Statement 2 cleaning wherein the mechanical or energy based cleaning is provided by using an ultrasound device on the keratinous surface to produce a cleaner keratinous surface.
Statement 5, cleaning. The method of Statement 2, cleaning wherein the mechanical or energy based cleaning is provided by using a device using electromagnetic frequencies on the keratinous surface to produce a cleaner keratinous surface.

### Pretreatment Method

Statement 1, pretreating. A method of pretreatment comprising, prior to application of any of the methods recited by the statements of composition method, applying any one or more of the pretreatment compositions recited by any of the pretreatment statements to the keratinous surface, optionally leaving for a period of time, optionally rinsing and optionally drying the keratinous surface wherein the pre-treatment composition changes the keratinous surface.
Statement 2, pretreating. The method of pretreatment of statement 1, pretreating, comprising applying any one or more of the cleaning methods of cleaning statements 1-5 before the method of pretreatment of statement 1, pretreatment.
Statement 3, pretreating. The method of any of statements 1 or 2, pretreating, further comprising sequentially applying two pretreatment compositions of any of the pretreatment compositions recited by the statements of pretreatment compositions wherein the two pretreatment compositions differ.
Statement 4, pretreating. The method of statement 3, pretreating, further comprising rinsing and drying after application of one or more of the applications of two different pretreatment compositions.
Statement 4, pretreating. The method of statement 3, pretreating, wherein the differing first and second pretreatment compositions are applied to separate portions of keratinous surface and each portion of keratinous surface with applied pretreatment composition is optionally allowed to rest for a period of time, optionally rinsed following the period of time and optionally dried.
Statement 5, pretreating. The method of Statement 4, pretreating, wherein where the difference in surface energy of the first and second portions of the keratinous material is reduced after treatment.
Statement 6, pretreating. The method of Statement 4, pretreating, wherein where the difference in surface energy of the first and second portions of the keratinous material is increased after treatment.
Statement 7, pretreating. The method of statement 4, pretreating, wherein the difference in zeta potential between the first and second portions of the keratinous surface is reduced.
Statement 8, pretreating. The method of statement 2, pretreating, further comprising following any of the cleaning methods recited by statements 1-5, cleaning and before practice of the pretreatment method recited by statement 1, pretreatment, the step of increasing the surface area and increasing adhesion of the pretreatment composition by applying a crinkle devise to the keratinous surface.
Statement 9, pretreating. The method of statement 8, pretreating, wherein the step of increasing surface area and adhesion is accomplished by passing the keratinous surface through crinkle rollers which have micron sized bumps on them, wherein the bump diameter is preferably between 1 and 50 microns and the bump material is a material with a hardness on Mohs' scale higher than 2.
Statement 10, pretreating. The method of pretreatment comprising following any of the methods for cleaning recited by cleaning statements 1-5, and before application of any of the pretreatment methods recited by pretreating methods 1-7, the step of: moving an atmospheric plasma generation device into a location proximate to a first portion of the keratinous surface, then for a period of time treating a first portion of the keratinous surface with the atmospheric plasma generation device, then removing the atmospheric plasma generation device from the location proximate to the first portion of the keratinous surface.

### Topcoat Method

Statement 1, topcoat method. The method of topcoating comprising applying a topcoat composition recited by any of topcoat composition statements 61-65E to a keratinous surface treated by any one or more of the composition and pretreatment methods recited by composition coating statements 1-5 and pretreatment methods recited by pretreatment method statements 1-10.
Statement 2, topcoat method. The method of topcoat method statement 1 wherein the topcoat composition is recited by topcoat composition statement 62.
Statement 3, topcoat method. The method of statement 2 topcoat method, wherein the components of a UV filter a visible light filter and/or a radical scavenger and/or combinations thereof are present at higher concentrations than the UV protection afforded by the compositions of Statements 1-60.
Statement 4, topcoat method. The method of any of statements 1-3 topcoat method, wherein the method includes the steps of: application of a pre-treatment composition of statement 48B, and application of a post-treatment composition of statement 65C wherein the first functional group is of opposite charge to the second functional group
Statement 5, topcoat method. The method of any of statements 1-3, topcoat method, comprising at least the steps of pre-treatment, composition and post treatment wherein the wash and mechanical resistance of the topcoat applied according to the method recited by topcoat method statement 1 is higher than when the same topcoat is applied singly to keratinous surface.

### Care Composition Method

Statement 1, care method. The method recited by any of the statements of composition method, cleaning method, pretreatment method, topcoat method, further comprising a step of maintaining the coating on a keratinous surface in the days and weeks after its application to keratinous surface by applying any one or more care compositions recited by the statements 1-11, post care, and optionally rinsing and optionally drying.
Statement 2, care method. The method of Statement 1, care method, wherein the coated keratinous surface is colored hair which further uses computer-assisted determination to select which hair care compositions of statements 1-11 post care to use, comprising the following steps:
providing at least one parameter of the hair prior to coloring to a data processing apparatus then;
providing at least one parameter of the colored coating on the hair surface to the data processing apparatus;
optionally providing associated geolocation data to the data processing apparatus determining at least one hair care composition which can maintain the hair color from data stored either directly or remotely to the data processing apparatus;
presenting the recommended product or products to the user; and
thereby rendering the user able to separately and at any time apply the recommended product to the colored hair.

### Method for Removal

Statement 1, removal. The method of at least partially removing from the keratinous surface any one or more of the dried and optionally cured compositions applied to the keratinous surface according to the statements of composition method, pretreatment methods, topcoat methods, care methods, comprising applying a removal composition recited by any one or more of statements 1-9 removal comp to the keratinous surface with one or more dried and optionally cured composition wherein the removal leaves the keratinous surface substantially free from any keratinous damage.
Statement 2, removal. The method of Statement 1, removal, wherein the removal occurs within two hours of any of the steps of one or more of the methods recited by the statements of composition method, pretreatment method, topcoat method and/or care method.
Statement 3, removal. The method of Statement 1, removal, wherein the removal occurs after more than 12 hours of any of the steps of the composition method, pretreatment, topcoat and/or cares.
Statement 4, removal. The method of removal of any of statements of removal 1-3 wherein the removal composition recited by any of statements 1-9 removal comp, is applied on the keratinous surface for a period of time between 30 seconds and 2 hours, more preferably between 1 minute and 60 minutes, even more preferably between 2 minutes and 45 minutes.
Statement 5, removal. The method of removal of any of statements of removal 1-4 wherein the removal of the coating on the keratin surface is further assisted through using mechanical or energy based cleaning.
Statement 6, removal. The method of statement 5 removal wherein the mechanical or energy based cleaning is provided by moving a microfiber material over the keratinous surface to produce a cleaner keratinous surface.
Statement 7, removal. The method of statement 5, removal, wherein the mechanical or energy based cleaning is provided by using an ultrasound device on the keratinous surface to produce a cleaner keratinous surface.
Statement 8, removal. The method of statement 5 removal, wherein the mechanical or energy based cleaning is provided by using a device using electromagnetic frequencies/light or heat on the keratinous surface to produce a cleaner keratinous surface.
Statement 9, removal. The method of any of removal statements 1-8 further comprising the step of passing the surface of the keratinous surface through an exfoliating sheet.
Statement 10, removal. The method of any of the preceding removal statements 1-9 further comprising the step of passing the surface of the keratinous surface through an exfoliating brush.
Statement 11, removal. The method of any of the preceding removal statements 1-10 comprising optionally filtering and bagging the debris removed from the hair to prevent the debris from entering the environment.
Statement 12, removal. The method of removal statements 1-11 wherein at least one pre-treatment composition is a pre-treatment composition including a wax addition recited by pretreatment statement 46 or 47 and the removing step involves application of heat.

### Universal Statements of Preferred Keratinous Surface

Statement 1, PREFERENCE. A composition of any statement of composition, properties, additives, pretreatment, topcoat, dispersant, care or any combination thereof; or a method of any statement of a composition coating, cleaning, pretreatment, topcoat, care or removal or any combination thereof, wherein the keratinous surface is keratin fiber, preferably hair, more preferably human hair.

### Methods of Coloration

Statement 1, coloration. A method of coloration of human root hair which has not been previously treated, comprising the steps of selecting any one or more compositions, or any combination thereof, of the compositions with color of statements 1-65E to change the color of the root hair and applying the composition to the root hair to form a coating thereon, wherein the coloring of the root hair does not lead to any damage of the root hair
Statement 1A, coloration. A method of statement 1, coloration, further comprising removal of the coating by any one or more of the methods for removal recited by removal statements 1-12 without any damage to the root hair.
Statement 2, coloration. The method of statement 1, coloration, wherein the human hair has been previously colored on the non-root hair wherein the application of the composition with color is only performed only on the root hair, thereby removing the need to remove the color from the previously color hair.
Statement 3, coloration. The method of statement 1 or 2, coloration, wherein the application of the composition with color to the root hair is performed using a root hair application device.
Statement 4, coloration. The method of any of statements 1-3, coloration, wherein the method involves a step of selecting the composition color such that it matches the previously colored hair wherein the coloring composition is selected such that the resulting hair color is within De76 of 6 of the previously colored hair.
Statement 5, coloration. A method of coloration of human non root hair comprising the steps of selecting any one or more compositions, or any combination thereof, of the compositions with color of statements 1-65E to change the color of the non root hair, and applying the composition to the non-root hair to form a coating thereon wherein the coloring on the non root hair does not lead to any damage of the non root hair and enables the user to return the color of their non-root hair back to the color of the root hair.
Statement 6, coloration. The method of statement 5, coloration, wherein the hair is previously colored hair and the application is only performed only on the non root hair removing the need to use bleaching chemistry on the previously colored hair.
Statement 7, coloration. The method of statement 5 or 6, coloration, wherein the application of the coloring composition to the non root hair is performed using a non root hair application device.
Statement 8, coloration. The method of any of statements 5-7, coloration, wherein the method involves a step of selecting the coloring composition such that it matches the root hair color wherein the method involves selecting the coloring composition such that the resulting hair color is within De76 of 6 of the persons root hair.
Statement 9, coloration. A method for coloring hair with a coating from any one or more compositions, or any combination thereof, of the compositions with color of statements 1-65E comprising the step of applying a pre-treatment composition of any of statements 41-60 containing an additive which forms a thermally active layer on the hair wherein the thermally active layer has a transition temperature above body temperature and has the ability to flow above the transition temperature.
Statement 9A, coloration. A method of statement 9, coloration, further comprising the step of removing the colored coating by applying heat to the hair when the coloring composition is to be removed from the hair.
Statement 10, coloration. A method for coloring hair with a coating from any one or more compositions, or any combination thereof, of the compositions with color of statements 1-65E comprising the step of application of a pre-treatment composition of any of statements 41-60 containing an additive which forms a thermally active layer on the hair where the thermally active layer has a transition below room temperature which enables removal of the thermally active layer below the transition temperature.
Statement 10A, coloration. A method of statement 10, coloration, further comprising removing the coating by applying cooling to the hair when the coating is to be removed from the keratinous surface.
Statement 11. The method for coloring hair with a coating from any one or more compositions, or any combination thereof, of the compositions with color of statements 1-65E comprising the step of application of a pre-treatment composition of any of statements 41-60 containing an additive which forms a thermally active layer on the hair wherein the thermally active layer has a transition above body temperature which has a Young's Modulus which enables hair styling effects below this transition temperature.
Statement 12, coloration. The method for coloring hair with a coating from any one or more compositions, or any combination thereof, of the compositions with color of statements 1-65E comprising the step of application of a pre-treatment composition of any of statements 41-60 containing an additive which forms a thermally active layer on the hair wherein the thermally active layer has a transition above body temperature which has a Young's Modulus which does not enable hair styling effects above this transition temperature.
Statement 13, coloration. The method for coloring hair with a coating from any one or more compositions, or any combination thereof, of the compositions with color of statements 1-65E comprising wherein at least one composition comprises a reactive composition of statement 1, composition wherein the first and second components of the reactive composition have first and second functional groups which are complimentary reactive pairs which can form covalent linkages in-situ to produce a long lasting color.
Statement 14, coloration. The method for coloring hair with a coating from any one or more compositions, or any combination thereof, of the compositions with color of statements 1-65E comprising wherein at least one composition comprises a reactive composition of statement 1, composition wherein the first and second components of the reactive composition have a functional equivalent molecular weight equal to or greater than the entanglement molecular weight.
Statement 15, coloration. The method of any of the preceding coloration statements 1-14 wherein the method further comprises an initial color preview step wherein the hair color application is performed on a section of a user's hair, and after application the resulting hair color is evaluated by a color assessment step relative to the user's desired look, and if the section color does not meet the desired look, a method for removal of any of statements 1-12, removal, is applied to remove the hair color, or if the color is desired, the hair color application step is then applied to color the rest of the users hair.
Statement 16. The method of statement 15, coloration, wherein the initial color preview step further comprises using a plurality of different hair coloring compositions each applied to different sections of the user's hair and the color assessment step evaluates the plurality of different colors on different hair sections and selects one of the plurality of colors; the color removal step is performed on the rejected colors, and the selected color is applied to the rest of the user's hair.
Statement 17. The method of statement 16, coloration, wherein the method does not involve the final application of the color composition to the rest of the hair, and the selected color is removed in the color removal step to show the user that the color can be removed.
Statement 18. The method of statements 15-17, coloration, wherein the drying and/or curing step of the coloring composition is not allowed to go to completion to enable easier removal of the color from the section of hair.

### Method for Applying Protective Layer

Statement 1, protection. A method of any of the preceding statements of coloration further comprising the step of applying the composition for treating skin or hair to form a protective, easily removable film of Statement 1 and/or 2, protective comp, wherein a protective easily removable film is formed to prevent staining/specific parts of the hair during a subsequent hair coloring procedure, and wherein the film is resistant to subsequently applied hair coloring compositions such that surfaces below said film exhibit a color change post coloring of less than 1 dE according to the CIE L*a*b* color system.
Statement 2, protection. The method of statement 1, protection, comprising the use of a device or mechanism which, when pressed in contact with skin or hair for a period of time transfers a protective easily removable film onto the skin or hair.
Statement 2A, protection. The method of statement 2, protection wherein the period of time is less than 5 minutes.
Statement 3, protection. The method of statement 1, protection, further comprising the step of removing the protective, easily removable film after said hair coloring treatment.
Statement 3A, protection. The method of statement 2, protection wherein the removal is accomplished by mechanical peeling.

### Color Selection

Statement 1, color selection. A method for use with any one or more of the method statements for the composition method, the pretreatment method, the topcoat method, the care method, the coloration method and/or the protection method, wherein the compositions and hair colors thereof are selected using the following steps:
making an assessment of the condition of the user's hair
making an assessment of the users desired end hair color
using the information on the condition of the hair and the desired color to design and prepare an admixture of different pro-compositions to deliver the desired end color and performance on the user's hair.
   Statement 2, color selection. The method of statement 2, color selection, wherein the admixture is designed through use of an automated system which calculated the levels of the different pro-composition to deliver the desired end color.
   Statement 3, color selection. The method of any of statements 1 and/or 2, color selection, wherein the admixture is dispensed by the automated system.
   Statement 4, color selection. The method of any of statements 1-3, color selection, wherein the desired end color is created on hair which has been previously colored.
   Statement 5, color selection. The method of any of statements 1-4, color selection, wherein the combination of pro-compositions is designed to obtain a coloration that attains maxima for color washfastness, shine, shine retention, smooth/soft/silky hair feel, based on the analysis of the starting hair condition.
   Statement 6, color selection. The method of any of statements 1-5, color selection, wherein a fourth pre-treatment is performed and the fourth pretreatment applies an oxidative composition to the hair to pre-lighten or pre-color the hair or to activate the surface of the hair to enable a wide range of colors to be achieved with a subsequent coloring composition.
   Statement 7, color selection. The method of any of statements 1-6, color selection, further comprising selecting and creating a composition for a specific users characterized by the steps of using a computer-assisted determination of a hair coloring composition which produces a composition optimum for the wearer based on their location and use of data associated with the location such as but not limited to weather patterns, water hardness, pollution indices etc, comprising:
a) a data processing apparatus;
b) providing at least one parameter of the hair prior to coloring to the data processing apparatus;
c) providing at least one parameter of the multicomponent in situ linkable composition for coloring hair to the data processing apparatus;
d) providing associated geolocation data to the data processing apparatus
e) determining at least one hair color composition optimised for location from data stored either directly or remotely to the data processing apparatus
f) optionally including additives such as UV protectants, plasticizers, moisturizers based on geolocation associated parameters
g) presenting the recommended product or products to the user.
   Statement 8, color selection. The method of any of statements 1-7, color selection, further comprising selecting and creating a composition for a specific users characterized by the steps of: selecting a hair color with a user interface; receiving, from the user interface, an input of at least one base color intensity for the hair color; calculating a target amount for each pro-composition based on the plurality of base color intensities; and dispensing the calculated target amount of each pro-composition and additional formulations to form a composite hair color coating hair formulation, wherein the composite hair color coating formulation corresponds to the selected hair color.
   Statement 9, color selection. The method of any of statements 1-8, color selection, further comprising selecting and creating a composition for a specific users characterized by the steps of a) determining at least one keratinous surfaces property by means of at least one analytical method on a plurality of keratinous surfaces with differing keratinous conditions; b) assess by means of spectroscopy using one of more different wavelengths and or image acquisition using one of more different illuminants on the same plurality of keratinous surfaces; c) creating a calibration model, and/or a correlation model between the at least one analytical method and the at least one spectroscopy or image acquisition for the plurality of keratinous surfaces; d) receiving at least one spectroscopy or image acquisition of the keratinous surface of an individual; e) determining a keratinous condition of said individuals hair on the basis of the calibration and or correlation model; and f) output an individual composition for the treatment to the individuals keratinous surfaces in response to the determined keratinous condition
   Statement 10, color selection. The method of any of statements 1-9, color selection further comprising selecting and creating a composition for a specific users characterized by the steps of: selecting a hair color with a user interface; receiving, from the user interface, an input of at least one base color intensity for the hair color; calculating a target amount for each base color formulation based on the plurality of base color intensities; and dispensing the calculated target amount of each base color formulation and additional formulations to form a composite hair color multicomponent in situ linkable composition containing at least one pigment, wherein the composite hair color multicomponent in situ linkable composition containing at least one pigment corresponds to the selected hair color
   Statement 11, color selection. A method of any of statements 1-10, color selection, further comprising tracking hair stylists or colorant users arm movement to provide technique feedback during or after application or removal of the composition.
   Statement 12, color selection. The method of any of statements 1-11, color selection, wherein the hair stylist or user wears a motion/position tracking device and/or while using a hair salon implement comprising one or more of a brush, comb, hair dryer or application device is fitted with a tracking device.
   Statement 13, color selection. The method of statement 12, color selection, comprising producing technique training based on the output of a motion/position tracking device.
   Statement 14, color selection. The method of any of statement 12 or 13, color selection, comprising ensuring optimum application times, temperatures, combing forces, product spreading speed, and product amount per application, using the output of a motion/position tracking device.
   Statement 15, color selection. The method of any of statements 1-14, color selection, further comprising prior to use of any one or more of the application methods recited by the statements of the composition method, pretreatment method, topcoat method, care method, the step of:
application of a test amount of a representative composition of any one or more of the compositions of statements 1-65E to a portion of skin and leaving the composition on the skin for at least 5 minutes, preferable at least 1 hour, more preferably at least 12 hours and observing the skin for irritation and if the skin shows no sign of irritation continuing with the one or more application methods.

### Color Coating (Film) Properties

Statement 1, coating. A composition for treating a keratinous surface which results in the formation of a color coating of elastomeric material on the keratinous surface wherein the composition comprises any one or more of the compositions recited by statements 1-11, compositions, statements 12-23, properties, statements 24-29, dispersants, statements 30-40, additives, statements 41-60, pretreatment, statements 61-65E, topcoat and/or statements 1-11, post care wherein the compositions recited by the properties statements, dispersant statements and additive statements are adapted to be combined with one or more of the compositions recited by the statements of compositions, pretreatment and/or topcoat, and the compositions recited by the statements of compositions, pretreatment and topcoat are optionally adapted to be serially applied.
Statement 2, coating. A composition according to statement 1, coating, comprising combining any one or more of the compositions recited by the statements 1-60 of compositions, properties, additives and dispersant.
Statement 3, coating. A composition according to statement 1, coating, comprising combining any one or more of the compositions recited by statements 61-65E of pretreatment, and statements 24-60 of dispersant and additives.
Statement 4, coating. A composition according to statement 1, coating, comprising serially applying the composition of statement 2, coating, and the composition of statement 3, coating.
Statement 5, coating. A composition according to statement 1, 2 or 4, coating, wherein the composition recited by statement 1, compositions, is a preformed composition.
Statement 6, coating. A composition according to statement 1, 2 or 4, coating, wherein the composition recited by statement 1, compositions, is a reactive composition.
Statement 7, coating. A composition of statement 1, 2, 4 or 6 wherein the composition recited by statement 1, compositions, comprises:
a first component comprising a polymer having first reactive functional group;
a second component comprising an in situ linking material having second reactive functional groups;
a pigment in one or more of the first and second components, and
a medium in one or more of the first and second components;
wherein the first and second reactive functional groups are complementary reactive pairs and can form covalent linkages in situ and the first and second components are adapted to produce the color coating of a polymer product when applied to the keratinous surface, the medium is removed and the complementary reactive pairs cured.
   Statement 8, coating. The composition for treating a keratinous surface of statement 7, coating further comprising a pretreatment composition of any one of statements 41-60, pretreatment, comprising a medium and a base compound comprising an organic polymer or silicone polymer or organosilicone polymer having multiple amine groups wherein the pretreatment composition is adapted to produce an underlayer of the base compound on the keratinous surface when dried, the underlayer being adapted to be overlaid by one or more layers of the color coating of polymer product, and the underlayer and the color coating of polymer product being the elastomeric color coating.
   Statement 9, coating. A colored coating on keratin fibers formed from the composition of statement 7 or 8, coating, wherein the colored coating of elastomeric material has qualities of elastomeric stretch, tensile strength, flexibility, non-tackiness, thickness, tactile and aural sensations similar to the same qualities of untreated keratin fibers.
   Statement 10, coating. A colored coating on keratin fibers according to statement 9, coating wherein the qualities are assessed by tests of swatches of the colored coating on keratin fibers relative to a standard swatch of untreated keratin fibers.
   Statement 11, coating. A colored coating according to statement 9 or 10, coating, wherein the keratin fibers are human hair.
   Statement 12, coating. A colored coating according to statement 10, coating, wherein the test swatches and standard swatch are composed of natural, untreated human hair of approximately the same age.
   Statement 13, coating. The composition of Statements 1-12, coating, wherein the at least one pigment is selected such that the coefficients of thermal expansion of the pigment is with 20% of the elastomer material within which it is held.
   Statement 14, coating. The composition of Statements 1-13 wherein the elastomer material has additional ionic or hydrogen bonding functional groups.
   Statement 15, coating. The composition of Statements 1-14 wherein the elastomeric material has an ultimate elongation of at least 30%, more preferably at least 50%, even more preferably at least 70%, most preferably at least 100%.
   Statement 16, coating. The composition of Statements 1-15 wherein the elastomeric material has an ultimate compression of at least 30%, more preferably at least 40%, even more preferably at least 60%.
   Statement 17, coating. The composition of Statements 1-16 wherein the elastomeric material has a Young's Modulus of at least 1 MPa, more preferably at least 10 MPa, even more preferably at least 50 MPa or a Young's Modulus of less than 5 GPa, more preferably less than 2 GPa, even more preferably less than 1 GPa.
   Statement 18, coating. The composition of Statements 1-17 wherein the elastomer material adheres to and forms a continuous coating on the keratinous surface to anchor the elastomer material to the keratinous surface.
   Statement 19, coating. The composition of Statement 1-18 further wherein the elastomeric material has an average repeated elongation before failure at 60% of ultimate elongation of at least 250 cycles, more preferably 500 cycles, even more preferably at least 1000 cycles.
   Statement 20, coating. The composition of Statements 1-19 further wherein the elastomeric material has a surface energy less than 24 nN m⁻¹.
   Statement 21, coating The composition of Statements 1-20 further wherein the elastomeric material has surface energy components of ad and 6p such that
σ^{d} is between 18 and 36 mN m⁻¹
σ^{p} is between 0 and 25 mN m⁻¹
   Statement 22, coating. The composition of Statements 1-21 further wherein the elastomeric material has Hansen Solubility Parameters, δD, δP, δH that satisfy the equation:

$$\sqrt{4 * {( δ D - x )}^{2} + {( δ P - y )}^{2} + {( δ H - z )}^{2}} > 4$$

wherein 16<x<17.2, 0.8<y<6.7 and 1.2<z<13
   Statement 23, coating. The composition of Statements 1-22 further wherein the elastomeric material has Hansen Solubility Parameters, δD, δP, δH that satisfy the equation:

$$\sqrt{4 * {( δ D - x )}^{2} + {( δ P - y )}^{2} + {( δ H - z )}^{2}} < 4$$

wherein 16<x<17.2, 0.8<y<6.7 and 1.2<z<13
   Statement 24, coating. An elastomeric material comprised of at least two layers on a keratinous surface produced by sequential application to the keratinous surface of any two or more of the compositions recited by statements 1-11, compositions; statements 41-60, pretreatment;
statements 61-65E, topcoat; and statements 1-11 post care, wherein the compositions of statements 1-11 optionally further comprise properties, dispersants and additives of statements 12-40 and the pretreatments of statements 41-60 optionally further comprise dispersants and additives of statements 24-40, wherein:
one layer has a surface energy in the range between 12 and 22 mN m⁻¹ to repel sebum and, another layer has a surface energy in the range between 23 and 50 mN m⁻¹ to retain the sebum
   Statement 25A, coating. An elastomeric material according to statement 24, coating, wherein the compositions are selected from those of statements 1-11, compositions and statements 41-60, pretreatment.
   Statement 25B, coating. An elastomeric material according to statement 24, coating wherein the compositions are selected from those of statements 1-11, compositions and statements 61-65E, topcoat.
   Statement 25C, coating. An elastomeric material according to statement 24 wherein the compositions are selected from those of statements 1-11, compositions, statements 41-60, pretreatment and statements 61-65E, topcoat.
   Statement 25D, coating. An elastomeric material according to any of statements 25A-C, coatings wherein the compositions of statements 1-11, compositions and statements 41-60 pretreatment include the optional properties, additives and dispersants of statement 24, coating.
   Statement 26, coating. The composition of statements 1-25C wherein the elastomer material has a higher concentration of UV filter present on or near the surface, so that curing of the composition producing the elastomer is not unacceptably disrupted by presence of the UV filter.
   Statement 27, coating. The composition of statements 1-26 wherein the formed elastomeric material on the keratinous surface has a keratin coverage index greater than 50%, more preferably greater than 75%, even more preferably greater than 85%.
   Statement 28, coating. The composition of any of statements 1-27 wherein the elastomeric material on the keratinous surface has an average thickness between 50 nm and 4000 nm, more preferably between 100 nm and 3000 nm even more preferably between 250 nm and 2000 nm.
   Statement 29, coating. The composition of any of statements 1-28 wherein the elastomeric material on the keratinous surface has a color variation factor greater than 1.2.
   Statement 30, coating. The composition of any of statements 1-29 further wherein the elastomeric material on the keratinous surface has a surface contact angle with n-decane as a probe liquid of more than 450 degrees, more preferably more than 60°, even more preferably more than 90°.
   Statement 31, coating. The composition of any of statements 1-30 wherein the elastomeric material on the keratinous surface has a surface energy less than 20 mN m⁻¹, more preferably less than 19 mN m⁻¹, even more preferably less than 18 mN m⁻¹.
   Statement 32, coating. The composition of any of statements 1-31 wherein the elastomeric material on the keratinous surface has a surface contact angle with water as a probe liquid of less than 600 degrees, more preferably less than 40°, even more preferably less than 20°.
   Statement 33, coating. The composition of any of statements 1-32 wherein the elastomeric material on the keratinous surface has a hydrophilic photocatalytic surface.
   Statement 34, coating. The composition of any of statements 1-33 wherein the elastomeric material on the keratinous surface has a surface energy more than 5 mN m⁻¹, more preferably more than 10 mN m⁻¹, even more preferably more than 20 mN m⁻¹.
   Statement 35, coating. The composition of any of statements 1-34 further wherein the elastomeric material on the keratinous surface has a specific surface area more than 0.003 m² g⁻¹ more preferably more than 0.004 m² g⁻¹, even more preferably more than 0.005 m² g⁻¹.
   Statement 36, coating. The composition of any of statements 1-35 wherein the elastomeric material on the keratinous surface has at least one additive which is a catalyst which accelerates the decomposition of human sebum.
   Statement 37, coating. The composition of any of statements 1-36 wherein the elastomeric material on the keratinous surface which is resistant to sebum wherein the elastomeric material on the keratinous surfaces is oleophobic.
   Statement 38, coating. The composition of any of statements 1-37 wherein the elastomeric material on the keratinous surface which is resistant to washing wherein the pigmented elastomer on the keratinous surfaces is oleophobic.
   Statement 39, coating. The composition of any of statements 1-38 wherein the resulting elastomeric material is a microscopically inhomogenous and/or discontinuous film.

### Kits

Statement 1, kit. A kit for coating a keratinous surface comprising separate compartments in which the compositions of any of statements 1-65E are separately contained such that the kit provides a suitable shelf life for the composition and the kit is in a condition ready for use.
Statement 2, kit. A kit according to statement 1, kit further comprising one or more separate compartments for activators, UV devices, brushes, applicators, hair strand separators, hair segment separators, foil covering, microfiber blotters, sponges for storing, applying, drying, curing the compositions of statements 1-65E.
Statement 3, kit. The kit of statement 1 or 2, kit, further comprising in a separate compartment at least one post care composition of Statements 1-11, post care.
Statement 4, kit. The kit of statements 1-3, kit, further comprising in a separate compartment a removal composition of statements 1-9, removal, to enable the damage free removal of the coating from the keratinous surface.
Statement 5, kit. The kit of statements 1-4, kit, wherein the separate compartments are selected to ensure that the compositions of the compartments are stable for the user.
Statement 6, kit. The kit of statements 1-5, kit, wherein one of the compartments is oversized, so as to accommodate additional compositions contained within different compartments, facilitate mixing and application to the keratinous surface.
Statement 7, kit. The kit of statements 1-6, kit, further containing a tool to apply the compositions to the keratinous surface.
Statement 8, kit. The kit of statements 1-7, kit, further containing a set of written instructions with optional pictorial representations describing which composition to apply on which portion of a keratinous surface and the timing therefor.
Statement 9. The kit of statements 1-8, kit, which contains in separate compartments:
a first container with a first pretreatment composition of any of statements 41-60 to treat a first portion of the hair, and
a second container with a second pretreatment composition of any of statements 41-60 to treat a second portion of the hair,
where the first and second pretreatment compositions are different and
instructions on which pre-treatment to apply on which portion of the hair,
to enable a more consistent coating of the hair from root to tip and or from section to section.
   Statement 10, kit. The kit of statements 1-9, kit, for coloring root hair wherein the compositions are selected such that the obtain color result on the root hair that is within De76 of 4 of the color result displayed on an image printed on the kit instructions to facilitate selecting which product to choose to match the root hair to the color on the hair mid-lengths, optionally wherein the compositions provide no oxidative damage to the root hair, thereby improving the hair health versus oxidative color treatments, and optionally wherein the color can also be removed without damage.
   Statement 11, kit. The kit statements 1-8 and 10, kit, for coloring root hair wherein the compositions are selected to be applicable to root hair, and optionally provide inferior performance on non-root hair, thereby reducing the need for accurate placement on the roots by the user.
   Statement 11, kit. The kit of statements 1-8 and statements 10 and 11, kit, wherein the amount of coloring composition is sufficient to treat the root hairs of a person but significantly less than the amount used to treat a whole head of head of a typical person, preferably the amount of the coloring composition is between 5 and 100 ml, more preferably between 10 and 80 ml, even more preferably between 15 and 60 ml and optionally the kit is configured to enable easy postal delivery to a user at home.
   Statement 12, kit. The kit of statements 1-11, kit, wherein the kit further comprises an applicator specifically designed to enable placement of the compositions on root hair.
   Statement 13, kit. The kit of statements 1-8, kit, for coloring mid-length and tip hair wherein the compositions are selected such that they work on mid-length and tip hair to enable a user to color their hair back to their natural color, without causing oxidative damage.
   Statement 14, kit. The kit of statement 13, kit, wherein the amount of product is tailored for the amount of hair being treated away from the roots.
   Statement 15, kit. The kit of statement 14 wherein the amount of hair color composition is between 40 and 120 ml, more preferably between 50 and 100 ml.
   Statement 16, kit. The kit of statements 1-15, kit, which further comprises a device to assist with the damage free color removal process.
   Statement 17, kit. A kit of according to statements 1-16, kit, which further contains a hair damage repair additive from the class of organic acids, amino acids, quaternary of amine containing polymers, mineral or plant based oils, silicones. Optionally the amount of repair additive can be adjusted based on knowledge or analysis of the extent of damage of the hair to be treated.

### Systems

Statement 1, system. A system for creating a customized composition for coating a keratinous surface.
Statement 2, system. The system of statement 1, system to create a customized hair color composition for coloring hair from a series of compositions of statements 1-60E which function as standard compositions for combining to form the customized hair color composition.
Statement 3, system. The system of statement 2, system, wherein the customized composition is created from standard-compositions by analysis of the nature of the subject's hair prior to coloring, either through a person's assessment of the hair and or by a measurement device with said analysis of the nature of the subject's hair prior to coloring being used as input for determining the optimum amount of each of the standard compositions and/or the total amount of compositions for application to the hair.
Statement 4, system. The system of statement 2 and/or 3 wherein the customized composition created from the standard compositions is created automatically within a device and dispensed as a ready to use composition.
Statement 5, system. The system of any of statements 1-4, systems, which creates a plurality of hair colors by an automated color recipe prediction system which determines the mixtures of standard compositions, and which optionally also mixes and dispenses the one or more custom hair color composition.
Statement 6, system. A system for computer-assisted determination of a custom hair care composition according to any of statements 1-5, system, comprising:
a) a data processing apparatus;
b) providing at least one parameter of the hair prior to coloring to the data processing apparatus;
c) providing at least one parameter of the standard composition for coloring hair to the data processing apparatus;
d) optionally providing associated geolocation data to the data processing apparatus
e) determining at least one hair care composition which can maintain the hair color from data stored either directly or remotely to the data processing apparatus
f) presenting the recommended product or products to the user
g) optionally presenting the user with instructions on where and when to use the recommended products.
   Statement 7, system. The system of any of statements 1-6, system, for development of a dispenser of customized hair color compositions, further comprising analyzing head hair to determine the color and surface chemical and physical properties of the individual fibers and/or whole fiber assembly.
   Statement 8, system. A method of providing a customised hair color recipe using the output of any of the systems of statements 1-7, system, the method involving selection of appropriate levels and types of components including at least a binder, pigment, dispersant, and carrier according to the compositions of statements 1-65E and preparing/mixing these components at point of use, either manually or via an automated mixing device.
   Statement 9, system. A method of applying different recipes of customised hair color to different parts of the same head, comprising application of the systems and methods of statements 1-8 so as to enable different custom recipes for roots, midlengths and tips.
   Statement 10, system. The system of Statement 7 further comprising:
a user interface screen for receiving an input of a selected a hair color, wherein the screen displays a calculated color based on the plurality of ingredient levels;
a dispenser comprising:
a plurality of nozzles; and
a plurality of base color formulations;
a processor; and
at least one memory including instructions that, when executed by the one or more processors, cause the one or more processors to perform operations to:
receive, from the user interface screen, the input of ingredient levels for the displayed calculated hair color;
calculate a target amount for each base color formulation and additional formulation from the input of ingredient levels; and
transmit to the dispenser the calculated target amount for each base color formulation and additional formulation corresponding to a base color formulation and additional formulation.

1-47. (canceled)

48. A method for forming a color coating on keratin fibers comprising:
first, applying to the keratin fibers a pretreatment composition and at least partially drying to produce a first layer on the keratin fibers;
second, applying to the first layer sequentially, simultaneously or as a premix a first component and a second component to form a reactive colored composition overlaid on the first layer, and partially drying the reactive colored composition to produce a second layer with color on the keratin fibers;
third, applying to the second layer with color on the keratin fibers a topcoat and at least partially drying to form the third layer;
fourth, fully drying and curing the first, second and third layers to form the colored coating of first, second and third elastomeric layers; wherein,
a) the pretreatment composition comprises a first medium and an amine compound comprising an organic, silicone or organosilicone polymer having multiple amine groups in the polymer chain and/or as pendant groups, or a silane of one to twenty silicons having a distribution of organoamine groups and alkoxy silyl groups which upon drying and curing forms the first elastomeric layer;
b) the first component comprises a second medium and a silicone or organosilicone polymer having first reactive functional carboxyl groups;
c) the second component comprises the second medium and an organic in situ linking compound having second reactive functional carbodiimide groups;
d) one of the first and second components is combined with pigment particles and dispersant before applying in the second step;
e) the reactive colored composition formed from the combination of the first and second components forms upon curing in situ covalent linkages of at least acylurea groups and the second elastomer layer with color;
f) the first and second components each have a wt. avg molecular weight range of from about 120 Da to about 500 KDa and have first and second reactive functional group densities of at least about 1 per first and/or second component molecule;
g) the first and second components have in chain and/or pendant non-reactive functional groups that do not in situ react with the first and second reactive functional groups;
h) the topcoat comprises a third medium, topcoat material and one of a complementary pair of in situ reactive polymers, which pair comprises
i. the silicone or organosilicone polymer having first reactive functional carboxyl groups of the first component, and
ii. the organic in situ linking compound having second reactive functional carbodiimide groups of the second component,
iii. the ratio of the first reactive functional carboxyl groups to second reactive functional carbodiimide groups of the reactive colored composition is adjusted to account for the presence of h)i) or h)ii) in the topcoat;
i) the topcoat is converted upon drying and curing to form the third elastomer layer which is in situ covalently linked to the second elastomer layer with at least acylurea groups formed from h(i) or h(ii) with at least a portion of the corresponding complementary reactive functional groups of the first or second component of the reactive composition;
j) the pretreatment composition, the first component, the second component, the pigment particles and the topcoat are maintained in separate containers until they are applied according to first, second and third steps of applying.

49. The method according to claim 48 wherein prior to the first step for forming the color coating on keratin fibers;
first, cleansing the keratin fibers first with one or more of a cleaning agent, an ultrasound device or an electromagnetic frequency device, rinsing the keratin fibers to produce cleansed keratin fibers; and,
second, treating the cleansed keratin fibers with an atmospheric plasma generation device to produce plasma treated keratin fibers which become the keratin fibers of the first step.

50. The method according to claim 48 wherein the topcoat material comprises one or more of a UV filter, a visible light filter, a radical scavenger, a triplet formation inhibitor, a water repellant, a hair spray compound or polymeric microparticles having neutralized acid moieties enveloping pigment microparticles.

51. The method according to claim 48 wherein keratin fibers are human hair and the color coating on the keratin fibers has a remanence of at least 10 washing with shampoo, a rub off resistance of at least 30 strokes with a crockmeter, a resistance to sebum of at least 5 cycles, and a tactile and aural sensation about the same as that produced from untreated natural blond hair.

52. The method according to claim 51 wherein the keratin fibers with color coating comprise a coated swatch of human hair with one end of the swatch attached to a stanchion.

53. The method according to claim 52 wherein the coated swatch has a remanence of at least 10 washings with aqueous anionic detergent, a rub off resistance of at least 30 strokes with a crockmeter, a resistance to sebum of at least 5 cycles, and a tactile and aural sensation about the same as that produced from untreated natural blond hair.

54. The method according to claim 48 wherein the first and second reactive functional group densities range from about one per 1000 monomeric units to about one per 1 monomer unit.

55. The method according to claim 48 wherein the non-functional groups of the first and second components comprise urethane, urea, ester, amide, carbonate or any combination thereof.

56. The method according to claim 48 wherein the amine compound is polyethylene imine or aminoalkyl alkoxy silane of one to twenty silicons having a distribution of organoamine groups, alkoxy silyl groups, and alkyl groups.

57. The method according to claim 56 wherein the amine compound is an aminoalkyltrialkoxy silane wherein the alkyl group is 1 to 6 carbons, the alkoxy group is ethoxy or methoxy.

58. The method according to claim 48 wherein the pretreatment composition and/or the reactive colored composition further comprise a cellulose derivative that is soluble in ketone, alcohol or ester solvent and subsequent to the fourth step removing the pretreatment composition and/or the reactive colored coating by treating with a volatile ketone, alcohol or ester solvent.

59. The method according to claim 58 wherein both of the pretreatment composition and the reactive colored composition include a cellulose derivative comprising nitrocellulose and/or cellulose acetate-butyrate.

60. The method according to claim 48 further comprising removing the colored coating from the keratin fibers by treating the colored coating with a ultra UV electromagnetic radiation to cleave functional groups of the second elastomer layer that are susceptible to cleavage with UV radiation, or treating the colored coating with sodium fluoride to cleave functional groups of the second elastomer layer that are susceptible to cleavage with sodium fluoride, or treating the colored coating with aminoethanol in aqueous hydroxide to cleave functional groups of the second elastomer layer that are susceptible to cleavage with aminoethanol in aqueous hydroxide.

61. The method according to claim 48 wherein the pigment particles are a blended combination of organic and/or inorganic pigment microparticles of differing visible colors, shades and hues matched according to a color gamut for blends selected to include at least one or more of Pigment Yellow 83, Pigment Green 36, Pigment Blue 60, Pigment Blue 66, Pigment Blue 16, Pigment Black 6, Pigment White 6, Pigment Red 122, Pigment Red 5, Pigment Red 112, Pigment Violet 19, aluminum flakes, copper flakes, or brass flakes.

62. The method according to claim 51 wherein the remanence has a uniformity that is consistent across different regions of human hair.

63. The method according to claim 51 wherein the remanence has a non-uniformity that is inconsistent across different regions of human hair.

64. The method according to claim 48 wherein one or more of the elastomer layers have a surface energy less than 24 mN m⁻¹ and Hansen Solubility Parameters, δD, δP, δH that satisfy the equation: \sqrt{4*(δD−x)²+(δP−y)²+(δH−z)²}>4 wherein 16<x<17.2, 0.8<y<6.7 and 1.2<z<13.

65. A method according to claim 48 further comprising the steps of:
a) assessing the condition of the user's hair to provide first data,
b) assessing the users desired end hair color to provide second data,
c) comparing the first and second data to design and prepare a set of reactive colored compositions having differing pigments and/or pigment combinations to deliver the desired end color and performance on the user's hair.

66. A method according to claim 65 comprising selecting and applying one of the set of reactive colored compositions to a test section of keratin fibers having the first elastomer layer thereon to form a wet second color layer on the test section, at least partially drying the wet color layer, determining the suitability of the color relative to the second data, removing the at least partially dried wet color layer from the test section if the color is not suitable, or if suitable, applying the selected reactive colored composition to the remaining keratin fibers.
